(19) United States
(12) Patent Application Publication
(10) Pub. No.: US 2006/0216238 A1
(43) Pub. Date: Sep. 28, 2006

(54) Compositions and methods for targeting or imaging a tissue in a vertebrate subject

(76) Inventors: Marianne Manchester, San Diego, CA (US); Heidi Stuhlmann, San Diego, CA (US); John Lewis, Halifax (CA); Giuseppe Destito, San Diego, CA (US); John E. Johnson JR., San Diego, CA (US); Anju Chatterjee, San Diego, CA (US); Pratik Singh, San Diego, CA (US); Maria J. Gonzalez, San Diego, CA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 11/365,103

(22) Filed: Feb. 28, 2006

Related U.S. Application Data
(60) Provisional application No. 60/657,332, filed on Feb. 28, 2005.
(60) Provisional application No. 60/699,974, filed on Jul. 14, 2005.

(51) Int. Cl. A61K 49/10; A61K 48/00

(52) U.S. Cl. 424/9.34; 424/93.2; 977/802

(57) ABSTRACT

Compositions and methods are provided for targeting or imaging to a tumor or organ in a vertebrate subject. A plant viral particle for targeting and imaging and methods for treatment of disease are also provided.

### CROSS-REFERENCE TO RELATED APPLICATIONS

This application claims the benefit of U.S. Provisional Application No. 60/657,332, filed Feb. 28, 2005, and of U.S. Provisional Application No. 60/699,974, filed Jul. 14, 2005, the entire disclosure of which is incorporated herein by reference.

### STATEMENT OF GOVERNMENT SUPPORT

This invention was made by government support by The National Institutes of Health Grant Nos. AI47823 and N01-CO-17015-32. The Government has certain rights in this invention.

### FIELD

This invention generally relates to compositions and methods for targeting or imaging to a tumor or organ in a vertebrate subject. This invention further relates to a plant viral particle for targeting and imaging and to methods for treatment of disease.

### BACKGROUND

Fluorescent imaging in live animals has proven challenging due to the relatively poor tissue penetration of fluorescent signal. McDonald. and Choyke, Nat Med 9:713-25, 2003. In addition, time lapse studies in vivo require that probes have high fluorescent intensity with minimal toxicity or deleterious biological interactions. As a biological imaging sensor, plant viruses possess a number of distinct advantages over other particles. Cowpea mosaic virus is a small plant icosahedral virus that is composed of 60 identical copies of an asymmetric protein unit assembled around a bipartite single-stranded RNA genome. Goldbach. and Van Kammen, A. Molecular Plant Virology (ed. Davies.) 83-120, 1985.

Modern structural methods have revealed viruses to be fascinating chemical assemblies. Crick. and Watson, Nature 177:473-5, 1956. The atomic structure of CPMV has been determined to a 2.8 Å resolution (see FIG. 1), providing a defined structural environment suitable for chemical conjugation of dyes to the inside or outside surface of the viral particle. Lin et al., Virology 265:20-34, 1999. Each asymmetric protein unit possesses five accessible lysine residues that are positioned on the exterior of the assembled virus particle, and their reactivity with chemical reagents such as activated NHS esters provides a total of 300 addressable sites per virion (FIGS. 1b and 1c). Wang et al., Chem Biol 9:805-11, 2002; Chatterji et al., Chem Biol 11:855-63, 2004; U.S. Pat. No. 5,874,087; U.S. Pat. No. 5,958,422; U.S. Pat. No. 6,110,466.

A significant impediment to the widespread utilization of non-invasive in vivo vascular imaging techniques or the clinical application of molecular imaging is the poor sensitivity of current imaging probes. A need exists in the art to develop non-invasive in vivo vascular techniques as a diagnostic imaging platform and as a therapeutic platform that is tissue specific and sensitive enough to image at an increased depth into the organism and at an increased sensitivity.

### SUMMARY

This invention generally relates to methods of non-invasive in vivo targeting or imaging techniques of high sensitivity and the clinical application of molecular imaging with highly sensitive imaging probes. The non-invasive in vivo imaging techniques can be used for targeting or imaging tissue in a vertebrate subject, for example, for targeting or imaging a vasculature, organ or tumor of the subject. The invention further relates to methods for targeting or imaging to a tissue in a vertebrate subject comprising administering to the vertebrate subject a plant viral particle comprising a plurality of targeting/imaging molecules covalently attached to the viral particle, and delivering the targeting/imaging molecules on the viral particle to the tissue in the vertebrate subject.

A plant viral particle is provided comprising a viral subunit comprising a plurality of covalent attachment sites, a plurality of targeting/imaging molecules covalently attached to the viral subunit, and a plurality of viral subunits assembled into the viral particle displaying the plurality of targeting/imaging molecules on the viral particle. Methods for treating or preventing a disease in a vertebrate subject are provided comprising administering to the vertebrate subject a plant viral particle comprising a plurality of targeting elements directed to a cell surface receptor, wherein the targeting element binds to the cell surface receptor in a tumor or organ of the vertebrate subject.

A method for targeting or imaging a tissue in a vertebrate subject is provided comprising administering to the vertebrate subject a plant viral particle comprising a plurality of targeting/imaging molecules covalently attached to the viral particle, and delivering the targeting/imaging molecules on the viral particle to the tissue in the vertebrate subject. In a further aspect, the method comprises a viral subunit comprising a plurality of sites for the covalent attachment of the plurality of targeting/imaging molecules, and a plurality of viral subunits assembled into the viral particle displaying the plurality of targeting/imaging molecules on the viral particle. In a further aspect, the method provides that the plurality of targeting/imaging molecules are attached by chemical crosslink to the viral particle. In one aspect, a plurality of lysine residues on the viral subunit covalently attached to the plurality of targeting/imaging molecules. In a further aspect, N-hydroxysuccinimide ester covalently attaches the plurality of targeting/imaging molecules to the plurality of lysine residues on the viral subunit. In a further aspect, azide/alkyne cycloaddition in the presence of a metal ion and a ligand to the metal ion forms a triazole moiety thereby, and covalently attaches the plurality of targeting/imaging molecules to the plurality of lysine residues on the viral subunit. The tissue can be, for example, a tumor or organ in the vertebrate subject. The vertebrate subject can be, for example, a mammalian subject or an avian subject.

In a further aspect, the plurality of targeting/imaging molecules are displayed on the surface of the viral particle. In a further aspect, the plurality of targeting/imaging molecules are displayed on the interior of the viral particle.

In a detailed aspect, the plant viral particle is a Comovirus, Tombusvirus, Sobemovirus, or Nepovirus. In a further detailed aspect, the comovirus is a cowpea mosaic virus.

The method provides that the plurality of targeting/imaging molecules are small molecules, metal complexes, polymer, carbohydrates, polypeptides, polynucleotides, or fluorescent chemical molecule. In a detailed aspect, the plurality of targeting/imaging molecules are transferrin, RGD-containing polypeptide, protective antigen of anthrax toxin, neuropeptide Y, glycopolymer, polyethylene glycol, or folic acid. In a further aspect, the polypeptides are viral antigens or bacterial antigens. The polypeptides can be, for example, animal viral antigens or animal bacterial antigens.

The method provides that the plurality of targeting/imaging molecules are encoded by an exogenous nucleotide sequence in a viral particle genome. In a detailed aspect, the exogenous nucleotide sequence encodes siRNA, shRNA, or antisense RNA. In a further detailed aspect, the exogenous nucleotide sequence encodes a foreign polypeptide expressed as part of a coat protein of the viral particle. The exogenous nucleotide sequence encodes a foreign polypeptide expressed, for example, as part of a βE-αF loop, βB-βC loop, C′-C″ loop, or an N-terminus of the coat protein of the viral particle. In a detailed aspect, the foreign polypeptide is a tumor antigen, a viral antigen, a bacterial antigen, or a parasite antigen. In a further aspect, the plurality of targeting/imaging molecules are polypeptides binding a therapeutic or diagnostic agent. The plurality of targeting/imaging molecules can be, for example, peptides binding doxorubicin, verapamil, vincristine, or vinblastine.

The method further provides that the plurality of targeting/imaging molecules are ligands binding to tumor cell surface receptors. The plurality of targeting/imaging molecules can be, for example, ligands binding to VEGF-1 receptor, Flk-1/VEGF-2 receptor, LyP1 tumor target, targets to prostate endothelium or lung endothelium, α5β1 integrin, or αvβ3 integrin.

The method further provides that the plurality of targeting/imaging molecules induce a cell mediated immune response to a tumor cell, virus, bacteria, or parasite. The plurality of targeting/imaging molecules can be, for example, tumor antigens, viral antigens, bacterial antigens, or parasite antigens.

In a further embodiment, the method comprises detecting the targeting/imaging molecules on the viral particles in the vasculature. The targeting/imaging molecule can be, for example, a fluorescent molecule for fluorescent imaging, gadolinium chelate molecule for magnetic resonance imaging, PET contrast agent or CT contrast agent. The method further provides decreasing an immune response to the viral particles. The method provides coating the viral particles with polyethylene glycol or glucose. The viral particle can target or image blood flow in the vertebrate subject. The method further provides the viral particle that targets or images atherosclerosis, ischemia, or stroke in the mammal. In a further aspect, the plurality of targeting/imaging molecules are polypeptides. In a further aspect, the polypeptides are antibodies. The antibodies can target or image the viral particle, for example, to tumor specific antigens on a tumor in a live mammal. The antibodies can target or image the viral particle, for example, to VEGF-1 receptor, Flk-1/VEGF-2 receptor, LyP1 tumor target, lung endothelium, α5β1 integrin on colorectal carcinoma, nasopharyngeal carcinoma, αvβ3 integrin on breast, lung, brain, bone, liver, or kidney carcinomas.

The method further provides that the viral particle targets or images a vascular endothelium in the vertebrate subject. In a further aspect, the viral particle targets or images the vascular endothelium to distinguish veins from arteries. In a further aspect, the viral particle targets or images a tumor vasculature. In a further aspect, the viral particle targets or images embryonic vasculature. In a further detailed aspect, the plurality of targeting/imaging molecules are ligands binding to a receptor on the tumor vasculature. The plurality of targeting/imaging molecules can be, for example, ligands binding to VEGF-1 receptor or Flk-1/VEGF-2 receptor. In a detailed aspect, the viral particle inhibits angiogenesis in the tumor of the vertebrate subject.

The polypeptides target or image the viral particle, for example, to VEGF-1 receptor or Flk-1/VEGF-2 receptor on tumor vascular endothelium. The peptides target or image, for example, atherosclerosis, ischemia, or stroke.

The method further provides encapsidating a therapeutic or diagnostic agent in the viral particle. The therapeutic agent can be, for example, a nucleic acid, siRNA, shRNA, antisense RNA, dendrimer, aptamer, small molecule, polypeptide, or endotoxin. The therapeutic agent can treat, for example, vascular disease, atherosclerosis, ischemia, stroke, cancer or infectious disease. The therapeutic agent can be, for example, an anti-tumor agent, an anti-infective agent, an anti-angiogenesis agent, or an apoptosis inducer. The diagnostic agent can be, for example, a cell marker, green fluorescent protein, or luciferase.

A method for treating or preventing a disease in a vertebrate subject is provided comprising administering to the vertebrate subject a plant viral particle comprising a plurality of targeting/imaging molecules directed to a tissue of the vertebrate subject, wherein the targeting/imaging molecule binds to the tissue to treat or prevent the disease of the vertebrate subject.

In one aspect, the plurality of targeting/imaging molecules are ligands that binds to a cell surface receptor in the tissue of the vertebrate subject.

In a further aspect, the tissue is a vasculature in the vertebrate subject. The tissue can be a tumor vasculature in the vertebrate subject. In a detailed aspect, the cell surface receptor is VEGF-1 receptor or Flk-1/VEGF-2 receptor.

In a further aspect, the tissue can be a tumor in the vertebrate subject. In a detailed aspect, the cell surface receptor is VEGF-1 receptor, Flk-1/VEGF-2 receptor, LyP1 tumor target, lung endothelium, α5β1 integrin on colorectal carcinoma, nasopharyngeal carcinoma, αvβ3 integrin on breast, lung, brain, bone, liver, or kidney carcinomas. In a further detailed aspect, the plurality of targeting/imaging molecules are Egf17 polypeptides or fragments thereof.

The method further provides that the plurality of targeting/imaging molecules are exogenous polypeptides encoded by a viral particle genome. In a further aspect, the plurality of targeting/imaging molecules are polypeptides binding a therapeutic or diagnostic agent. In a detailed aspect, the plurality of targeting/imaging molecules are polypeptides binding doxorubicin, verapamil, vincristine, or vinblastine. The plurality of targeting/imaging molecules can be, for example, fluorescent dye, MRI contrast agent, PET contrast agent, or CT contrast agent. The method further provides that the plurality of targeting/imaging molecules are antibodies that binds to the cell surface receptor in the vasculature.

The method further provides that administering the plant viral particle to the subject via an oral, pulmonary, oropharyngeal, or nasopharyngeal route. The method further provides that administering the plant viral particle to the subject via parenteral, topical, intravenous, oral, subcutaneous, intraarterial, intracranial, intraperitoneal, intranasal or intramuscular route.

In a further aspect, the plurality of targeting/imaging molecules induce a cell mediated immune response to a tumor cell, virus, bacteria, or parasite. The plurality of targeting/imaging molecules can be, for example, tumor antigens, viral antigens, bacterial antigens, or parasite antigens.

In a further aspect, the disease is cancer, solid tumor or infectious disease. The method further provides that administering to the subject a therapeutic agent in the plant viral particle. The therapeutic agent can be, for example, a polypeptide, a nucleic acid, siRNA, shRNA, antisense RNA, dendrimer, aptamer, antibody, endotoxin, or a small molecule. The therapeutic agent can be, for example, an immune system modulator. The therapeutic agent can be, for example, an anti-tumor agent, an anti-infective agent, an anti-angiogenesis agent, or an apoptosis inducer. In a detailed aspect, the anti-tumor agent is doxorubicin, verapamil, vincristine, or vinblastine. The therapeutic agent can be, for example, IL-2, IL-3, IL-4, IL-5, IL-6, IL-7, IL-9, IL-12, IL-13, IL-15, interferon-α, interferon-β, interferon-γ, IP-10, I-TAC, MIG, functional derivatives of any thereof, or combinations of any two or more thereof. The therapeutic agent can be, for example, an enzyme, an interleukin, an interferon, a cytokine, a chemokine, TNF, taxol, an antibody, or combinations of any two or more thereof.

In a further aspect, the disease is a vascular disease. The vascular disease can be, for example, ischemia, stroke or atherosclerosis. The method further provides that administering to the subject a therapeutic agent in the plant viral particle. The therapeutic agent can be, for example, a polypeptide, a nucleic acid, siRNA, shRNA, antisense RNA, dendrimer, aptamer, antibody, endotoxin, or a small molecule.

A plant viral particle is provided comprising a viral subunit comprising a plurality of covalent attachment sites, a plurality of targeting/imaging molecules covalently attached to the viral subunit, and a plurality of viral subunits assembled into the plant viral particle displaying the plurality of targeting/imaging molecules on the plant viral particle. In a further aspect, the plant viral particle comprises a viral subunit comprising a plurality of sites for the covalent attachment of the plurality of targeting/imaging molecules, and a plurality of viral subunits assembled into the plant viral particle displaying the plurality of targeting/imaging molecules on the plant viral particle. In a further aspect, the plurality of targeting/imaging molecules are attached by chemical crosslink to the plant viral particle. In one aspect, a plurality of lysine residues on the viral subunit covalently attached to the plurality of targeting/imaging molecules. In a further aspect, N-hydroxysuccinimide ester covalently attaches the plurality of targeting/imaging molecules to the plurality of lysine residues on the viral subunit. In a further aspect, azide/alkyne cycloaddition in the presence of a metal ion and a ligand to the metal ion forms a triazole moiety thereby, and covalently attaches the plurality of targeting/imaging molecules to the plurality of lysine residues on the viral subunit. The tissue can be, for example, a tumor or organ in the vertebrate subject. The vertebrate subject can be, for example, a mammalian subject or an avian subject. The plant viral can target a tissue, for example, a tumor or organ in the vertebrate subject.

In a further aspect, the plurality of targeting/imaging molecules are displayed on the surface of the plant viral particle. In a further aspect, the plurality of targeting/imaging molecules are displayed on the interior of the plant viral particle.

In a detailed aspect, the plant viral particle is a Comovirus, Tombusvirus, Sobemovirus, or Nepovirus. In a further detailed aspect, the comovirus is a cowpea mosaic virus.

The method provides that the plurality of targeting/imaging molecules are small molecules, metal complexes, polymer, carbohydrates, polypeptides, polynucleotides, or fluorescent chemical molecule. In a detailed aspect, the plurality of targeting/imaging molecules are transferrin, RGD-containing polypeptide, protective antigen of anthrax toxin, neuropeptide Y, glycopolymer, polyethylene glycol, or folic acid. In a further aspect, the polypeptides are viral antigens or bacterial antigens. The polypeptides can be, for example, animal viral antigens or animal bacterial antigens.

The plant viral particle provides that the plurality of targeting/imaging molecules are encoded by an exogenous nucleotide sequence in a plant viral particle genome. In a detailed aspect, the exogenous nucleotide sequence encodes siRNA, shRNA, or antisense RNA. In a further detailed aspect, the exogenous nucleotide sequence encodes a foreign polypeptide expressed as part of a coat protein of the plant viral particle. The exogenous nucleotide sequence encodes a foreign polypeptide expressed, for example, as part of a βE-αF loop, βB-βC loop, C′-C″ loop, or an N-terminus of the coat protein of the plant viral particle. In a detailed aspect, the foreign polypeptide is a tumor antigen, a viral antigen, a bacterial antigen, or a parasite antigen. In a further aspect, the plurality of targeting/imaging molecules are polypeptides binding a therapeutic or diagnostic agent. The plurality of targeting/imaging molecules can be, for example, peptides binding doxorubicin, verapamil, vincristine, or vinblastine.

The plant viral particle further provides that the plurality of targeting/imaging molecules are ligands binding to tumor cell surface receptors. The plurality of targeting/imaging molecules can be, for example, ligands binding to VEGF-1 receptor, Flk-1/VEGF-2 receptor, LyP1 tumor target, targets to prostate endothelium or lung endothelium, α5β1 integrin, or αvβ3 integrin.

The plant viral particle further provides that the plurality of targeting/imaging molecules induce a cell mediated immune response to a tumor cell, virus, bacteria, or parasite. The plurality of targeting/imaging molecules can be, for example, tumor antigens, viral antigens, bacterial antigens, or parasite antigens.

In a further embodiment, the plant viral particle provides that the targeting/imaging molecules target or image a vasculature in a vertebrate subject. The targeting/imaging molecule can be, for example, a fluorescent molecule for fluorescent imaging, gadolinium chelate molecule for magnetic resonance imaging, PET contrast agent or CT contrast agent. The plant viral particle further has a decreased immune response. The plant viral particle can be coated, for example, with polyethylene glycol or glucose. The plant viral particle can target or image blood flow in the vertebrate subject. The plant viral particle is further provided that targets or images atherosclerosis, ischemia, or stroke in the mammal. In a further aspect, the plurality of targeting/imaging molecules are polypeptides. In a further aspect, the polypeptides are antibodies. The antibodies can target or image the plant viral particle, for example, to tumor specific antigens on a tumor in a live mammal. The antibodies can target or image the plant viral particle, for example, to VEGF-1 receptor, Flk-1/VEGF-2 receptor, LyP1 tumor target, lung endothelium, α5β1 integrin on colorectal carcinoma, nasopharyngeal carcinoma, αvβ3 integrin on breast, lung, brain, bone, liver, or kidney carcinomas.

The plant viral particle is further provided that targets or images a vascular endothelium in the vertebrate subject. In a further aspect, the plant viral particle targets or images the vascular endothelium to distinguish veins from arteries. In a further aspect, the plant viral particle targets or images a tumor vasculature. In a further aspect, the plant viral particle targets or images embryonic vasculature. In a further detailed aspect, the plurality of targeting/imaging molecules are ligands binding to a receptor on the tumor vasculature. The plurality of targeting/imaging molecules can be, for example, ligands binding to VEGF-1 receptor or Flk-1/VEGF-2 receptor. In a detailed aspect, the plant viral particle inhibits angiogenesis in the tumor of the vertebrate subject.

The polypeptides target or image the plant viral particle, for example, to VEGF-1 receptor or Flk-1/VEGF-2 receptor on tumor vascular endothelium. The polypeptides target or image, for example, atherosclerosis, ischemia, or stroke.

The plant viral particle are further provided that encapsidate a therapeutic or diagnostic agent in the plant viral particle. The therapeutic agent can be, for example, a nucleic acid, siRNA, shRNA, antisense RNA, dendrimer, aptamer, small molecule, polypeptide, or endotoxin. The therapeutic agent can treat, for example, vascular disease, atherosclerosis, ischemia, stroke, cancer or infectious disease. The therapeutic agent can be, for example, an anti-tumor agent, an anti-infective agent, an anti-angiogenesis agent, or an apoptosis inducer. The diagnostic agent can be, for example, a cell marker, green fluorescent protein, or luciferase.

### BRIEF DESCRIPTION OF THE DRAWINGS

The file of this patent contains at least one drawing/photograph executed in color. Copies of this patent with color drawing(s)/photograph(s) will be provided by the Office upon request and payment of the necessary fee.

FIGS. 1A, 1B, and 1C show the subunit organization of CPMV.

FIGS. 2A, 2B, 2C, 2D, and 2E show the reaction for attachment of dye to CPMV, ion exchange FPLC analysis and SDS-PAGE analysis of CPMV-AlexaFluor555 conjugate.

FIGS. 3A, 3B, 3C, and 3D show fluorescent dye-conjugated CPMV particles enable visualization of vasculature in living animals and fixed tissues.

FIGS. 4A, 4B, and 4C shows in vivo fluorescence imaging of chick CAM vasculature and evaluation of tumor angiogenesis in CAM/HT1080 fibrosarcoma model in live (a, b) and fixed (c) tissues.

FIGS. 5A and 5B show CPMV uptake is eliminated in chick embryos and reduced significantly in adult mice by PEG coating.

FIG. 6 shows particle stability in SGF and SIF.

FIG. 7 shows RT-PCR detection of CPMV RNA in mouse tissues.

FIGS. 8A, 8B, 8C, and 8D show characterization of Oregon Green-conjugated CPMV (OG-CPMV) particles.

FIG. 9 shows systemic trafficking in mice inoculated intravenously with OG-CPMV.

FIG. 10 shows systemic trafficking in mice inoculated orally with OG-CPMV.

FIG. 11 shows inactivation of CPMV infectivity by murine serum and plasma.

FIGS. 12A, 12B, 12C, 12D, 12E, and 12F show binding and uptake of CPMV particles by bone marrow dendritic cells.

FIGS. 13A, 13B, 13C, 13D, 13E, 13F, 13G, 13H, 13I, 13J, and 13K show binding and internalization of CPMV nanoparticles in vitro and in vivo.

FIG. 14 shows uptake of CPMV in vivo.

FIG. 15 shows a model of the CPMV structure showing the small coat protein pentamers in dark grey and the large coat pentamers in light grey.

FIGS. 16A, 16B, 16C, and 16D show protein analysis of the purified CPMV chimeric virions.

FIGS. 17A and 17B show cytokine expression following CPMV chimera immunization.

FIGS. 18A and 18B show CPMV chimera protection from a lethal virus challenge.

FIG. 19 shows a schematic representation of the RNA 2 of the CPMV genome to highlight the rationale for insertion of DOX binding peptide in the interior of the virus capsid.

FIG. 20A shows intrinsic tryptophan fluorescence of the wt and DOX-CPMV mutant.

FIG. 20B shows characterization of the DOX-CPMV mutant.

FIG. 20C shows fluorescence emission spectrum of the DOX loaded CPMV chimera.

FIG. 21 shows quantification of Doxorubicin molecules bound to DOX-CPMV chimera.

FIG. 22 shows cytotoxic effects of Doxorubicin exposure in HT29 cells as free drug or encapsulated in CPMV particles.

FIG. 23A shows confocal microscopic analysis of cells treated with the DOX-CPMV chimera.

FIG. 23B shows intracellular distribution of DOX bound CPMV particles.

FIG. 23C shows immuno-flourescence analysis of DOX treated MDA MB 231 cells.

FIG. 23D shows localization of CPMV to lysosomes.

FIG. 24 shows the release of DOX from CPMV particles as a function of pH.

FIGS. 25A and 25B show intactness of CPMV particles before and after the treatment with cells.

FIGS. 26A and 26B show interaction of CPMV-VEGFR1 chimera with a Fit-1 receptor antibody in ELISA.

FIGS. 27A and 27B show CPMV-VEGF chimera targeted to MDA-MB 231 cells.

FIG. 28 shows cell proliferation in in vitro angiogensis assays.

FIG. 29 shows cell migration in in vitro angiogensis assays.

FIGS. 30A, 30B, and 30C show immunofluorescence of CPMV-VEGFR1 chimera in mice.

FIG. 31 shows synthesis of glycopolymers and virus-polymer conjugates.

FIG. 32 shows a synthetic scheme for preparation of folate-PEG virus particles

FIG. 33 shows size exclusion FPLC analysis of CPMV-PEG-FA and CPMV-WT.

FIG. 34 shows TEM images of a purified preparation of folate-PEG CPMV virus showing intact particles.

FIG. 35 shows Western blots of wild type CPMV and CPMV-PEG-FA.

FIG. 36 shows fluorescence microscopy of HeLa cell monolayers were incubated with CPMV-PEG (A), CPMV-PEG-FA (B) or WT-CPMV (C).

FIG. 37 shows fluorescence microscopy of KB cell monolayers were incubated with CPMV-PEG (A), CPMV-PEG-FA (B) or WT-CPMV (C).

FIG. 38 shows measurement of virus binding to KB and HeLa cells using flow cytometry.

### DETAILED DESCRIPTION

This invention generally relates to methods of non-invasive in vivo imaging techniques of high sensitivity and the clinical application of molecular imaging with highly sensitive imaging probes. The non-invasive in vivo imaging techniques can be used for imaging organs or tumors in a vertebrate subject, for example, for imaging a vasculature of the subject. The invention further relates to methods for targeting or imaging to a tumor or organ in a vertebrate subject comprising administering to the vertebrate subject a plant viral particle comprising a plurality of targeting/imaging molecules covalently attached to the viral particle, and delivering the targeting/imaging molecules on the viral particle to the tumor or organ in the vertebrate subject. A plant viral particle is provided comprising a viral subunit comprising a plurality of covalent attachment sites, a plurality of targeting/imaging molecules covalently attached to the viral subunit, and a plurality of viral subunits assembled into the viral particle displaying the plurality of targeting/imaging molecules on the viral particle. Methods for treating or preventing a disease in a vertebrate subject are provided comprising administering to the vertebrate subject the plant viral particle comprising a plurality of targeting/imaging molecules covalently attached to the viral subunit.

A significant impediment to the widespread utilization of non-invasive in vivo vascular imaging techniques or the clinical application of molecular imaging is the poor sensitivity of current imaging probes. The present invention provides a viral nanoparticle composition and methods for vascular targeting or imaging using viral nanoparticles in a mammalian subject or an avian subject or methods for treating or preventing a disease in a mammalian subject or an avian subject using viral nanoparticles as a platform for the multivalent display of fluorescent dyes to image tissues deep inside the living organism. These bioavailable cowpea mosaic virus (CPMV)-based particles can be labeled to high densities, providing signal that is several orders of magnitude greater per particle than that of commercially available fluorescent dextrans or lectins. CPMV nanoparticles were used to visualize the vasculature of living mouse and chick embryos to a depth of several millimeters with an upright epifluorescence microscope. Visualization of human fibrosarcoma tumor angiogenesis in the chick embryo using fluorescent CPMV provided a means to identify arterial and venous vessels and to quantify the vascularization of the tumor microenvironment that is superior to other approaches. The multivalency of CPMV-based imaging sensors may be exploited to display a wide variety of tags, such as radioactive isotopes or MRI contrast agents.

The polyvalency of CPMV, combined with knowledge regarding the genetics, structure, bioavailability and reactivity of CPMV, makes this virus an ideal in vivo imaging sensor. Lin et al., Virology 265:20-34, 1999; Lomonossoff and Shanks, Embo J 2:2253-2258, 1983.; Porta et al., Virology 310:50-63, 2003.; Chatterji et al., Bioconjug Chem 15:807-13, 2004.; Brennan et al., Mol Biotechnol 17:15-26, 2001; Nicholas et al., Vaccine 20:2727-34, 2002, each incorporated herein by reference in its entirety.

The utility of in vivo CPMV-based imaging of the developing vasculature was assessed by injecting and visualizing mouse embryos and shell-free chick embryos. The potential of CPMV-based imaging was also evaluated in fixed tissues. In addition, fluorescent CPMV particles were introduced into adult mice by intraperitoneal or tail vein injection, and a survey of cryosections from various tissues was performed using an upright fluorescence microscope.

To further evaluate the efficacy of fluorescent CPMV particles, they were utilized to detect and visualize the extent of vascularization induced by HT1080 tumor on plants on the chick chorioallantoic membrane (CAM). These experiments demonstrated the greatly enhanced sensitivity provided by the high fluorescence output of the labeled CPMV particles compared to traditional vascular imaging agents such as fluorescent dextrans and lectins. Rizzo et al., Microvasc Res 46:320-32, 1993; Jilani et al., J. Histochem Cytochem 51:597-604, 2003, each incorporated herein by reference in its entirety.

It is to be understood that this invention is not limited to particular methods, reagents, compounds, compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural references unless the content clearly dictates otherwise. Thus, for example, reference to “a cell” includes a combination of two or more cells, and the like.

The term “about” as used herein when referring to a measurable value such as an amount, a temporal duration, and the like, is meant to encompass variations of ±20% or 110%, more preferably ±5%, even more preferably +1%, and still more preferably ±0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

“Vascular” refers to a system of blood vessels in a vertebrate subject, e.g., arteries, veins, and capillaries.

“Target”, “targeting,” or “target cell” refers to any cell, cell surface receptor, in a mammalian subject (e.g., a human) or an avian subject that can be bound to or targeted by a targeting/imaging molecule of the invention. The target cell can be, for example, a vascular endothelial cell, a tumor cell, or a receptor on a cell surface.

“Image” or “imaging” refers to a procedure that produces a picture of an area of the body, for example, organs, bones, tissues, or blood.

“Computed tomography (CT)” refers to a diagnostic imaging tool that computes multiple x-ray cross sections to produce a cross-sectional view of the vascular system, organs, bones, and tissues. “Positive emissions tomography (PET)” refers to a diagnostic imaging tool in which the patient receives a radioactive isotopes by injection or ingestion which then computes multiple x-ray cross sections to produce a cross-sectional view of the vascular system, organs, bones, and tissues to image the radioactive tracer. These radioactive isotopes are bound to compounds or drugs that are injected into the body and enable study of the physiology of normal and abnormal tissues.

“Magnetic resonance imaging (MRI)” refers to a diagnostic imaging tool using magnetic fields and radiowaves to produce a cross-sectional view of the body including the vascular system, organs, bones, and tissues.

“Plant viral particle” refers to any plant virus within the family of Comovirus which is a small plant icosahedral virus composed of 60 identical copies of an asymmetric protein subunit assembled around a bipartite single strand (+) RNA genome. Plant viral particles are plant viruses that include, but are not limited to, Comovirus, Tombusvirus, Sobemovirus, or Nepovirus. In one embodiment, the comovirus is cowpea mosaic virus.

“Patient”, “subject”, “vertebrate” or “mammal” are used interchangeably and refer to mammals such as human patients and non-human primates, as well as experimental animals such as rabbits, rats, and mice, and other animals. Animals include all vertebrates, e.g., mammals and non-mammals, such as sheep, dogs, cows, chickens, amphibians, and reptiles.

“Treating” or “treatment” includes the administration of the antibody compositions, compounds or agents of the present invention to prevent or delay the onset of the symptoms, complications, or biochemical indicia of a disease, alleviating the symptoms or arresting or inhibiting further development of the disease, condition, or disorder (e.g., cancer, or metastatic cancer). Treatment can be prophylactic (to prevent or delay the onset of the disease, or to prevent the manifestation of clinical or subclinical symptoms thereof) or therapeutic suppression or alleviation of symptoms after the manifestation of the disease.

In certain embodiments of the invention, the targeting/imaging moleucles of the invention, for example, can be coupled or conjugated to one or more therapeutic or cytotoxic moieties. “Cytotoxic moiety” refers to a moiety that inhibits cell growth or promotes cell death when proximate to or absorbed by a cell. Suitable cytotoxic moieties in this regard include radioactive agents or isotopes (radionuclides), chemotoxic agents such as differentiation inducers, inhibitors and small chemotoxic drugs, toxin proteins and derivatives thereof, as well as nucleotide sequences (or their antisense sequence). Therefore, the cytotoxic moiety can be, by way of non-limiting example, a chemotherapeutic agent, a photoactivated toxin or a radioactive agent.

In general, therapeutic agents can be conjugated to the targeting/imaging molecules of the invention, for example, by any suitable technique, with appropriate consideration of the need for pharmacokinetic stability and reduced overall toxicity to the patient. A therapeutic agent can be coupled to a suitable antibody moiety either directly or indirectly (e.g. via a linker group). A direct reaction between an agent and an antibody is possible when each possesses a functional group capable of reacting with the other. For example, a nucleophilic group, such as an amino or sulfhydryl group, can be capable of reacting with a carbonyl-containing group, such as an anhydride or an acid halide, or with an alkyl group containing a good leaving group (e.g., a halide). Alternatively, a suitable chemical linker group can be used. A linker group can function as a spacer to distance an antibody from an agent in order to avoid interference with binding capabilities. A linker group can also serve to increase the chemical reactivity of a substituent on a moiety or an antibody, and thus increase the coupling efficiency. An increase in chemical reactivity can also facilitate the use of moieties, or functional groups on moieties, which otherwise would not be possible.

“Delivering” refers to a property of the viral particles to target and image the vasculature of the vertebrate subject. More specifically, using the viral particles with the attached targeting/imaging molecules one can distinguish between arteries and veins within the vertebrate subject

“Covalent attachment” of the targeting/imaging molecule to the viral particle can occur through a variety of linkage chemistry to any of the lysine residues on the surface of the viral subunit. Each viral particle has 60 identical viral subunits. Each viral subunit has five available lysine residues for linkage to the targeting/imaging molecule.

Suitable linkage chemistries include maleimidyl linkers and alkyl halide linkers (which react with a sulfhydryl on the antibody moiety) and succinimidyl linkers (which react with a primary amine on the antibody moiety). Several primary amine and sulfhydryl groups are present on immunoglobulins, and additional groups can be designed into recombinant immunoglobulin molecules. It will be evident to those skilled in the art that a variety of bifunctional or polyfunctional reagents, both homo- and hetero-functional (such as those described in the catalog of the Pierce Chemical Co., Rockford, Ill.), can be employed as a linker group. Coupling can be effected, for example, through amino groups, carboxyl groups, sulfhydryl groups or oxidized carbohydrate residues (see, e.g., U.S. Pat. No. 4,671,958).

As an alternative coupling method, cytotoxic agents can be coupled to the targeting/imaging molecules of the invention, for example, through an oxidized carbohydrate group at a glycosylation site, as described in U.S. Pat. Nos. 5,057,313 and 5,156,840. Yet another alternative method of coupling the antibody and antibody compositions to the cytotoxic or imaging moiety is by the use of a non-covalent binding pair, such as streptavidin/biotin, or avidin/biotin. In these embodiments, one member of the pair is covalently coupled to the antibody moiety and the other member of the binding pair is covalently coupled to the cytotoxic or imaging moiety.

Where a cytotoxic moiety is more potent when free from the targeting/imaging molecules of the present invention, it can be desirable to use a linker group which is cleavable during or upon internalization into a cell, or which is gradually cleavable over time in the extracellular environment. A number of different cleavable linker groups have been described. The mechanisms for the intracellular release of a cytotoxic moiety agent from these linker groups include cleavage by reduction of a disulfide bond (e.g., U.S. Pat. No. 4,489,710); by irradiation of a photolabile bond (e.g., U.S. Pat. No. 4,625,014); by hydrolysis of derivatized amino acid side chains (e.g., U.S. Pat. No. 4,638,045); by serum complement-mediated hydrolysis (e.g., U.S. Pat. No. 4,671,958); and acid-catalyzed hydrolysis (e.g., U.S. Pat. No. 4,569,789).

It can be desirable to couple more than one therapeutic, cytotoxic and/or imaging moiety to a targeting/imaging molecule of the invention. By poly-derivatizing the CPMV plant viral particle of the invention, several cytotoxic strategies can be simultaneously implemented, an antibody can be made useful as a contrasting agent for several visualization techniques, or a therapeutic antibody can be labeled for tracking by a visualization technique. In one embodiment, multiple molecules of a cytotoxic moiety are coupled to one antibody molecule. In another embodiment, more than one type of moiety can be coupled to one antibody. For instance, a therapeutic moiety, such as an polynucleotide or antisense sequence, can be conjugated to an antibody in conjunction with a chemotoxic or radiotoxic moiety, to increase the effectiveness of the chemo- or radiotoxic therapy, as well as lowering the required dosage necessary to obtain the desired therapeutic effect. Regardless of the particular embodiment, immunoconjugates with more than one moiety can be prepared in a variety of ways. For example, more than one moiety can be coupled directly CPMV plant viral particle, that provide multiple sites for attachment (e.g., dendrimers) can be used. Alternatively, a carrier with the capacity to hold more than one cytotoxic moiety can be used.

As explained above, a carrier can bear the agents in a variety of ways, including covalent bonding either directly or via a linker group, and non-covalent associations. Suitable covalent-bond carriers include proteins such as albumins (e.g., U.S. Pat. No. 4,507,234), peptides, and polysaccharides such as aminodextran (e.g., U.S. Pat. No. 4,699,784), each of which have multiple sites for the attachment of moieties. A carrier can also bear an agent by non-covalent associations, such as non-covalent bonding or by encapsulation, such as within a liposome vesicle (e.g., U.S. Pat. Nos. 4,429,008 and 4,873,088). Encapsulation carriers are especially useful in chemotoxic therapeutic embodiments, as they can allow the therapeutic compositions to gradually release a chemotoxic moiety over time while concentrating it in the vicinity of the target cells.

Targeting Agents, Imaging Agents, and Therapeutic Agents

The methods for vascular targeting or imaging in a vertebrate subject and the methods for treating or preventing a disease in a vertebrate subject utilizing a CPMV plant viral particle with targeting, imaging, or therapeutic agents covalently attached on the surface of the CPMV viral particle. The targeting, imaging, or therapeutic agents include, but are not limited to, a nucleic acid, siRNA, shRNA, antisense RNA, dendrimer, aptamer, small molecule, polypeptide, or endotoxin. The targeting, imaging, or therapeutic agents further include, but are not limited to, anti-tumor agents, an anti-infective agents, an anti-angiogenesis agents, or apoptosis inducers.

Peptides possess appropriate pharmacokinetic properties to serve as cancer imaging or therapeutic targeting agents. Currently, only a small number of rationally-derived, labeled peptide analogues that target only a limited subset of antigens are available. Thus, finding new cancer targeting peptides is a central goal in the field of molecular targeting. Novel tumor-avid peptides can be efficiently identified via affinity selections using complex random peptide libraries containing millions of peptides that are displayed on bacteriophage. In vitro and in situ affinity selections can be used to identify peptides with high affinity for the target antigen in vitro. It has been found that peptides selected in vitro or in situ may not effectively target tumors in vivo due to poor peptide stability and other problems. To improve in vivo targeting, methodological combinatorial chemistry innovations allow selections to be conducted in the environment of the whole animal. Thus, new targeting peptides with optimal in vivo properties can be selected in vivo in tumor-bearing animals. In vivo selections have been proven successful in identifying peptides that target the vasculature of specific organs. In addition, in vivo selections have identified peptides that bind specifically to the surface of or are internalized into tumor cells. Direct selection of peptides for cancer imaging can be expedited using genetically engineered bacteriophage libraries that encode peptides with intrinsic radiometal-chelation or fluorescent sequences. J. Cell. Biochem. 90: 509-517, 2003.

The targeting, imaging, or therapeutic agents can be peptides or fragments thereof attached on the surface of CPMV viral particle for targeting of cell-surface receptors. Homing peptides that target or image the tumor vasculature, can be native peptides or peptides identified through phage display. For example, peptides having an NGR motif, or an AGG/HGG motif can target or image to prostrate epithelium or lung endothelium. J. Cell. Biochem. 90: 509-517, 2003.

The targeting, imaging, or therapeutic agents can be peptides or fragments thereof attached on the surface of CPMV viral particle for targeting of cell-surface receptors. Exemplary targeting peptides include peptide WHSDMEWWYLLG (F56) targeted to Flt-1/VEGFR-1 receptor. This targeting peptide has therapeutic activity to inhibit angiogenesis in solid tumor cells. Int J Cancer, 111: 165-73. 2004. Exemplary targeting peptides further include peptide ATWLPPR targeted to Flk-1/VEGFR-2 receptor. This targeting peptide has therapeutic activity to inhibit angiogenesis in solid tumor cells. EMBO J., 19:1525-1533, 2000. Exemplary targeting peptides further include peptide CGNKRTRGC (LyP1) having therapeutic activity as a tumor targeting peptide. Proc Natl Acad Sci USA., 101:9381-9386, 2004. Exemplary targeting peptides further include peptide CGFECVRQCPERC (GFE) having therapeutic activity targeting tumors in lung endothelium. Exemplary targeting peptides further include peptide CPIEDRPMC which binds α5β1 integrin on colorectal tumors (HT29). Exemplary targeting peptides further include peptide RLLDTNRPLLPY binds nasopharyngeal carcinoma cells. Exemplary targeting peptides further include cyclic RGD peptide which binds αvβ3 integrin on solid tumors including, but not limited to, breast, lung, brain, bone, liver, or kidney carcinomas. Exemplary targeting peptides or small molecules further include Egf17 protein or fragments thereof or small molecules based on Egf17 for targeting to cells involved in the early development of vascular endothelium. Dev Dyn., 230:316-324, 2004, each incorporated herein by reference in their entirety. Exemplary targeting peptides further include antibody Fab fragments or single chain Fv fragments that target tumor-specific antigens.

The targeting, imaging, or therapeutic agents can include molecular markers of angiogenesis. The polypeptides or fragments thereof are useful for targeting tumors and inhibiting angiogenesis in tumors of a vertebrate subject. and their targeting. Inhibitors of angiogenesis include, but are not limited to polypeptides or fragments thereof, for example, fibronectin, angiopoitein, arrestin, tumstatin. These polypeptides are endogenous inhibitors of angiogenesis which can be used as polypeptide fragments or agonists thereof.

The targeting, imaging, or therapeutic agents can be nucleic acids, small molecules, peptides or fragments thereof encapsidated within the CPMV viral particle for targeted delivery of the imaging and/or therapeutic agent. CPMV viral particle displaying peptides that target receptors would be taken up by target cells via receptor-mediated endocytosis. The therapeutic agents encapsidated within the CPMV viral particle can be a nucleic acid, including but not limited to siRNA, shRNA, antisense RNA that target and inactivate tumor or disease-specific genes. The therapeutic agents encapsidated within the CPMV viral particle can be one or more small molecules that activate apoptosis pathways. The therapeutic agents encapsidated within the CPMV viral particle can be one or more endotoxins, or other cytotoxic agents. The targeting, imaging, or therapeutic agents encapsidated within the CPMV viral particle can be cell markers that label the target cells in a stable fashion over a long period of time, for example, GFP marker or luciferase marker.

The targeting/imaging agents that can be incorporated into plant viral particles can be of highly diverse types and are subject only to the limitation that the nature and size of the foreign peptide and the site at which it is placed in or on the virus particle do not interfere with the capacity of the modified virus to assemble when cultured in vitro or in vivo. In broad concept, plant viral particles can comprise any biologically useful peptides (usually polypeptides) the function of which requires a particular conformation for its activity. In a further embodiment, this can be achieved by association of the peptide with a larger molecule, e.g., to improve its stability or mode of presentation in a particular biological system. Examples of such peptides include, but are not limited to, peptide hormones; enzymes; growth factors; antigens of protozoal, viral, bacterial, fungal or animal origin; antibodies including anti-idiotypic antibodies; immunoregulators and cytokines, eg interferons and interleukins; receptors; adhesions; and parts of precursors of any of the foregoing types of peptide. The peptide preferably contains more than 5 amino acids.

Among the broad range of bioactive peptide sequences presented on plant viral particles further include, but are not limited to, antigenic peptides which are the basis of vaccines, particularly animal (including human) virus vaccines. Vaccines can have prophylactic (i.e., disease prevention) or therapeutic (i.e., disease treatment) applications. For vaccine applications, an epitope presentation system provides that the antigenic peptide component will be sited appropriately on the virus particle so as to be easily recognized, by the immune system, for example by location on an exposed part of the coat protein of the virus. Plant viral particles containing an antigen derived from a pathogen, e.g., an animal virus, incorporated in an exposed position on the surface of the coat protein of the plant virus. This invention also comprises the use of such assembled modified plant virus particles as the immunogenic component of a vaccine. Such assembled plant viral particles presenting antigenic peptides also have applications as the antigen presentation component of an immunodiagnostic assay for detection of e.g., animal (including human) pathogens and diseases.

Certain viral infections and intracellular parasitic infections can be treated with plant viral particles containing an antigen derived from the pathogen. Chronic or acute infections such as those caused by adenovirus, cytomegalovirus, Epstein-Barr virus, herpes simplex virus 1, herpes simplex virus 2, human herpesvirus 6, varicella-zoster virus, hepatitis B virus, hepatitis D virus, papilloma virus, parvovirus B19, polyomavirus BK, polyomavirus JC, hepatitis C virus, measles virus, rubella virus, human immunodeficiency virus (HIV), human T cell leukemia virus I, and human T cell leukemia virus II persist in the host because the host is unable to mount a sufficient cytotoxic T-cell response against these viruses. Similarly, numerous parasites such as species of Leishmania, Toxoplasma, Trypanosoma, Plasmodium, Schistosoma, or Encephalitozoon persist in the host.

The targeting/imaging agents that can be incorporated into plant viral particles can be tumor-specific antigens including, but not limited to, any of the various MAGEs (Melanoma-Associated Antigen E), including MAGE 1 (e.g., GenBank Accession No. M77481), MAGE 2 (e.g., GenBank Accession No. U03735), MAGE 3, MAGE 4, etc.; any of the various tyrosinases; mutant ras; mutant p53 (e.g., GenBank Accession No. X54156 and AA494311); and p97 melanoma antigen (e.g., GenBank Accession No. M12154). Other tumor-specific antigens include the Ras peptide and p53 peptide associated with advanced cancers, the HPV 16/18 and E6/E7 antigens associated with cervical cancers, MUCI1-KLH antigen associated with breast carcinoma (e.g., GenBank Accession No. J03651), CEA (carcinoembryonic antigen) associated with colorectal cancer (e.g., GenBank Accession No. X98311), gp100 (e.g., GenBank Accession No. S73003) or MARTI antigens associated with melanoma, and the PSA antigen associated with prostate cancer (e.g., GenBank Accession No. X14810). The p53 gene sequence is known (See e.g., Harris et al. (1986) Mol. Cell. Biol., 6:4650-4656) and is deposited with GenBank under Accession No. M14694. Thus, the present invention can be used as immunotherapeutics for cancers including, but not limited to, cervical, breast, colorectal, prostate, lung cancers, and for melanomas.

The targeting/imaging agents that can be incorporated into plant viral particles can be viral antigens derived from known causative agents responsible for diseases including, but not limited to, measles, mumps, rubella, poliomyelitis, hepatitis A, B (e.g., GenBank Accession No. E02707), and C (e.g., GenBank Accession No. E06890), as well as other hepatitis viruses, influenza, adenovirus (e.g., types 4 and 7), rabies (e.g., GenBank Accession No. M34678), yellow fever, Japanese encephalitis (e.g., GenBank Accession No. E07883), dengue (e.g., GenBank Accession No. M24444), hantavirus, and AIDS (e.g., GenBank Accession No. U18552).

The targeting/imaging agents that can be incorporated into plant viral particles can be bacterial and parasitic antigens derived from known causative agents responsible for diseases including, but not limited to, diphtheria, pertussis (e.g., GenBank Accession No. M35274), tetanus (e.g., GenBank Accession No. M64353), tuberculosis, bacterial and fungal pneumonias (e.g., Haemophilus influenzae, Pneumocystis carinii, etc.), cholera, typhoid, plague, shigellosis, salmonellosis (e.g., GenBank Accession No. L03833), Legionnaire's Disease, Lyme disease (e.g., GenBank Accession No. U59487), malaria (e.g., GenBank Accession No. X53832), hookworm, onchocerciasis (e.g., GenBank Accession No. M27807), schistosomiasis (e.g., GenBank Accession No. L08198), trypanosomiasis, leshmaniasis, giardiasis (e.g., GenBank Accession No. M33641), amoebiasis, filariasis (e.g., GenBank Accession No. J03266), borreliosis, and trichinosis.

The targeting/imaging agents that can be incorporated into plant viral particles can be antibodies, monoclonal antibodies, polyclonal antibodies, single chain Fv antibodies, or fragments thereof, that target the plant viral particles to cells, tumor cells, viral pathogens, bacterial pathogens, or parasitic pathogens.

Labels or Detectable Groups as Targeting/Imaging Molecules

The particular label or detectable group used as a targeting/imaging molecule on the viral particle can be any fluorescent, radioactive isotopes, MRI contrast agents, enzymatic moieties, or detectable label of the invention. The detectable group can be any material having a detectable physical or chemical property. Such detectable labels have been well-developed in the field of fluorescent imaging, magnetic resonance imaging, positive emission tomography, or immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g. Dynabeads™), fluorescent dyes (e.g., fluorescein isothiocyanate, AlexaFluor555, Texas red, rhodamine, and the like), radiolabels (e.g., ³H, ¹⁴C, ³⁵S, ¹²⁵I, ¹²¹I, ¹¹²In, ⁹⁹mTc), other imaging agents such as microbubbles (for ultrasound imaging), ¹⁸F, ¹¹C, ¹⁵O, (for Positron emission tomography), ^{99m}TC, ¹¹¹In (for Single photon emission tomography), gadolinium chelate or iron (for magnetic resonance imaging), enzymes (e.g., horse radish peroxidase, alkaline phosphatase and others commonly used in an ELISA), and calorimetric labels such as colloidal gold or colored glass or plastic (e.g. polystyrene, polypropylene, latex, and the like) beads. Patents that described the use of such labels include U.S. Pat. Nos. 3,817,837; 3,850,752; 3,939,350; 3,996,345; 4,277,437; 4,275,149; and 4,366,241, each incorporated herein by reference in their entirety and for all purposes. See also Handbook of Fluorescent Probes and Research Chemicals, 6^{th} Ed., Molecular Probes, Inc., Eugene Oreg.

The label may be coupled directly or indirectly to the desired component of the assay according to methods well known in the art. As indicated above, a wide variety of labels may be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to an anti-ligand (e.g., streptavidin) molecule which is either inherently detectable or covalently bound to a signal system, such as a detectable enzyme, a fluorescent compound, or a chemiluminescent compound. A number of ligands and anti-ligands can be used. Where a ligand has a natural anti-ligand, for example, biotin, thyroxine, and cortisol, it can be used in conjunction with the labeled, naturally occurring anti-ligands. Alternatively, any haptenic or antigenic compound can be used in combination with an antibody.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidoreductases, particularly peroxidases. Fluorescent compounds include fluorescein and its derivatives, rhodamine and its derivatives, dansyl, umbelliferone, and the like Chemiluminescent compounds include luciferin, and 2,3-dihydrophthalazinediones, e.g., luminol. For a review of various labeling or signal producing systems which may be used, see, U.S. Pat. No. 4,391,904, incorporated herein by reference in its entirety and for all purposes.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple calorimetric labels may be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

Frequently, the targeting molecule will be labeled by joining, either covalently or non-covalently, to an imaging molecule that provides for a detectable signal.

Cytotoxic Agents

Preferred radionuclides for use as cytotoxic moieties are radionuclides which are suitable for pharmacological administration. Such radionuclides include ¹²³I, ¹²⁵I, ¹³¹I, ⁹⁰Y, ²¹¹At, ⁶⁷Cu, ¹⁸⁶Re, ¹⁸⁸Re, ²¹²Pb, and ²¹²Bi. Iodine and astatine isotopes are more preferred radionuclides for use in the therapeutic compositions of the present invention, as a large body of literature has been accumulated regarding their use. ¹³¹I is particularly preferred, as are other .beta.-radiation emitting nuclides, which have an effective range of several millimeters. ¹²³I, ¹²⁵I, ¹³¹I, or ²¹¹At can be conjugated to antibody moieties for use in the compositions and methods utilizing any of several known conjugation reagents, including Iodogen, N-succinimidyl 3-[²¹¹At]astatobenzoate, N-succinimidyl 3-[¹³¹I]iodobenzoate (SIB), and, N-succinimidyl 5-[¹³¹I]iodob-3-pyridinecarboxylate (SIPC). Any iodine isotope can be utilized in the recited iodo-reagents. Other radionuclides can be conjugated to the antibody or antibody compositions of the invention by suitable chelation agents known to those of skill in the nuclear medicine arts.

Preferred chemotoxic agents include small-molecule drugs such as methotrexate, and pyrimidine and purine analogs. Preferred chemotoxin differentiation inducers include phorbol esters and butyric acid. Chemotoxic moieties can be directly conjugated to the antibody or antibody compositions of the invention via a chemical linker, or can encapsulated in a carrier, which is in turn coupled to the antibody or antibody compositions of the invention.

Preferred toxin proteins for use as cytotoxic moieties include ricin, abrin, diphtheria toxin, cholera toxin, gelonin, Pseudomonas exotoxin, Shigella toxin, pokeweed antiviral protein, and other toxin proteins known in the medicinal biochemistry arts. As these toxin agents can elicit undesirable immune responses in the patient, especially if injected intravascularly, it is preferred that they be encapsulated in a carrier for coupling to the antibody and antibody compositions of the invention.

The cytotoxic moiety of the immunotoxin may be a cytotoxic drug or an enzymatically active toxin of bacterial or plant origin, or an enzymatically active fragment (“A chain”) of such a toxin. Enzymatically active toxins and fragments thereof used are diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolacca americana proteins (PAPI, PAPII, and PAP-S), momordica charantia inhibitor, curcin, crotin, sapaonaria officinalis inhibitor, gelonin, mitogellin, restrictocin, phenomycin, and enomycin. In another embodiment, the antibodies are conjugated to small molecule anticancer drugs. Conjugates of the monoclonal antibody and such cytotoxic moieties are made using a variety of bifunctional protein coupling agents. Examples of such reagents are SPDP, IT, bifunctional derivatives of imidoesters such a dimethyl adipimidate HCl, active esters such as disuccinimidyl suberate, aldehydes such as glutaraldehyde, bis-azido compounds such as bis(p-azidobenzoyl)hexanediamine, bis-diazonium derivatives such as bis-(p-diazoniumbenzoyl)-ethylenediamine, diisocyanates such as tolylene 2,6-diisocyanate, and bis-active fluorine compounds such as 1,5-difluoro-2,4-dinitrobenzene. The lysing portion of a toxin may be joined to the Fab fragment of antibodies.

Advantageously, the targeting/imaging molecules of the invention specifically binding the stathmin, can be conjugated to ricin A chain. Most advantageously the ricin A chain is deglycosylated and produced through recombinant means. An advantageous method of making the ricin immunotoxin is described in Vitetta et al., Science 238:1098, 1987, which is incorporated by reference in its entirety.

“Contacted” when applied to a cell is used herein to describe the process by which an antibody, antibody composition, cytotoxic agent or moiety, gene, protein and/or antisense sequence, is delivered to a target cell or is placed in direct proximity with the target cell. This delivery may be in vitro or in vivo and may involve the use of a recombinant vector system.

In another aspect, the present invention features an antibody or antibody composition of the invention, or a fragment thereof, conjugated to a therapeutic moiety, such as a cytotoxin, a drug (e.g., an immunosuppressant) or a radiotoxin. Such conjugates are referred to herein as “immunoconjugates”. Immunoconjugates which include one or more cytotoxins are referred to as “immunotoxins.” A cytotoxin or cytotoxic agent includes any agent that is detrimental to (e.g., kills) cells. Examples include anti-microtubule drugs of which the 2 main classes are taxols (paclitaxel, docetaxel) and vinca alkaloids (vincristine, vinblastine). Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin didne, mitoxantrone, mithramycin, actinomycin D, 1-dehydrotestosterone, glucocorticoids, procaine, tetracaine, lidocaine, propranolol, and puromycin and analogs or homologs thereof.

Suitable therapeutic agents for forming immunoconjugates of the invention include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). In a preferred embodiment, the therapeutic agent is a cytotoxic agent or a radiotoxic agent. In another embodiment, the therapeutic agent is an immunosuppressant. In yet another embodiment, the therapeutic agent is GM-CSF. In a preferred embodiment, the therapeutic agent is doxorubicin (adriamycin), cisplatin bleomycin sulfate, carmustine, chlorambucil, cyclophosphamide hydroxyurea or ricin A.

Antibodies

In some methods, the cell surface receptor and tumor antigen can be used to generate polyclonal and monoclonal antibodies, which are useful as described herein. A number of immunogens are used to produce antibodies that specifically bind cell surface receptor and tumor antigen polypeptides. Full-length cell surface receptor and tumor antigen polypeptides are suitable immunogens. Typically, the immunogen of interest is a peptide of at least about 3 amino acids, more typically the peptide is at least 5 amino acids in length, the fragment is at least 10 amino acids in length and typically the fragment is at least 15 amino acids in length. The peptides can be coupled to a carrier protein (e.g., as a fusion protein), or are recombinantly expressed in an immunization vector. Antigenic determinants on peptides to which antibodies bind are typically 3 to 10 amino acids in length. Naturally occurring polypeptides are also used either in pure or impure form. Recombinant polypeptides are expressed in eukaryotic or prokaryotic cells and purified using standard techniques. The polypeptide, or a synthetic version thereof, is then injected into an animal capable of producing antibodies. Either monoclonal or polyclonal antibodies can be generated for subsequent use in immunoassays to measure the presence and quantity of the polypeptide.

These antibodies find use in a number of applications. For example, the antibodies to cell surface receptor and tumor antigen can be coupled to standard affinity chromatography columns and used to purify cell surface receptor and tumor antigen proteins as further described below. The antibodies can also be used as blocking polypeptides, as outlined above, since they will specifically bind to the cell surface receptor and tumor antigen protein.

The anti-cell surface receptor and tumor antibodies can comprise polyclonal antibodies. Methods for producing polyclonal antibodies are known to those of skill in the art. In brief, an immunogen, for example, a purified polypeptide, a polypeptide coupled to an appropriate carrier (e.g., GST and keyhole limpet hemocyanin), or a polypeptide incorporated into an immunization vector such as a recombinant vaccinia virus (see, U.S. Pat. No. 4,722,848) is mixed with an adjuvant and animals are immunized with the mixture. The animal's immune response to the immunogen preparation is monitored by taking test bleeds and determining the titer of reactivity to the polypeptide of interest. When appropriately high titers of antibody to the immunogen are obtained, blood is collected from the animal and antisera are prepared. Further fractionation of the antisera to enrich for antibodies reactive to the polypeptide is performed where desired. See, e.g., Coligan, Current Protocols in Immunology, Wiley/Greene, NY, 1991; and Harlow and Lane, supra, each incorporated herein by reference in their entirety.

Antibodies, including binding fragments and single chain recombinant versions thereof, against predetermined fragments of cell surface receptor and tumor antigen proteins are raised by immunizing animals, e.g., with conjugates of the fragments with carrier proteins as described above.

The anti-cell surface receptor and tumor antibodies can, alternatively, be monoclonal antibodies. The monoclonal antibodies are prepared from cells secreting the desired antibody. These antibodies are screened for binding to normal or modified polypeptides, or screened for agonistic or antagonistic activity, e.g., activity mediated through the cell surface receptor and tumor antigen proteins. In some instances, it is desirable to prepare monoclonal antibodies from various mammalian hosts, such as mice, rodents, primates, and humans. Description of techniques for preparing such monoclonal antibodies are found in, e.g., Stites et al, eds., Basic and Clinical Immunology, 4^{th} ed., Lange Medical Publications, Los Altos, Calif., and references cited therein; Harlow and Lane, Supra; Goding, Monoclonal Antibodies: Principles and Practice, 2d ed., Academic Press, New York, N.Y., 1986; and Kohler et al., Nature 256:495-497, 1975, each incorporated herein by reference in its entirety. See Goding, Monoclonal Antibodies: Principles and Practice, Academic Press, pp. 59-103, 1986, incorporated herein by reference in its entirety.

Immortalized cell lines are those that fuse efficiently, support stable high level expression of antibody by the selected antibody-producing cells, and are sensitive to a medium such as HAT medium. More immortalized cell lines are murine myeloma lines, which can be obtained, for instance, from the Salk Institute Cell Distribution Center, San Diego, Calif. and the American Type Culture Collection, Rockville, Md. Human myeloma and mouse-human heteromyeloma cell lines also have been described for the production of human monoclonal antibodies (Kozbor, J. Immunol. 133:3001, 1984; Brodeur et al., Monoclonal Antibody Production Techniques and Applications, Marcel Dekker, Inc., New York, pp. 51-63, 1987, each incorporated herein by reference in its entirety).

The phrase “immune cell response” refers to the response of immune system cells to external or internal stimuli (e.g., antigen, cytokines, chemokines, and other cells) producing biochemical changes in the immune cells that result in immune cell migration, killing of target cells, phagocytosis, production of antibodies, other soluble effectors of the immune response, and the like.

“Endogenous” refers a protein, nucleic acid, lipid or other component produced within the body or within cells or organs of the body of a mammalian subject or an avian subject or originating within cells or organs of the body of a mammalian subject or an avian subject.

“Exogenous” refers a protein, nucleic acid, lipid, or other component originating outside the body of a mammalian subject or an avian subject.

“Immune response” refers to the concerted action of lymphocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells or tissues infected with pathogens, cancerous cells, or, in cases of pathological inflammation, or pain, hyperalgesia, allodynia, or nociceptive events in normal human cells or tissues.

“Signal transduction pathway” or “signal transduction event” refers to at least one biochemical reaction, but more commonly a series of biochemical reactions, which result from interaction of a cell with a stimulatory compound or agent. Thus, the interaction of a stimulatory compound with a cell generates a “signal” that is transmitted through the signal transduction pathway, ultimately resulting in a cellular response, e.g., an anti-nociceptive response described above.

Other suitable techniques involve selection of libraries of recombinant antibodies in phage or similar vectors. See, Huse et al., Science 246:1275-1281, 1989; and Ward et al., Nature 341:544-546, 1989, each incorporated herein by reference in its entirety. Also, recombinant immunoglobulins can be produced. See, U.S. Pat. No. 4,816,567; and Queen et al., Proc. Nat'l Acad. Sci. USA 86:10029-10033, 1989, each incorporated herein by reference in its entirety. See Winnacker, From Genes to Clones, VCH Publishers, N.Y., 1987, incorporated herein by reference in its entirety.

The vectors containing the polynucleotide sequences of interest (e.g., the heavy and light chain encoding sequences and expression control sequences) can be transferred into the host cell. Calcium chloride transfection is commonly utilized for prokaryotic cells, whereas calcium phosphate treatment or electroporation can be used for other cellular hosts. See generally Sambrook et al., Molecular Cloning: A Laboratory Manual, Cold Spring Harbor Press, 2d ed., 1989, incorporated herein by reference in its entirety. When heavy and light chains are cloned on separate expression vectors, the vectors are co-transfected to obtain expression and assembly of intact immunoglobulins. After introduction of recombinant DNA, cell lines expressing immunoglobulin products are cell selected. Cell lines capable of stable expression are useful (i.e., undiminished levels of expression after fifty passages of the cell line). See generally Scopes, Protein Purification, Springer-Verlag, N.Y., 1982, incorporated herein by reference in its entirety. Substantially pure immunoglobulins are of at least about 90 to 95% homogeneity, and are typically 98 to 99% homogeneity or more.

Frequently, the polypeptides and antibodies will be labeled by joining, either covalently or non-covalently, a substance which provides for a detectable signal. A wide variety of labels and conjugation techniques are known and are reported extensively in both the scientific and patent literature. Thus, an antibody used for detecting an analyte can be directly labeled with a detectable moiety, or can be indirectly labeled by, for example, binding to the antibody a secondary antibody that is, itself directly or indirectly labeled.

Antibodies are also used for affinity chromatography in isolating cell surface receptor and tumor antigen proteins. Columns are prepared, e.g., with the antibodies linked to a solid support, e.g., particles, such as agarose, Sephadex, or the like, where a cell lysate is passed through the column, washed, and treated with increasing concentrations of a mild denaturant, whereby purified cell surface receptor and tumor antigen polypeptides are released.

The protocol described by Huse is rendered more efficient in combination with phage-display technology. See, e.g., Dower et al., WO 91/17271; McCafferty et al., WO 92/01047; and U.S. Pat. Nos. 5,871,907; 5,858,657; 5,837,242; 5,733,743; and 5,565,332, each incorporated herein by reference in its entirety. In these methods, libraries of phage are produced in which members (display packages) display different antibodies on their outer surfaces. Antibodies are usually displayed as Fv or Fab fragments. Phage displaying antibodies with a desired specificity can be selected by affinity enrichment to the antigen or fragment thereof. Phage display combined with immunized transgenic non-human animals expressing human immunoglobulin genes can be used to obtain antigen specific antibodies even when the immune response to the antigen is weak.

In a variation of the phage-display method, human antibodies having the binding specificity of a selected murine antibody can be produced. See, for example, WO 92/20791, incorporated herein by reference in its entirety.

In another embodiment, fragments of antibodies against cell surface receptor and tumor antigen protein or protein analogs are provided. Typically, these fragments exhibit specific binding to the cell surface receptor and tumor antigen protein receptor similar to that of a complete immunoglobulin. Antibody fragments include separate heavy chains, light chains Fab, F_{ab′} F_{(ab′)2} and Fᵥ. Fragments are produced by recombinant DNA techniques, or by enzymic or chemical separation of intact immunoglobulins.

The antibodies can be monovalent antibodies. Methods for preparing monovalent antibodies are well known in the art. For example, one method involves recombinant expression of immunoglobulin light chain and modified heavy chain. The heavy chain is truncated generally at any point in the F_{c} region so as to prevent heavy chain crosslinking. Alternatively, the relevant cysteine residues are substituted with another amino acid residue or are deleted so as to prevent crosslinking.

An alternative approach is the generation of humanized immunoglobulins by linking the CDR regions of non-human antibodies to human constant regions by recombinant DNA techniques. See U.S. Pat. No. 5,585,089, incorporated herein by reference in its entirety. Humanized forms of non-human (e.g., murine) antibodies are immunoglobulins, immunoglobulin chains or fragments thereof (such as Fᵥ, F_{ab}, F_{ab′}, F_{ab2} or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fᵥ framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an F_{c} region, typically that of a human immunoglobulin. See Jones et al., Nature 321:522-525, 1986; Riechmann et al., Nature 332:323-329, 1988; and Presta, Curr. Op. Struct. Biol., 2:593-596, 1992, each incorporated herein by reference in its entirety.

Chimeric and humanized antibodies have the same or similar binding specificity and affinity as a mouse or other nonhuman antibody that provides the starting material for construction of a chimeric or humanized antibody. Chimeric antibodies are antibodies whose light and heavy chain genes have been constructed, typically by genetic engineering, from immunoglobulin gene segments belonging to different species. For example, the variable (V) segments of the genes from a mouse monoclonal antibody can be joined to human constant (C) segments, such as IgG₁ and IgG₄. Human isotype IgG₁ is typically used. A typical chimeric antibody is thus a hybrid protein consisting of the V or antigen-binding domain from a mouse antibody and the C or effector domain from a human antibody.

Humanized antibodies have variable region framework residues substantially from a human antibody (termed an acceptor antibody) and complementarity determining regions substantially from a mouse-antibody (referred to as the donor immunoglobulin). See, Queen et al., Proc. Natl. Acad. Sci. U.S.A. 86:10029-10033, 1989; and WO 90/07861; U.S. Pat. No. 5,693,762; U.S. Pat. No. 5,693,761; U.S. Pat. No. 5,585,089; U.S. Pat. No. 5,530,101; and U.S. Pat. No. 5,225,539, each incorporated herein by reference in its entirety. The constant region(s), if present, are also substantially or entirely from a human immunoglobulin. The human variable domains are usually chosen from human antibodies whose framework sequences exhibit a high degree of sequence identity with the murine variable region domains from which the CDRs were derived. The heavy and light chain variable region framework residues can be derived from the same or different human antibody sequences. The human antibody sequences can be the sequences of naturally occurring human antibodies or can be consensus sequences of several human antibodies. See WO 92/22653, incorporated herein by reference in its entirety. Certain amino acids from the human variable region framework residues are selected for substitution based on their possible influence on CDR conformation and/or binding to antigen. Investigation of such possible influences is by modeling, examination of the characteristics of the amino acids at particular locations, or empirical observation of the effects of substitution or mutagenesis of particular amino acids.

Bispecific antibodies are monoclonal, typically human or humanized, antibodies that have binding specificities for at least two different antigens. In the present case, one of the binding specificities is for the cell surface receptor and tumor antigen protein, the other one is for any other antigen, and for a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature 305:537-539, 1983). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J. 10:3655-3659, 1991. Each citation is incorporated herein by reference in its entirety.

The anti-cell surface receptor and tumor antibodies have various utilities. For example, anti-cell surface receptor and tumor antibodies can be used in diagnostic assays for a cell surface receptor and tumor antigen protein, e.g., detecting its expression in specific cells, tissues, or serum. Various diagnostic assay techniques can be used, such as competitive binding assays, direct or indirect sandwich assays and immunoprecipitation assays conducted in either heterogeneous or homogeneous phases (Zola, Monoclonal Antibodies: A Manual of Techniques, CRC Press, Inc., 1987, pp. 147-158,). The antibodies used in the diagnostic assays can be labeled with a detectable moiety. The detectable moiety should be capable of producing, either directly or indirectly, a detectable signal. For example, the detectable moiety can be a radioisotope, such as 3H, ¹⁴C, ³²P, ³⁵S, or ¹²⁵I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the detectable moiety can be employed, including those methods described by Hunter et al., Nature 144:945, 1962; David et al., Biochemistry 13:1014, 1974; Pain et al., J. Immunol. Meth. 40:219, 1981; and Nygren, J. Histochem. and Cytochem. 30:407, 1982. Each citation is incorporated herein by reference in its entirety.

A library of scFv antibodies to cell surface receptor and tumor antigen protein can be used to define the characteristics that would allow one to prospectively identify tumor cells and developing vasculature. One approach for a phage display library to identify an antibody composition that specifically binds to a cell surface receptor and tumor antigen protein, has been the use of scFv phage-libraries (see, e.g., Huston et al., Proc. Natl. Acad. Sci U.S.A., 85:5879-5883, 1988; Chaudhary et al., Proc. Natl. Acad. Sci U.S.A., 87:1066-1070, 1990. Various embodiments of scFv libraries displayed on bacteriophage coat proteins have been described. Refinements of phage display approaches are also known, for example, as described in WO96/06213 and WO92/01047 (Medical Research Council et al.) and WO97/08320 (Morphosys), which are incorporated herein by reference. The display of F_{ab} libraries is also known, for instance, as described in WO92/01047 (CAT/MRC) and WO91/17271 (Affymax).

Specific binding between an antibody or other binding agent and an antigen means a binding affinity of at least 10⁻⁶ M. Preferred binding agents bind with affinities of at least about 10⁻⁷ M, and preferably 10⁻⁸ M to 10⁻⁹ M, 10⁻¹⁰ M, 10⁻¹¹ M, or 10⁻¹² M. The term “epitope” means an antigenic determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

“Cancer” or “malignancy” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize) as well as any of a number of characteristic structural and/or molecular features. A “cancerous” or “malignant cell” is understood as a cell having specific structural properties, lacking differentiation and being capable of invasion and metastasis. Examples of cancers are, breast, lung, brain, bone, liver, kidney, colon, and prostate cancer. (see DeVita. et al., eds, Cancer Principles and Practice of Oncology, 6th. Ed., Lippincott Williams & Wilkins, Philadelphia, Pa., 2001; this reference is herein incorporated by reference in its entirety for all purposes).

“Advanced cancer” means cancer that is no longer localized to the primary tumor site, or a cancer that is Stage III or IV according to the American Joint Committee on Cancer (AJCC).

“Well tolerated” refers to the absence of adverse changes in health status that occur as a result of the treatment and would affect treatment decisions.

“Metastatic” refers to tumor cells, e.g., solid tumor cells, that are able to establish secondary tumor lesions in the brain, lungs, liver, or bone of immune deficient mice upon injection into the mammary fat pad and/or the circulation of the immune deficient mouse.

“Non-metastatic” refers to tumor cells, e.g., solid tumor cells, that are unable to establish secondary tumor lesions in the lungs, liver, bone or brain or other target organs of tumor metastasis in immune deficient mice upon injection into the mammary fat pad and/or the circulation. The human tumor cells used herein and addressed herein as non-metastatic are able to establish primary tumors upon injection into the mammary fat pad of the immune deficient mouse, but they are unable to disseminate from those primary tumors.

“Lymphocyte” as used herein has the normal meaning in the art, and refers to any of the mononuclear, nonphagocytic leukocytes, found in the blood, lymph, and lymphoid tissues, e.g., B and T lymphocytes.

“Epitope” refers to a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

An intact “antibody” comprises at least two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH₁, CH₂ and CH₃. Each light chain is comprised of a light chain variable region (abbreviated herein as LCVR or V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system. The term antibody includes antigen-binding portions of an intact antibody that retain capacity to bind stathmin. Examples of binding include (i) a Fab fragment, a monovalent fragment consisting of the V_{L}, V_{H}, C_{L} and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the V_{L} and V_{H} domains of a single arm of an antibody, (v) a dAb fragment (Ward et al., Nature 341:544-546, 1989), which consists of a VH domain; and (vi) an isolated complementarity determining region (CDR).

“Single chain antibodies” or “single chain Fv (scFv)” refers to an antibody fusion molecule of the two domains of the Fv fragment, V_{L} and V_{H}. Although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); See, e.g., Bird et al., Science 242:423-426, 1988; and Huston et al., Proc. Natl. Acad. Sci. USA, 85:5879-5883, 1988). Such single chain antibodies are included by reference to the term “antibody” fragments can be prepared by recombinant techniques or enzymatic or chemical cleavage of intact antibodies.

“Human sequence antibody” includes antibodies having variable and constant regions (if present) derived from human germline immunoglobulin sequences. The human sequence antibodies of the invention can include amino acid residues not encoded by human germline immunoglobulin sequences (e.g., mutations introduced by random or site-specific mutagenesis in vitro or by somatic mutation in vivo). Such antibodies can be generated in non-human transgenic animals, e.g., as described in PCT Publication Nos. WO 01/14424 and WO 00/37504. However, the term “human sequence antibody”, as used herein, is not intended to include antibodies in which CDR sequences derived from the germline of another mammalian species, such as a mouse, have been grafted onto human framework sequences (e.g., humanized antibodies).

Also, recombinant immunoglobulins may be produced. See, Cabilly, U.S. Pat. No. 4,816,567 incorporated herein by reference in its entirety and for all purposes; and Queen et al., Proc. Nat'l Acad. Sci. USA 86:10029-10033, 1989.

“Monoclonal antibody” refer to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions (if present) derived from human germline immunoglobulin sequences. In one embodiment, the human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

“Polyclonal antibody” refers to a preparation of more than 1 (two or more) different antibodies to a stathmin protein. Such a preparation includes antibodies binding to a range of different epitopes. Antibodies to stathmin can bind to an epitope on human stathmin so as to inhibit stathmin from interacting with a microtubule protein. These and other antibodies suitable for use in the present invention can be prepared according to methods that are well known in the art and/or are described in the references cited here. In preferred embodiments, anti-stathmin antibodies used in the invention are “human antibodies”—e.g., antibodies isolated from a human—or they are “human sequence antibodies” (defined supra).

“Immune cell response” refers to the response of immune system cells to external or internal stimuli (e.g., antigen, cell surface receptors, activated integrin receptors, cytokines, chemokines, and other cells) producing biochemical changes in the immune cells that result in immune cell migration, killing of target cells, phagocytosis, production of antibodies, other soluble effectors of the immune response, and the like.

“Immune response” refers to the concerted action of lymphocytes, antigen presenting cells, phagocytic cells, granulocytes, and soluble macromolecules produced by the above cells or the liver (including antibodies, cytokines, and complement) that results in selective damage to, destruction of, or elimination from the human body of cancerous cells, metastatic tumor cells, invading pathogens, cells or tissues infected with pathogens, or, in cases of autoimmunity or pathological inflammation, normal human cells or tissues.

“T lymphocyte response” and “T lymphocyte activity” are used here interchangeably to refer to the component of immune response dependent on T lymphocytes (e.g., the proliferation and/or differentiation of T lymphocytes into helper, cytotoxic killer, or suppressor T lymphocytes, the provision of signals by helper T lymphocytes to B lymphocytes that cause or prevent antibody production, the killing of specific target cells by cytotoxic T lymphocytes, and the release of soluble factors such as cytokines that modulate the function of other immune cells).

Components of an immune response can be detected in vitro by various methods that are well known to those of ordinary skill in the art. For example, (1) cytotoxic T lymphocytes can be incubated with radioactively labeled target cells and the lysis of these target cells detected by the release of radioactivity, (2) helper T lymphocytes can be incubated with antigens and antigen presenting cells and the synthesis and secretion of cytokines measured by standard methods (Windhagen et al., Immunity, 2:373-80, 1995), (3) antigen presenting cells can be incubated with whole protein antigen and the presentation of that antigen on MHC detected by either T lymphocyte activation assays or biophysical methods (Harding et al., Proc. Natl. Acad. Sci., 86:4230-4, 1989), (4) mast cells can be incubated with reagents that cross-link their Fc-epsilon receptors and histamine release measured by enzyme immunoassay (Siraganian et al., TIPS, 4:432-437, 1983).

Similarly, products of an immune response in either a model organism (e.g., mouse) or a human patient can also be detected by various methods that are well known to those of ordinary skill in the art. For example, (1) the production of antibodies in response to vaccination can be readily detected by standard methods currently used in clinical laboratories, e.g., an ELISA; (2) the migration of immune cells to sites of inflammation can be detected by scratching the surface of skin and placing a sterile container to capture the migrating cells over scratch site (Peters et al., Blood, 72:1310-5, 1988); (3) the proliferation of peripheral blood mononuclear cells in response to mitogens or mixed lymphocyte reaction can be measured using ³H-thymidine; (4) the phagocitic capacity of granulocytes, macrophages, and other phagocytes in PBMCs can be measured by placing PMBCs in wells together with labeled particles (Peters et al., Blood, 72:1310-5, 1988); and (5) the differentiation of immune system cells can be measured by labeling PBMCs with antibodies to CD molecules such as CD4 and CD8 and measuring the fraction of the PBMCs expressing these markers.

“Immunologically cross-reactive” or “immunologically reactive” refers to an antigen which is specifically reactive with an antibody which was generated using the same (“immunologically reactive”) or different (“immunologically cross-reactive”) antigen. Generally, the antigen is stathmin protein, or subsequence thereof.

“Immunologically reactive conditions” refers to conditions which allow an antibody, generated to a particular epitope of an antigen, to bind to that epitope to a detectably greater degree than the antibody binds to substantially all other epitopes, generally at least two times above background binding, preferably at least five times above background. Immunologically reactive conditions are dependent upon the format of the antibody binding reaction and typically are those utilized in immunoassay protocols. See, Harlow & Lane, Antibodies, A Laboratory Manual, Cold Spring Harbor Publications, New York, 1988 for a description of immunoassay formats and conditions.

Targets of interest for methods for treating or preventing a disease in a mammalian subject or an avian subject comprising administering to the mammalian subject or an avian subject a plant viral particle comprising a targeting element to metastatic cancer cells, e.g., solid tumor cells and metastasis, include, but are not limited to, microtubule binding proteins, growth factor receptors, antibodies, including anti-idiotypic antibodies and autoantibodies present in cancer, such as metastatic cancer. Other targets are adhesion proteins such as integrins, selecting, and immunoglobulin superfamily members. Springer, Nature, 346:425-433, 1990; Osborn, Cell, 62:3, 1990; Hynes, Cell, 69:11, 1992. Other targets of interest are growth factor receptors (e.g., FGFR, PDGFR, EGF, her/neu, NGFR, and VEGF) and their ligands. Other targets are G-protein receptors and include substance K receptor, the angiotensin receptor, the α- and β-adrenergic receptors, the serotonin receptors, and PAF receptor. See, e.g., Gilman, Ann. Rev. Biochem. 56: 625-649, 1987. Other targets include ion channels (e.g., calcium, sodium, potassium channels, channel proteins that mediate multidrug resistance), muscarinic receptors, acetylcholine receptors, GABA receptors, glutamate receptors, and dopamine receptors (see Harpold, U.S. Pat. No. 5,401,629 and U.S. Pat. No. 5,436,128). Other targets are cytokines, such as interleukins IL-1 through IL-13, tumor necrosis factors α- and β, interferons α-, β- and γ, tumor growth factor Beta (TGF-β), colony stimulating factor (CSF) and granulocyte monocyte colony stimulating factor (GM-CSF). See Aggrawal et al, eds., Human Cytokines: Handbook for Basic & Clinical Research, Blackwell Scientific, Boston, Mass., 1991. Other targets are hormones, enzymes, and intracellular and intercellular messengers, such as adenyl cyclase, guanyl cyclase, and phospholipase C. Drugs are also targets of interest. Target molecules can be human, mammalian or bacterial. Other targets are antigens, such as proteins, glycoproteins and carbohydrates from microbial pathogens, both viral and bacterial, and tumors. Still other targets are described in U.S. Pat. No. 4,366,241, incorporated herein by reference in its entirety and for all purposes. Some agents screened by the target merely bind to a target. Other agents agonize or antagonize the target.

RNA and DNA Interference Methods

Short Interfering RNAs (RNAi). RNA interference (RNAi) is a mechanism of post-transcriptional gene silencing mediated by double-stranded RNA (dsRNA), which is distinct from antisense and ribozyme-based approaches (see Jain, Pharmacogenomics 5:239-242 2004, for a review of RNAi and siRNA). RNA interference is useful in a method for treating a neoplastic disease or vascular disease state in a mammal by administering to the mammal a nucleic acid molecule (e.g., dsRNA) that hybridizes under stringent conditions to a neoplastic disease or vascular disease target gene, and attenuates expression of said target gene. dsRNA molecules are believed to direct sequence-specific degradation of mRNA in cells of various types after first undergoing processing by an RNase III-like enzyme called DICER (Bernstein et al., Nature 409:363, 2001) into smaller dsRNA molecules comprised of two 21 nt strands, each of which has a 5′ phosphate group and a 3′ hydroxyl, and includes a 19 nt region precisely complementary with the other strand, so that there is a 19 nt duplex region flanked by 2 nt-3′ overhangs. RNAi is thus mediated by short interfering RNAs (siRNA), which typically comprise a double-stranded region approximately 19 nucleotides in length with 1-2 nucleotide 3′ overhangs on each strand, resulting in a total length of between approximately 21 and 23 nucleotides. In mammalian cells, dsRNA longer than approximately 30 nucleotides typically induces nonspecific mRNA degradation via the interferon response. However, the presence of siRNA in mammalian cells, rather than inducing the interferon response, results in sequence-specific gene silencing.

In general, a short, interfering RNA (siRNA) comprises an RNA duplex that is preferably approximately 19 basepairs long and optionally further comprises one or two single-stranded overhangs or loops. An siRNA may comprise two RNA strands hybridized together, or may alternatively comprise a single RNA strand that includes a self-hybridizing portion. siRNAs may include one or more free strand ends, which may include phosphate and/or hydroxyl groups. siRNAs typically include a portion that hybridizes under stringent conditions with a target transcript. One strand of the siRNA (or, the self-hybridizing portion of the siRNA) is typically precisely complementary with a region of the target transcript, meaning that the siRNA hybridizes to the target transcript without a single mismatch. In certain embodiments of the invention in which perfect complementarity is not achieved, it is generally preferred that any mismatches be located at or near the siRNA termini.

siRNAs have been shown to downregulate gene expression when transferred into mammalian cells by such methods as transfection, electroporation, or microinjection, or when expressed in cells via any of a variety of plasmid-based approaches. RNA interference using siRNA is reviewed in, e.g., Tuschl, 2002, Nat. Biotechnol. 20:446-448; See also Yu et al, Proc. Natl. Acad. Sci., 99:6047-605, 2002; Sui et al., Proc. Natl. Acad. Sci USA., 99:5515-5520, 2002; Paddison et al, Genes and Dev. 16:948-958, 2002; Brummelkamp et al., 2002, Science 296, 550-553; Miyagashi and Taira, Nat. Biotech. 20:497-500, 2002; Paul et al., Nat. Biotech. 20:505-508, 2002. As described in these and other references, the siRNA may consist of two individual nucleic acid strands or of a single strand with a self-complementary region capable of forming a hairpin (stem-loop) structure. A number of variations in structure, length, number of mismatches, size of loop, identity of nucleotides in overhangs, etc., are consistent with effective siRNA-triggered gene silencing. While not wishing to be bound by any theory, it is thought that intracellular processing (e.g., by DICER) of a variety of different precursors results in production of siRNA capable of effectively mediating gene silencing. Generally it is preferred to target exons rather than introns, and it may also be preferable to select sequences complementary to regions within the 3′ portion of the target transcript. Generally it is preferred to select sequences that contain approximately equimolar ratio of the different nucleotides and to avoid stretches in which a single residue is repeated multiple times.

siRNAs may thus comprise RNA molecules having a double-stranded region approximately 19 nucleotides in length with 1-2 nucleotide 3′ overhangs on each strand, resulting in a total length of between approximately 21 and 23 nucleotides. As used herein, siRNAs also include various RNA structures that may be processed in vivo to generate such molecules. Such structures include RNA strands containing two complementary elements that hybridize to one another to form a stem, a loop, and optionally an overhang, preferably a 3′ overhang. Preferably, the stem is approximately 19 bp long, the loop is about 1-20, more preferably about 4-10, and most preferably about 6-8 nt long and/or the overhang is about 1-20, and more preferably about 2-15 nt long. In certain embodiments of the invention the stem is minimally 19 nucleotides in length and may be up to approximately 29 nucleotides in length. Loops of 4 nucleotides or greater are less likely subject to steric constraints than are shorter loops and therefore may be preferred. The overhang may include a 5′ phosphate and a 3′ hydroxyl. The overhang may but need not comprise a plurality of U residues, e.g., between 1 and 5 U residues. Classical siRNAs as described above trigger degradation of mRNAs to which they are targeted, thereby also reducing the rate of protein synthesis. In addition to siRNAs that act via the classical pathway, certain siRNAs that bind to the 3′ UTR of a template transcript may inhibit expression of a protein encoded by the template transcript by a mechanism related to but distinct from classic RNA interference, e.g., by reducing translation of the transcript rather than decreasing its stability. Such RNAs are referred to as microRNAs (mRNAs) and are typically between approximately 20 and 26 nucleotides in length, e.g., 22 nt in length. It is believed that they are derived from larger precursors known as small temporal RNAs (stRNAs) or mRNA precursors, which are typically approximately 70 nt long with an approximately 4-15 nt loop. (See Grishok et al, Cell 106:23-24, 2001; Hutvagner et al., Science 293:834-838, 2001; Ketting et al., Genes Dev. 15:2654-2659, 2001). Endogenous RNAs of this type have been identified in a number of organisms including mammals, suggesting that this mechanism of post-transcriptional gene silencing may be widespread (Lagos-Quintana et al., Science 294: 853-858, 2001; Pasquinelli, Trends in Genetics 18:171-173, 2002, and references in the foregoing two articles). MicroRNAs have been shown to block translation of target transcripts containing target sites in mammalian cells (Zeng et al., Molecular Cell 9: 1-20, 2002).

siRNAs such as naturally occurring or artificial (i.e., designed by humans) mRNAs that bind within the 3′ UTR (or elsewhere in a target transcript) and inhibit translation may tolerate a larger number of mismatches in the siRNA/template duplex, and particularly may tolerate mismatches within the central region of the duplex. In fact, there is evidence that some mismatches may be desirable or required as naturally occurring stRNAs frequently exhibit such mismatches as do mRNAs that have been shown to inhibit translation in vitro. For example, when hybridized with the target transcript such siRNAs frequently include two stretches of perfect complementarity separated by a region of mismatch. A variety of structures are possible. For example, the mRNA may include multiple areas of nonidentity (mismatch). The areas of nonidentity (mismatch) need not be symmetrical in the sense that both the target and the mRNA include nonpaired nucleotides. Typically the stretches of perfect complementarity are at least 5 nucleotides in length, e.g., 6, 7, or more nucleotides in length, while the regions of mismatch may be, for example, 1, 2, 3, or 4 nucleotides in length.

Hairpin structures designed to mimic siRNAs and mRNA precursors are processed intracellularly into molecules capable of reducing or inhibiting expression of target transcripts (McManus et al, RNA 8:842-850, 2002). These hairpin structures, which are based on classical siRNAs consisting of two RNA strands forming a 19 bp duplex structure are classified as class I or class II hairpins. Class I hairpins incorporate a loop at the 5′ or 3′ end of the antisense siRNA strand (i.e., the strand complementary to the target transcript whose inhibition is desired) but are otherwise identical to classical siRNAs. Class II hairpins resemble mRNA precursors in that they include a 19 nt duplex region and a loop at either the 3′ or 5′ end of the antisense strand of the duplex in addition to one or more nucleotide mismatches in the stem. These molecules are processed intracellularly into small RNA duplex structures capable of mediating silencing. They appear to exert their effects through degradation of the target mRNA rather than through translational repression as is thought to be the case for naturally occurring mRNAs and stRNAs.

Thus it is evident that a diverse set of RNA molecules containing duplex structures is able to mediate silencing through various mechanisms. For the purposes of the present invention, any such RNA, one portion of which binds to a target transcript and reduces its expression, whether by triggering degradation, by inhibiting translation, or by other means, is considered to be an siRNA, and any structure that generates such an siRNA (i.e., serves as a precursor to the RNA) is useful in the practice of the present invention.

In the context of the present invention, siRNAs are useful both for therapeutic purposes, e.g., to modulate the expression of a neoplastic disease or vascular disease protein in a subject at risk of or suffering from disease and for various of the inventive methods for the identification of compounds for treatment of a neoplastic disease or vascular disease that modulate the activity or level of the molecules described herein. In a preferred embodiment, the therapeutic treatment of tumor, atherosclerosis, ischemia, or stroke with an antibody, antisense vector, or double stranded RNA vector.

The invention therefore provides a method of inhibiting expression of a gene encoding a tumor, atherosclerosis, ischemia, or stroke related protein comprising the step of (i) providing a biological system in which expression of a gene encoding neoplastic disease or vascular disease protein is to be inhibited; and (ii) contacting the system with an siRNA targeted to a transcript encoding the protein. According to certain embodiments of the invention the protein is encoded by a gene within or linked to a neoplastic disease or vascular disease susceptibility locus, or within which a functional mutation causing or contributing to susceptibility or development of a neoplastic disease or vascular disease may exist. In other embodiments, neoplastic disease proteins or vascular disease proteins are inhibited. According to certain embodiments of the invention the biological system comprises a cell, and the contacting step comprises expressing the siRNA in the cell. According to certain embodiments of the invention the biological system comprises a subject, e.g., a mammalian subject such as a mouse or human, and the contacting step comprises administering the siRNA to the subject or comprises expressing the siRNA in the subject. According to certain embodiments of the invention the siRNA is expressed inducibly and/or in a cell-type or tissue specific manner.

By “biological system” is meant any vessel, well, or container in which biomolecules (e.g., nucleic acids, polypeptides, polysaccharides, lipids, and the like) are placed; a cell or population of cells; a tissue; an organ; an organism, and the like. Typically the biological system is a cell or population of cells, but the method can also be performed in a vessel using purified or recombinant proteins.

The invention provides siRNA molecules targeted to a transcript encoding any neoplastic disease or vascular disease protein. In particular, the invention provides siRNA molecules selectively or specifically targeted to a transcript encoding a polymorphic variant of such a transcript, wherein existence of the polymorphic variant in a subject is indicative of susceptibility to or presence of a neoplastic disease or vascular disease. The terms “selectively” or “specifically targeted to”, in this context, are intended to indicate that the siRNA causes greater reduction in expression of the variant than of other variants (i.e., variants whose existence in a subject is not indicative of susceptibility to or presence of a neoplastic disease or vascular disease. The siRNA, or collections of siRNAs, may be provided in the form of kits with additional components as appropriate.

Short hairpin RNAs (shRNA). RNA interference (RNAi), a mechanism of post-transcriptional gene silencing mediated by double-stranded RNA (dsRNA), is useful in a method for treating a neoplastic disease state in a mammal by administering to the mammal a nucleic acid molecule (e.g., dsRNA) that hybridizes under stringent conditions to a neoplastic disease or vascular disease target gene, and attenuates expression of said target gene. See Jain, K. K., 2004, Pharmacogenomics 5:239-42 for a review of RNAi and siRNA. A further method of RNA interference in the present invention is the use of short hairpin RNAs (shRNA). A plasmid containing a DNA sequence encoding for a particular desired siRNA sequence is delivered into a target cell via transfection or virally-mediated infection. Once in the cell, the DNA sequence is continuously transcribed into RNA molecules that loop back on themselves and form hairpin structures through intramolecular base pairing. These hairpin structures, once processed by the cell, are equivalent to transfected siRNA molecules and are used by the cell to mediate RNAi of the desired protein. The use of shRNA has an advantage over siRNA transfection as the former can lead to stable, long-term inhibition of protein expression. Inhibition of protein expression by transfected siRNAs is a transient phenomenon that does not occur for times periods longer than several days. In some cases, this may be preferable and desired. In cases where longer periods of protein inhibition are necessary, shRNA mediated inhibition is preferable.

Full and Partial Length Antisense RNA Transcripts. Antisense RNA transcripts have a base sequence complementary to part or all of any other RNA transcript in the same cell. Such transcripts have been shown to modulate gene expression through a variety of mechanisms including the modulation of RNA splicing, the modulation of RNA transport and the modulation of the translation of mRNA (Denhardt, N Y Acad. Sci. 660:70, 1992; Nellen, Trends Biochem. Sci. 18:419, 1993; Baker and Monia, Biochim. Biophys. Acta, 1489:3, 1993; Xu et al, Gene Therapy 7:438, 2000; French and Gerdes, Curr. Opin. Microbiol. 3:159, 2000; Terryn and Rouze, Trends Plant Sci. 5:1360, 2000)

Antisense RNA and DNA Oligonucleotides. Antisense nucleic acids are generally single-stranded nucleic acids (DNA, RNA, modified DNA, or modified RNA) complementary to a portion of a target nucleic acid (e.g., an mRNA transcript) and therefore able to bind to the target to form a duplex. Typically they are oligonucleotides that range from 15 to 35 nucleotides in length but may range from 10 up to approximately 50 nucleotides in length. Binding typically reduces or inhibits the function of the target nucleic acid. For example, antisense oligonucleotides may block transcription when bound to genomic DNA, inhibit translation when bound to mRNA, and/or lead to degradation of the nucleic acid. Reduction in expression of a neoplastic disease or vascular disease polypeptide may be achieved by the administration of antisense nucleic acids or peptide nucleic acids comprising sequences complementary to those of the mRNA that encodes the polypeptide. Antisense technology and its applications are well known in the art and are described in Phillips, ed., Antisense Technology, Methods Enzymol., Volumes 313 and 314, Academic Press, San Diego, 2000, and references mentioned therein. See also Crooke, ed., Antisense Drug Technology: Principles, Strategies, And Applications, 1^{st} Ed., Marcel Dekker; and references cited therein.

Antisense oligonucleotides can be synthesized with a base sequence that is complementary to a portion of any RNA transcript in the cell. Antisense oligonucleotides may modulate gene expression through a variety of mechanisms including the modulation of RNA splicing, the modulation of RNA transport and the modulation of the translation of mRNA (Denhardt, 1992). Various properties of antisense oligonucleotides including stability, toxicity, tissue distribution, and cellular uptake and binding affinity may be altered through chemical modifications including (i) replacement of the phosphodiester backbone (e.g., peptide nucleic acid, phosphorothioate oligonucleotides, and phosphoramidate oligonucleotides), (ii) modification of the sugar base (e.g., 2′-O-propylribose and 2′-methoxyethoxyribose), and (iii) modification of the nucleoside (e.g., C-5 propynyl U, C-5 thiazole U, and phenoxazine C) (Wagner, Nat. Medicine 1:1116, 1995; Varga et al., Immun. Lett. 69:217, 1999; Neilsen, Curr. Opin. Biotech. 10:71, 1999; Woolf, Nucleic Acids Res. 18:1763, 1990).

The invention provides a method of inhibiting expression of a gene encoding a neoplastic disease or vascular disease protein comprising the step of (i) providing a biological system in which expression of a gene encoding the protein is to be inhibited; and (ii) contacting the system with an antisense molecule that hybridizes to a transcript encoding neoplastic disease or vascular disease protein. According to certain embodiments of the invention the protein is encoded by a gene within or linked to a neoplastic disease or vascular disease susceptibility locus, or within which a functional mutation causing or contributing to a neoplastic disease or vascular disease or development of a neoplastic disease or vascular disease may exist. According to certain embodiments of the invention the biological system comprises a cell, and the contacting step comprises expressing the antisense molecule in the cell. According to certain embodiments of the invention the biological system comprises a subject, e.g., a mammalian subject such as a mouse or human, and the contacting step comprises administering the antisense molecule to the subject or comprises expressing the antisense molecule in the subject. The expression may be inducible and/or tissue or cell type-specific. The antisense molecule may be an oligonucleotide or a longer nucleic acid molecule. The invention provides such antisense molecules.

Ribozymes. Certain nucleic acid molecules referred to as ribozymes or deoxyribozymes have been shown to catalyze the sequence-specific cleavage of RNA molecules. The cleavage site is determined by complementary pairing of nucleotides in the RNA or DNA enzyme with nucleotides in the target RNA. Thus, RNA and DNA enzymes can be designed to cleave to any RNA molecule, thereby increasing its rate of degradation (Cotten and Birnstiel, EMBO J. 8:3861-3866, 1989; Usman et al., Nucl. Acids Mol. Biol. 10:243, 1996; Usman, et al, Curr. Opin. Struct. Biol. 1:527, 1996; Sun, et al., Pharmacol. Rev. 52:325, 2000. See also e.g., Cotten and Birnstiel, EMBO J. 8:3861-3866, 1989.)

The invention provides a method of inhibiting expression of a gene encoding a neoplastic disease or vascular disease protein comprising the step of (i) providing a biological system in which expression of a gene encoding the protein is to be inhibited; and (ii) contacting the system with a ribozyme that hybridizes to a transcript encoding the protein and directs cleavage of the transcript. According to certain embodiments of the invention the protein is encoded by a gene within or linked to a neoplastic disease or vascular disease susceptibility locus, or within which a functional mutation causing or contributing to susceptibility or development of neoplastic disease or vascular disease may exist. According to certain embodiments of the invention the biological system comprises a cell, and the contacting step comprises expressing the ribozyme in the cell. According to certain embodiments of the invention the biological system comprises a subject, e.g., a mammalian subject such as a mouse or human, and the contacting step comprises administering the ribozyme to the subject or comprises expressing the ribozyme in the subject. The expression may be inducible and/or tissue or cell-type specific according to certain embodiments of the invention. The invention provides ribozymes designed to cleave transcripts encoding neoplastic disease or vascular disease proteins, or polymorphic variants thereof, as described above.

Treatment Regimes

The invention provides method for vascular targeting or imaging in a mammalian subject or an avian subject administering to the mammal subject a plant viral particle comprising a plurality of targeting/imaging molecules covalently attached to the viral particle; and delivering the targeting/imaging molecules on the viral particles to the vasculature. The targeting/imaging molecules can be one or a combination of antibodies, e.g., antibodies to cell surface receptors, or tumor antigens (monoclonal, polyclonal or single chain Fv; intact or binding fragments thereof) or nucleic acid compositions, e.g., antisense oligonucleotides, double stranded RNA oligonucleotides (RNAi) or DNA oligonucleotides (vectors) containing nucleotide sequences encoding for the transcription of shRNA molecules, formulated together with a pharmaceutically acceptable carrier. Some compositions include a combination of multiple (e.g., two or more) monoclonal antibodies or antigen-binding portions thereof of the invention. In some compositions, each of the antibodies or antigen-binding portions thereof of the composition is a monoclonal antibody or a human sequence antibody that binds to a distinct, pre-selected epitope of an antigen.

In prophylactic applications, pharmaceutical compositions or medicaments are administered to a patient susceptible to, or otherwise at risk of a disease or condition (i.e., tumor, atherosclerosis, ischemia, or stroke) in an amount sufficient to eliminate or reduce the risk, lessen the severity, or delay the outset of the disease, including biochemical, histologic and/or behavioral symptoms of the disease, its complications and intermediate pathological phenotypes presenting during development of the disease. In therapeutic applications, compositions or medicants are administered to a patient suspected of, or already suffering from such a disease in an amount sufficient to cure, or at least partially arrest, the symptoms of the disease (biochemical, histologic and/or behavioral), including its complications and intermediate pathological phenotypes in development of the disease. An amount adequate to accomplish therapeutic or prophylactic treatment is defined as a therapeutically- or prophylactically-effective dose. In both prophylactic and therapeutic regimes, agents are usually administered in several dosages until a sufficient immune response has been achieved. Typically, the immune response is monitored and repeated dosages are given if the immune response starts to wane.

Effective Dosages

Effective doses of the antibody compositions of the present invention, e.g., antibodies to cell surface receptors, or tumor antigens (monoclonal, polyclonal or single chain Fv; intact or binding fragments thereof) or nucleic acid compositions, e.g., antisense oligonucleotides, double stranded RNA oligonucleotides (RNAi), or DNA oligonucleotides (vectors) containing nucleotide sequences encoding for the transcription of shRNA molecules, for the treatment of solid tumor tumor, atherosclerosis, ischemia, or stroke disease, described herein vary depending upon many different factors, including means of administration, target site, physiological state of the patient, whether the patient is human or an animal, other medications administered, and whether treatment is prophylactic or therapeutic. Usually, the patient is a human but nonhuman mammals including transgenic mammals can also be treated. Treatment dosages need to be titrated to optimize safety and efficacy.

For administration for vascular targeting or imaging in a mammalian subject or an avian subject utilizing a CPMV plant viral particle, the dosage ranges from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once per every two weeks or once a month or once every 3 to 6 months. In some methods, two or more CPMV plant viral particles with different binding specificities are administered simultaneously, in which case the dosage of each antibody administered falls within the ranges indicated. CPMV plant viral particle is usually administered on multiple occasions. Intervals between single dosages can be weekly, monthly or yearly. Intervals can also be irregular as indicated by measuring blood levels of antibody in the patient. In some methods, dosage is adjusted to achieve a plasma antibody concentration of 1-1000 μg/ml and in some methods 25-300 μg/ml. Alternatively, CPMV plant viral particles can be administered as a sustained release formulation, in which case less frequent administration is required. Dosage and frequency vary depending on the half-life of the antibody in the patient. In general, CPMV plant viral particle conjugated to human antibodies show the longest half life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

Doses for nucleic acids range from about 10 ng to 1 g, 100 ng to 100 mg, 1 μg to 10 mg, or 30-300 μg DNA per patient. Doses for CPMV plant viral particle vary from 10-100, or more, virions per dose.

Routes of Administration

Methods for vascular targeting or imaging in a mammalian subject or an avian subject utilizing CPMV plant viral particle conjugated to antibody compositions for inducing an immune response, e.g., antibodies to cell surface receptors, or tumor antigens, or nucleic acid compositions, e.g., antisense oligonucleotides, double stranded RNA oligonucleotides (RNAi), or DNA oligonucleotides (vectors) containing nucleotide sequences encoding for the transcription of shRNA molecules, for the treatment of tumor, atherosclerosis, ischemia, or stroke can be administered by parenteral, topical, intravenous, oral, subcutaneous, intraarterial, intracranial, intraperitoneal, intranasal or intramuscular means for prophylactic as inhalants for antibody preparations targeting tumor, atherosclerosis, ischemia, or stroke, and/or therapeutic treatment. The most typical route of administration of an immunogenic agent is subcutaneous although other routes can be equally effective. The next most common route is intramuscular injection. This type of injection is most typically performed in the arm or leg muscles. In some methods, CPMV plant viral particle can be administered intravenously or orally. In other methods, agents are injected directly into a particular tissue where a tumor is found, for example intracranial injection or convection enhanced delivery. Intramuscular injection or intravenous infusion are preferred for administration of CPMV plant viral particle. In some methods, antibodies conjugated to CPMV plant viral particle are administered as a sustained release composition or device, such as a Medipad™ device.

Agents of the invention can optionally be administered in combination with other agents that are at least partly effective in treating various diseases including various immune-related diseases. In the case of tumor, atherosclerosis, ischemia, or stroke, targeting or imaging molecules of the invention can also be administered in conjunction with other agents that increase passage of the agents of the invention into the vasculature.

Formulation

Methods for vascular targeting or imaging in a mammalian subject or an avian subject utilizing CPMV plant viral particle, e.g., antibodies to cell surface receptors, or tumor antigens (monoclonal, polyclonal or single chain Fv; intact or binding fragments thereof) or nucleic acid compositions, e.g., antisense oligonucleotides, double stranded RNA oligonucleotides (RNAi) or DNA oligonucleotides (vectors) containing nucleotide sequences encoding for the transcription of shRNA molecules, for the treatment of tumor, atherosclerosis, ischemia, or stroke, are often administered as pharmaceutical compositions comprising an active therapeutic agent, i.e., and a variety of other pharmaceutically acceptable components. See Remington's Pharmaceutical Science (15^{th} ed., Mack Publishing Company, Easton, Pa., 1980). The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

For parenteral administration, compositions of the invention can be administered as injectable dosages of a solution or suspension of the substance in a physiologically acceptable diluent with a pharmaceutical carrier that can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Antibodies can be administered in the form of a depot injection or implant preparation which can be formulated in such a manner as to permit a sustained release of the active ingredient. An exemplary composition comprises monoclonal antibody at 5 mg/mL, formulated in aqueous buffer consisting of 50 mM L-histidine, 150 mM NaCl, adjusted to pH 6.0 with HCl.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249:1527, 1990 and Hanes, Advanced Drug Delivery Reviews 28:97-119, 1997. The agents of this invention can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications.

For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

Topical application can result in transdermal or intradermal delivery. Topical administration can be facilitated by co-administration of the agent with cholera toxin or detoxified derivatives or subunits thereof or other similar bacterial toxins. Glenn et al., Nature 391: 851, 1998. Co-administration can be achieved by using the components as a mixture or as linked molecules obtained by chemical crosslinking or expression as a fusion protein.

Alternatively, transdermal delivery can be achieved using a skin patch or using transferosomes. Paul et al., Eur. J. Immunol. 25: 3521-24, 1995; Cevc et al., Biochem. Biophys. Acta 1368:201-15, 1998.

The pharmaceutical compositions are generally formulated as sterile, substantially isotonic and in full compliance with all Good Manufacturing Practice (GMP) regulations of the U.S. Food and Drug Administration.

Toxicity

Preferably, a therapeutically effective dose of the CPMV plant viral particle comprising a targeting/imaging molecule, e.g., antibodies to cell surface receptors, or tumor antigens (monoclonal, polyclonal or single chain Fv; intact or binding fragments thereof) or nucleic acid compositions, e.g., antisense oligonucleotides, double stranded RNA oligonucleotides (RNAi), or DNA oligonucleotides (vectors) containing nucleotide sequences encoding for the transcription of shRNA molecules, described herein will provide therapeutic benefit without causing substantial toxicity.

Toxicity of the proteins described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition. (See, e.g., Fingl et al, The Pharmacological Basis of Therapeutics, Ch. 1, 1975.)

Kits

Also within the scope of the invention are kits comprising the CPMV plant viral particle comprising a targeting/imaging molecule, e.g., antibodies to cell surface receptors, or tumor antigens (monoclonal, polyclonal or single chain Fv; intact or binding fragments thereof) or nucleic acid compositions, e.g., antisense oligonucleotides, double stranded RNA oligonucleotides (RNAi), or DNA oligonucleotides (vectors) containing nucleotide sequences encoding for the transcription of shRNA molecules) of the invention and instructions for use. The kit can further contain a least one additional reagent, or one or more additional human antibodies of the invention (e.g., a human antibody having a complementary activity which binds to an epitope in the antigen distinct from the first human antibody). Kits typically include a label indicating the intended use of the contents of the kit. The term label includes any writing, or recorded material supplied on or with the kit, or which otherwise accompanies the kit.

### EXEMPLARY EMBODIMENTS

### Example 1

Dye-Conjugated CPMV Particles

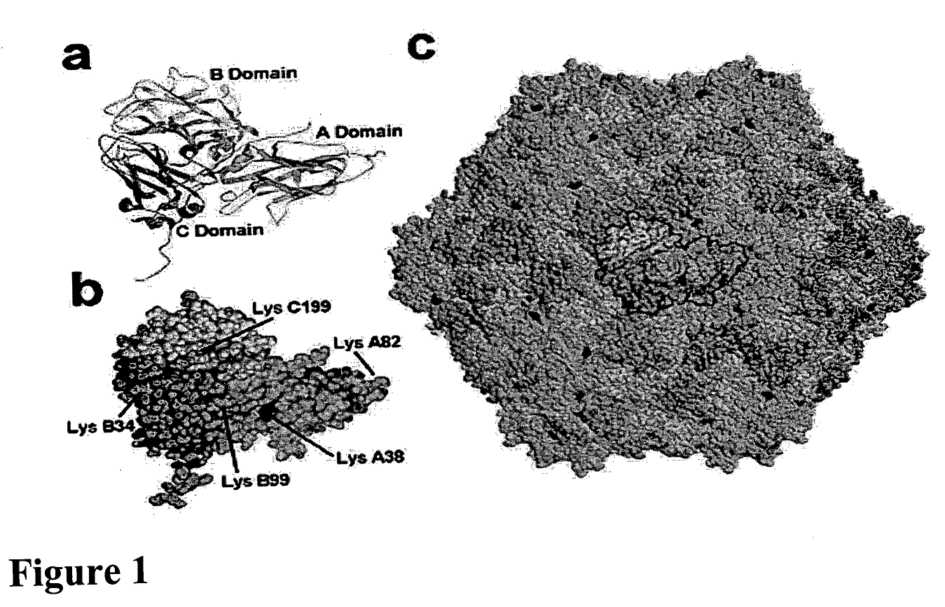

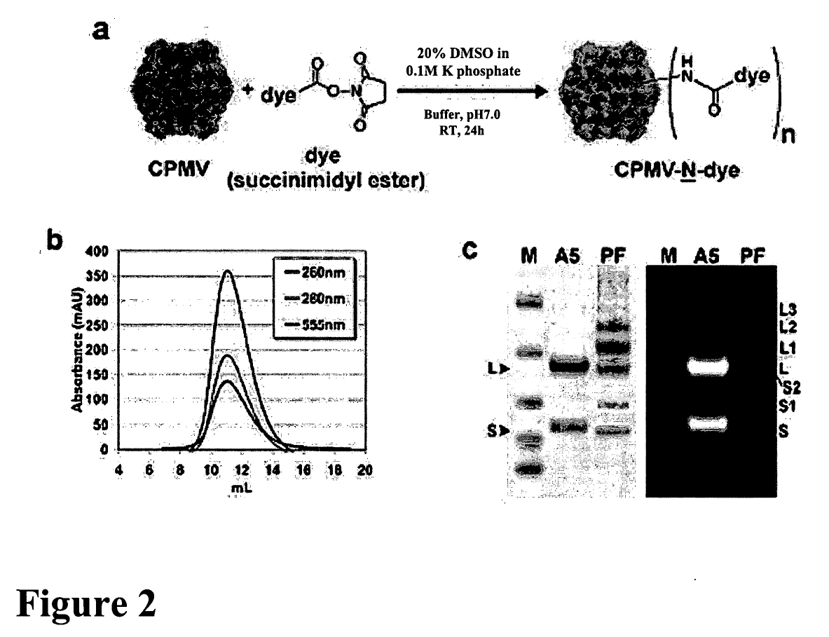

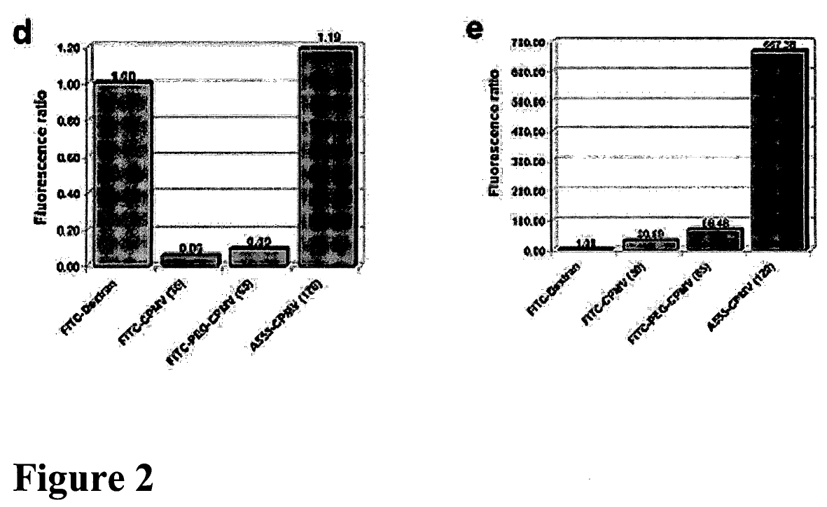

Wild-type CPMV particles were isolated and conjugated with the N-hydroxysuccinimide (NHS) ester of commercially available fluorescent dyes, taking advantage of the NHS ester's reactivity with surface lysines of CPMV (FIGS. 1, 2a). It has been previously described that lysine 38 of the small subunit (FIG. 1, red residues) is the most highly reactive using NHS chemistry, and attaining 100% occupancy at these sites is straightforward. Wang, Q. et al., Chem Biol 9:805-11, 2002. Conjugation at the remaining sites is enhanced by raising the pH and the ratio of dye to CPMV. Under highly forcing conditions (pH 8.3, 4000/1 dye/CPMV ratio), conjugation of up to 240 dyes per virus particle has been demonstrated. Wang et al., Chem Biol 9: 805-11, 2002, incorporated herein by reference in its entirety. Virus concentration is determined from the absorbance at 260 nm, and high purity of a virus preparation is indicated by a 260/280 ratio in the range of 1.6-1.8. Using a reaction stoichiometry of 50 dye molecules per virus asymmetric subunit for the AlexaFluor555 conjugation resulted in an average labeling of 120 dye molecules per virion (calculated from FIG. 2b, using an extinction coefficient of 8 for CPMV at 260 nm and 150000 for AlexaFluor555 at 555 nm). CPMV-AlexaFluor555 was analyzed on a size exclusion column and the 555 nm peak (AlexaFluor 555) eluted with the virus particles (FIG. 2b), which were detected at 260 and 280 nm. CPMV-FITC and CPMV-PEG-FITC particles were also prepared, and a measured labeling of 30 and 65 dyes/particle was achieved respectively. Examination of the fully denatured labeled virus by SDS-PAGE with both Coomassie staining and UV transillumination indicates that both the large and small subunits are conjugated to the fluorescent dye (FIG. 2c, lane A5). For CPMV-PEG-FITC, the small subunit is present both in an unmodified form and two shifted fluorescent species that represents conjugation to one or two PEG-FITC molecules (FIG. 2c, lane PF). A relatively small amount of the small subunit is conjugated to two PEG-FITC molecules. For the large subunit, the unmodified band and three fluorescent shifted bands are visualized. A small amount of the large subunit is conjugated to three PEG-FITC molecules, indicating that the principal products have one or two conjugated molecules of PEG-FITC (FIG. 2c, lane PF).

When the fluorescence output of the conjugated virus particles was measured in an in vitro fluorescence assay using the same microscope and imaging capture equipment used in the in vivo imaging experiments, it was determined that at an equal concentration the CPMV-AlexaFluor555 particles were 20% brighter than a commercially available 10 kDa FITC-dextran formulation (Molecular Probes) (FIG. 2d). While the FITC and PEG-FITC conjugated CPMV particles were significantly less bright than FITC-dextran at the same concentration, the measured fluorescence per molecule was roughly equivalent to the number of fluorophores per virus particle (FIG. 2e). This would indicate that no measurable fluorescence quenching is occurring. The high density labeling and excellent quantum efficiency of the AlexaFluor555 dye resulted in each CPMV-AlexaFluor555 particle being 667 times brighter than a corresponding particle of FITC-dextran (FIG. 2e).

FIG. 1a shows the subunit organization of CPMV; domain A (cyan) represents the small subunit while domains B (orange) and C (yellow) represent the two domains of the large subunit. b. Spacefilling model of subunit organization showing surface available lysine residues. Highly reactive lysine 38 of subunit A shown in red, less reactive lysines shown in pink. c. Spacefilling model of fully assembled CPMV particle showing subunit organization (block outlined) and surface available lysines. These images were created with the RasMol and DeepView programs using oligomer coordinates generated by the VIPER website derived from the X-ray crystal structure (ID code 1NY7). Sayle. and Milner-White, Trends Biochem Sci 20: 374, 1995; Schwede et al., Nucleic Acids Res 31:3381-5, 2003; Redd. et al., J Virol 75:11943-7, 2001, each incorporated herein by reference in its entirety.

FIG. 2a shows reaction for attachment of dye to CPMV. b. Ion exchange FPLC analysis of CPMV-AlexaFluor555 conjugate. Fluorescence emission at 555 nm elutes with the virus particles (260 and 280 nm). This indicates successful conjugation and enables the calculation of absolute concentrations. Using the extinction coefficients of the constituents (CPMV=8.0, AlexaFluor55=150000), the number of dyes per virus particle can be calculated. c. SDS-PAGE analysis of CPMV-AlexaFluor555 (A5) and CPMV-PEG-FITC (PF) conjugates. Mobility of unmodified large (42 kD) and small (24 kD) virus subunits are indicated by black arrowheads. The marker (M) is Biorad broad range prestained standard. Both panels are the same gel, the left with Coomassie Blue staining and the right panel (dark background) is under UV illumination to detect conjugated fluorescent dye. Unmodified subunits are visible with Coomassie but not under UV (S, L). In the A5 lanes, strong signal indicates efficient conjugation of AlexaFluor555 to both large and small subunits. In the PF lanes, multiple bands are present. Either one or two molecules of PEG-FITC (indicated by the fluorescent bands S1 and S2) conjugated to the small subunit (S), while one, two, or three molecules of PEG-FITC (indicated by L1, L2 and L3) conjugated to the large subunit (L). d. Fluorescence quantitation by concentration of dextrans and dye-labeled CPMV under the Zeiss Axioplan2 upright microscope. e. Fluorescence quantitation per molecule.

### Example 2

Detection of Fluorescent CPMV in Fixed Tissues

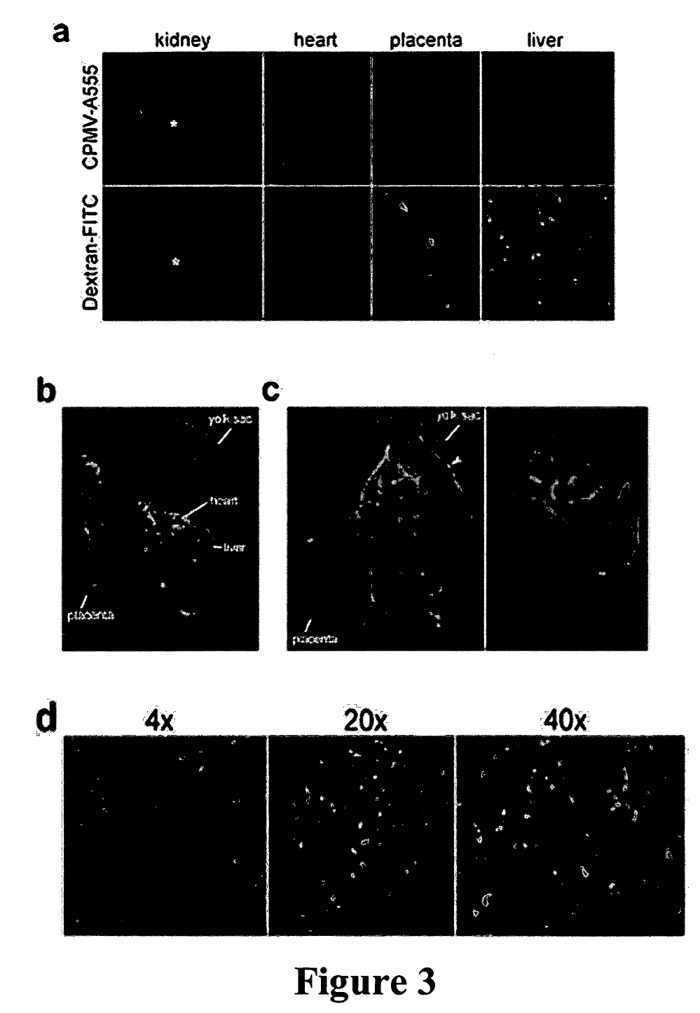

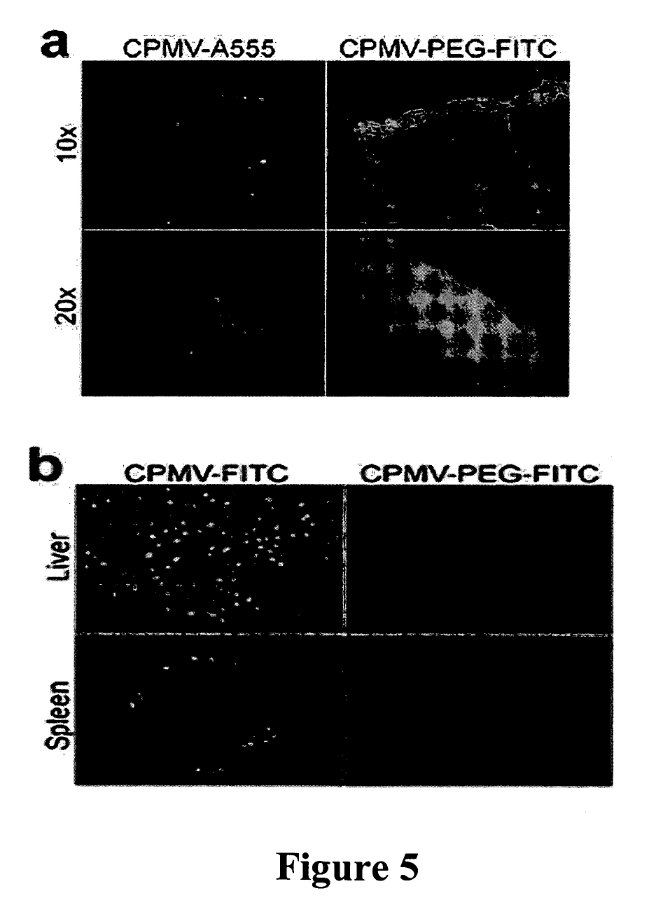

Whether the fluorescent CPMV particles could be visualized in the adult mouse vasculature after tail vein injection was examined. Of specific concern were the levels of dye-labeled CPMV required for adequate detection and whether the virus particles would persist in the circulation for extended periods of time. Injections of as little as 50 μg and up to 1 mg per animal were performed using either CPMV-AlexaFluor555 or a solution of 10 kDa FITC-dextran, and each was allowed to circulate from 5 minutes up to 72 hours before examining tissue distribution. Significant levels of fluorescence were detected in all tissue sections examined. The fluorescent CPMV seemed to associate preferentially with the lumen periphery of the vasculature (FIG. 3a, kidney), allowing excellent resolution of vascular structures in various organs including kidney, heart, placenta, and liver (FIG. 3a, top panels), whereas the 10 kDa FITC-dextran showed a more dispersed and uneven signal (FIG. 3a, bottom panels). No CPMV particles were detected outside of the vasculature up to 12 hours post-injection. When pregnant females were injected and embryos at E9.5 to E15.5 were examined, no fluorescent signal was found at any virus dosage or time point in the embryos or the fetal-derived areas of the placenta, suggesting that the CPMV particles cannot pass the placental barrier. It is of interest to note that both the CPMV-AlexaFluor555 and FITC-dextran began to accumulate in the liver (FIG. 3a and FIG. 5b) and spleen (FIG. 5b) almost immediately after tail vein injection, and were gradually depleted from the circulation over 72 h. No deleterious effects (thrombosis, malaise, death) were observed in the mice at any point during the experiments.

### Example 3

In Vivo Fluorescent Imaging of Developing Vasculature in Mouse Embryos

Since there was no appreciable transfer of CPMV particles from mother to embryo, it was necessary to deliver the CPMV particles directly into the embryonic circulation. Embryos between E9.5 and E15.5 (the morning of the vaginal plug is day E0.5) of development were surgically removed with the yolk sac and placenta intact. An injection technique was utilized whereby a glass microinjection needle was used to puncture and cannulate one of the small venules on the surface of the yolk sac. Modulation of the injection volume was controlled by a microadjustable syringe pump. This method resulted in minimal bleeding of the mouse embryo. When 10 μg of CPMV-AlexaFluor555 was injected, the particles circulated rapidly throughout the embryonic vasculature. Under the fluorescent microscope, resolution of the fine vascular structure of the yolk sac was possible from low magnification (1.5×, FIG. 3c) up to high resolution (40×, FIG. 3d) despite embryo movement. This was facilitated by the excellent signal-to-noise ratio of the dye-labeled CPMV particles. When the yolk sac was carefully removed to avoid rupturing any of the larger vessels, in vivo imaging of the living embryo vasculature was possible to a depth of a millimetre or more, particularly at earlier stages (E9.5-E10.5) of vascular development (FIG. 3c, right panel). It was also possible to monitor blood flow in real time at high frame rates due to the short required exposure time (<5 ms). It appeared that starting at the time of injection and increasing over time, a portion of the fluorescent signal was taken up by specific circulating blood cells, and to a lesser degree, cells at the blood vessel periphery. This effect was most noticeable in areas of low blood velocity.

Subsequent to in vivo imaging, the embryos were fixed and embedded for cryosectioning. The sections exhibited bright staining throughout the vasculature of the embryo and the fetal-derived portion of the placenta (FIG. 3b). The images obtained from these sections were of sufficient quality and detail to generate a 3D reconstruction of the embryonic vasculature in any region of interest using the appropriate software package (Volocity, Improvision).

FIG. 3 shows fluorescent dye-conjugated CPMV particles enable visualization of vasculature in living animals and fixed tissues. a. Fluorescence images of tissue cryosections from kidney (star indicates vessel lumen), heart, placenta and liver isolated from adult mice co-injected with CPMV-AlexaFluor555 and dextran-FITC. b. Cryosection of an 11.5d mouse embryo perfused with CPMV-AlexaFluor555. c. In vivo imaging of CPMV-AlexaFluor555 perfused 11.5d embryo with the yolk sac intact (left) and removed (right). White arrowhead indicates injection point. d. Fluorescent CPMV particles are restricted to the vasculature of the mouse embryo, and large vessels and capillaries are readily detectable. Favorable signal/noise ratio allows clear in vivo imaging at the full range of magnifications (4-40×) despite embryo movement.

### Example 4

### In Vivo Fluorescent Imaging of Chick Embryos

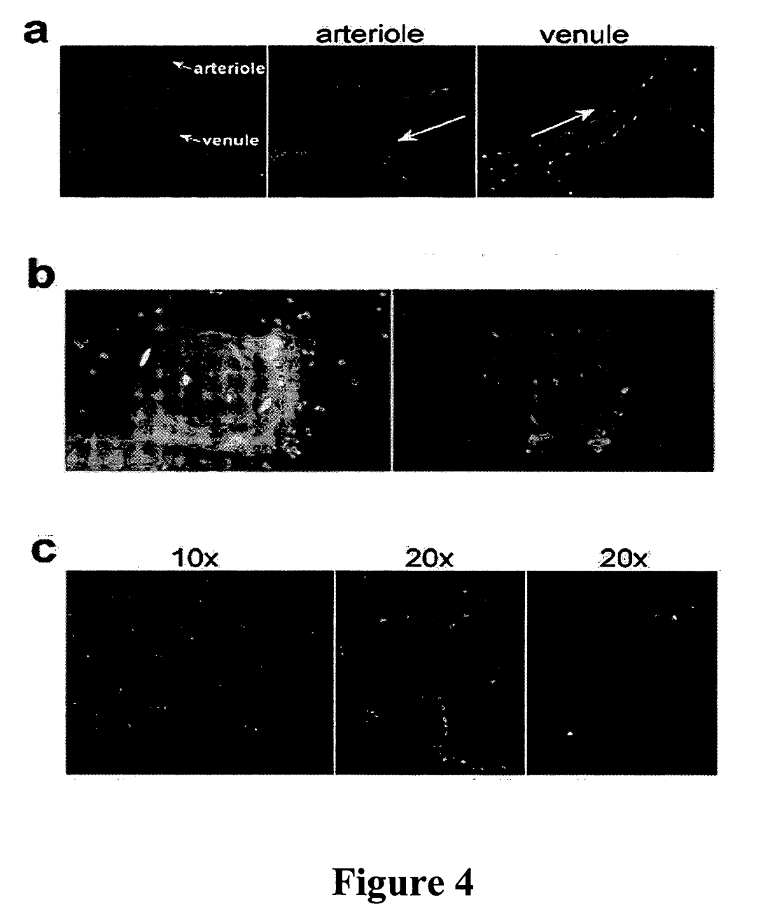

Shell-free chick embryos at 10 days of development were injected with 50 μg of CPMV-AlexaFluor555, and visualized with a Zeiss Axioplan2 upright microscope. The particles distributed throughout the embryo within 2 minutes, and provided sufficient fluorescence to visualize the vasculature throughout the CAM to a depth of approximately 0.5 cm (FIG. 4a, left). Cellular uptake of the fluorescent virus particles by peripheral cells of the vasculature and a subpopulation of circulating blood cells was more pronounced in the chick embryo than the mouse embryo. Of interest is the observation that while cellular uptake was not observed in the arterial vessels (FIG. 4a, middle), it was quite apparent in the venous vessels (FIG. 4a, right). This permitted quick identification of venous vasculature in both the live animals and fixed tissues.

### Example 5

HT1080 Tumor Angiogenesis

Human fibrosarcoma HT1080 cells embedded in a small collagen onplant placed into the CAM of a shell-free chick embryo induce vascularization of this collagen microenvironment (FIG. 4b). Seandel et al., Blood 97:2323-32, 2001, incorporated herein by reference in its entirety. Chick embryos bearing 7 day HT1080-containing onplants were injected with fluorescently labeled lectin, dextran, and CPMV. While the fluorescently labeled lectin outlines the vascular walls by binding to endothelial cells, it gives no indication of flow and provides a signal that is too weak to allow deep tissue visualization. Fluorescently labeled dextran and CPMV both circulated freely through the tumor vasculature. However, CPMV not only provides a much brighter signal, it can also be used to label the veins, thus giving a means to identify vascular origin and directionality within the tissue (FIG. 4b, right). In the 20 μm tumor sections the CPMV-AlexaFluor555 labeled vasculature is readily identified, thereby providing a means to visualize, identify, and quantify the vascularization of the tumor microenvironment (FIG. 4c).

FIG. 4 shows in vivo fluorescence imaging of chick CAM vasculature and evaluation of tumor angiogenesis in CAM/HT1080 fibrosarcoma model in live (a,b) and fixed (c) tissues. a. left, 10× image showing multiple levels of CAM, through capillary bed and larger vessels below to arterioles and venules (indicated). Center, 20× image shows blood flow in CAM arteriole and right, CAM venule (arrows denote blood flow direction). b. Left, bright field image of HT1080 tumor CAM onplant at 7 days. Opaque object is a nylon mesh grid used for quantifying angiogenesis. Right, fluorescence image of tumor onplant after injection of embryo with CPMV-AlexaFluor555. c. Cryosection of CAM/HT1080 tumor, nuclear stain (DAPI) in blue, CPMV-AlexaFluor555 in red. Extent of vascularization of onplant tumors can be identified and quantified. Lumen (indicated by star) and periphery of tumor blood vessels can be clearly delineated.

### Example 6

Coating CPMV with PEG Eliminates the Uptake of CPMV by the Reticuloendothelial System and Vascular Cells

Injected CPMV particles, regardless of the dye used for conjugation, were taken up by cells of the vasculature of both mouse and chick embryos, particularly in the chick. In addition, these particles accumulated in both the liver and spleen of the adult mouse (FIG. 5b, left panels). While uptake by the reticuloendothelial system is non-specific, it is not clear whether uptake by cells of the vasculature is the result of a specific interaction or a non-specific factor such as particle size. Adsorption-resistant coatings such as PEG are known to minimize molecular interactions and thereby increase circulation half life. Gref et al., Pharm Biotechnol 10:167-98, 1997. When chick embryos were co-injected with CPMV-AlexaFluor555 (red) and CPMV-PEG-FITC (green), the PEG coating completely eliminated the uptake by the cells in the blood vessel periphery (FIG. 5a).

Similarly, when adult mice were injected with CPMV-PEG-FITC, non-specific uptake by the liver and spleen was greatly reduced compared to those injected with CPMV-FITC (FIG. 5b). Based on quantification of CPMV fluorescence with digital image analysis, it was estimated that conjugation of PEG to the surface of CPMV reduced the accumulation in the liver and spleen by about 75%.

FIG. 5 shows CPMV uptake is eliminated in chick embryos and reduced significantly in adult mice by PEG coating. a. In a CPMV-AlexaFluor555 and CPMV-PEG-FITC co-perfused chick embryo, the same field of view shows uptake of CPMV-AlexaFluor555 (left) but not of CPMV-PEG-FITC (right). When CPMV-PEG-FITC was injected alone, similar results were obtained. b. FITC-labeled CPMV particles with (right) and without (left) a 3400 MW PEG coating were adjusted to an equal concentration by absorbance at 520 nm and injected into the tail vein of mice. Reticuloendothelial uptake was evaluated by fluorescence microscopy of liver and spleen tissue sections. These images are representative of three independent experiments.

### Example 7

Efficacy of CPMV-Based Nanoparticles as a Novel Platform for Sensitive In Vitro and In Vivo Cardiovascular Imaging

The present study demonstrated the efficacy of CPMV-based nanoparticles as a novel platform for sensitive in vitro and in vivo cardiovascular imaging. The biological and chemical properties of CPMV have been studied extensively and a number of unique features have been described that highlight its potential as an imaging sensor. Lin et al., Virology 265:20-34, 1999; Wang et al., Chem Biol 9:805-11, 200.; Porta et al., Virology 310:50-63, 2003.; Johnson et al., Annu Rev Phytopathol 35: 67-86, 1997, each incorporated herein by reference in its entirety. CPMV grows in the common black-eyed pea (cowpea) plant, and its production does not require sterile culture techniques or costly reagents such as culture medium or serum. Lin et al., Fold Des 1: 179-87, 1996. In addition, plant viruses themselves are non-pathogenic for humans. Brennan et al., Mol Biotechnol 17:15-26, 2001; Nicholas et al., Vaccine 20: 2727-34, 2002. CPMV particles are extremely stable and can withstand a variety of solvents and extremes of temperature and pH while retaining activity. Lomonossoff and Johnson, J. E. Prog Biophys Mol Biol 55:107-37, 1991. Because the viral genome is contained on two molecules of RNA that retain host infectivity without encapsidation, it can be manipulated at a genetic level to introduce desired mutations. Lin et al., Fold Des 1: 179-87, 1996. These particles are not limited to fluorescent labeling, and their highly multivalent properties may be exploited to display a wide variety of tags, including but not limited to radioactive isotopes, MRI contrast agents, or enzymatic moieties. Chatterji et al., Bioconjug Chem 15: 807-13, 2004, each incorporated herein by reference in their entirety.

When injected and visualized in living mouse and chick embryos, CPMV conjugated with the fluorescent dye AlexaFluor 555 provides a high signal-to-noise ratio with minimal fluorescence quenching and no apparent toxicity. The absence of fluorescence quenching may be attributed to the well defined sites of dye reactivity on the capsid surface, which provide sufficient steric separation of the dye molecules. Furthermore, similar results were achieved using a number of different commercially available fluorescent dyes such as AlexaFluor488 and fluorescein. The use of near-infrared fluorescent dyes such as Cy7 or AlexaFluor700/750 should enhance the quality of imaging at greater tissue depths in vivo by reducing the level of background autofluorescence and decreasing the likelihood of tissue damage during extended imaging studies.

Fluorescent CPMV particles were taken up by cells lining the vasculature and a subset of circulating blood cells in a time-dependent manner. In the chick CAM, the vascular uptake was restricted specifically to the venous system. This specificity, along with the bright signal, can be exploited to label the venous system, providing a convenient means to identify vascular origin and directionality within the tissue. In addition, uptake was also observed by the mononuclear phagocytes of the adult mouse reticuloendothelial system in the spleen and liver, resulting in the gradual clearance of freely circulating dye-conjugated CPMV particles in the experimental system. Using CPMV particles coated with 3400 Da PEG counteracted this phenomenon and significantly inhibited the uptake by cells of the vasculature and the circulating blood cells. These experiments indicate that the interaction of CPMV particles with the immune system may be modulated by increasing or decreasing the molecular weight and/or number of surface-conjugated PEG molecules. Raja, et al., Biomacromolecules 4: 472-6, 2003, incorporated herein by reference in its entirety.

The AlexaFluor555-conjugated CPMV nanoparticles proved particularly useful for the in vivo visualization of vasculature in mouse and chick embryos. Furthermore, in an in vivo model of tumor angiogenesis on the chick CAM, these fluorescent VNPs were superior to both fluorescent lectin and dextran for the visualization, identification, and quantification of vascularization in the tumor microenvironment.

The results suggest that these particles will be particularly well-suited to the visualization of rare molecular targets, due to their high per molecule signal. To demonstrate the general utility of CPMV-based imaging sensors, upright epifluorescence microscopy was utilized in this study. The combination of advanced imaging techniques such as two-photon confocal microscopy or selective plane illumination microscopy with these viral nanoparticles will maximize the sensitivity of these types of studies. Furthermore, multivalent display of vascular targeting peptides or proteins on the surface of CPMV would likely enhance their binding or targeting ability. The fact that these nanoparticles can be genetically modified to present novel peptide sequences opens the door for future targeted molecular bioimaging studies. Porta, C. et al., Virology 310: 50-63, 2003, incorporated herein by reference in its entirety.

### Example 8

Methodology

Propagation of CPMV in Plants. The primary leaves of cowpea seedlings were mechanically inoculated with 10 μg each of cDNA plasmids encoding RNA1 (pCP1) and RNA2 (pCP2). Dessens and Lomonossoff, J Gen Virol 74 (Pt 5): 889-92, 1993. The initial virus inoculum was extracted from infected cowpea leaves with 0.1 M potassium phosphate, pH 7.0 (phosphate buffer) 7 days post infection. Typically, 50 plants were infected with the plant extract, and the symptomatic leaves were harvested after three weeks. Virus was purified using standard techniques as previously described. Wang et al., Chem Biol 9:805-11, 2002, each incorporated herein by reference in its entirety.

Conjugation of CPMV with fluorescent dyes. AlexaFluor555 carboxylic acid, succinimidyl ester (Molecular Probes) was dissolved in DMSO and introduced at a ratio of 50/1 mol/mol into a solution of virus (1 mg/mL) so that the final solvent mixture was composed of 80% buffer and 20% DMSO (see FIG. 2a). In addition, CPMV particles were prepared using either fluorescein (FITC)-NHS (Molecular Probes) or FITC-NHS with a 3400 Da polyethylene glycol (PEG) spacer (Nektar Pharmaceuticals). After incubation at room temperature for 24 h, the conjugated virus was purified by ultracentrifugation through a sucrose gradient at 28,000 rpm, followed by resuspension in buffer PBS. The purity and fluorescence intensity of derivatized virus was determined by analytical size exclusion FPLC using a superose-6 column (FIG. 2b). Virus concentrations were determined by measuring the absorbance at 260 nm; virus at 0.1 mg/mL gives a standard absorbance of 0.8. The average molecular weight of the CPMV virion is 5.6×10⁶. Dye loading was obtained by measurement of absorbance at λₘₐₓ, with molar absorbtivity calibrated for each use by mixing known quantities of dye with CPMV (1 mg/mL) to correct for variations in dye purity or decomposition during storage.

Quantitation of in vitro fluorescence. 10 μl of a 100 μg/mL solution of fluorescent substrate was spotted on a glass slide and a coverslip was placed over top. Multiple fields were digitally captured by a Hammamatsu ORCA-ER 12 bit camera at 4×, 10×, and 20× magnification on the Zeiss Axioplan2 upright fluorescent microscope using the appropriate filter set. Image intensities were quantitated using the field intensity averaging function of the OpenLab acquisition software (Improvision Inc.), and the background fluorescence was subtracted. Data was collected and averaged over a minimum of five fields per objective per sample.

CPMV injections in adult mice. CPMV-AlexaFluor555 conjugate and/or FITC-dextran (10 kDa, Molecular Probes) (50 μg-1 mg in 0.1-0.2 mL PBS) were injected in the tail vein of CD-1 mice and allowed to circulate for 5 min up to 72 hrs. Tissues were fixed in 4% paraformaldehyde for 4 hours and frozen in Tissue Tek OCT embedding medium (Sakura Finetek) before sectioning. The 20 μm cryosections were mounted with Vectashield mounting medium (Vector Laboratories) before examination under a fluorescent microscope (Zeiss Axioplan2). In the experiments that utilized PEG-coated fluorescent CPMV, adult mice were injected in the tail vein with 250 μg or 500 μg of either CPMV-FITC or CPMV-PEG-FITC, and tissues were collected after 1 hr.

Injection, culture and imaging of mouse embryos. Injection of mouse embryos was performed at E9.5-E15.5 of development using a MM-33 micromanipulator (Fine Science Tools), microinjection needles drawn from glass pipettes, and a micro-adjustable syringe pump (Braintree Scientific). Embryos from timed matings (morning of vaginal plug counted as E0.5) were isolated with the yolk sac and placenta intact to preserve the embryonic vasculature and blood flow. Embryos were cultured in chamber slides using media as described. Jones et al., Genesis 34:228-35, 2002, incorporated herein by reference in its entirety. 10 μg of CPMV-AlexaFluor555 was injected through a small venule on the surface of the yolk sac, and whole embryos were visualized in vivo in culture media under the Axioplan2 fluorescent microscope.

Injection and fluorescent imaging of chick embryos. Fertilized White Leghorn chicken eggs were received from SPAFAS (North Franklin, Conn.) and incubated in a humidified incubator at 38° C. At day 4, eggshells were carefully removed, and embryos were incubated throughout the length of the experiment under shell-less conditions, in a covered dish placed in a humidified air incubator at 38° C. and 60% humidity. Zijlstra et al., J Biol Chem 279:27633-45, 2004, incorporated herein by reference in its entirety. Chick embryos at 10 days of development were injected with 50 μg of CPMV-AlexaFluor555 using a drawn glass capillary into a small venule in the CAM, and their extraembryonic vasculature was visualized with a Zeiss Axioplan2 upright microscope. For the PEG-coated fluorescent CPMV studies, embryos at 10 days of development were injected with 200 μl of a solution containing 50 μg of CPMV-PEG-FITC and 50 μg of CPMV-AlexaFluor555.

CAM tumor angiogenesis. Tumor onplants were generated by overlaying two gridded plastic meshes and embedding them into 30 μl of 2.2 mg/ml collagen. Seandel, M. et al., Blood 97: 2323-32, 2001, incorporated herein by reference in its entirety. Where indicated, HT1080 tumor cells were embedded in the collagen at 50,000 cells/onplant. Collagen onplants were placed on the chorioallantoic membrane of 10-day-old shell-less embryos. At day 7 after the placement of onplants, embryos were injected with 50 μg of CPMV-AlexaFluor555, rhodamine lectin (Lens culinaris agglutinin, Vector Labs), or 10 kDa FITC-dextran and visualized with a Zeiss Axioplan2 upright microscope. Tumors were excised, fixed in 4% paraformaldehyde, and sectioned.

### Example 9

Systemic Trafficking of Plant Virus Nanoparticles in Mice Via the Oral Route

The plant virus, cowpea mosaic virus (CPMV), is increasingly being used as a nanoparticle platform for multivalent display of peptides. A growing variety of applications have employed the CPMV display technology including vaccines, antiviral therapeutics, nanoblock chemistry, and materials science. CPMV chimeras can be inexpensively produced from experimentally infected cowpea plants and are completely stable at 37° C. and low pH, suggesting that they could be used as edible or mucosally-delivered vaccines or therapeutics. However, the fate of CPMV particles in vivo, or following delivery via the oral route, is unknown. To address this question, CPMV was examined in vitro and in vivo. CPMV was shown to be stable under simulated gastric conditions in vitro. The pattern of localization of CPMV particles to mouse tissues following oral or intravenous dosing was then determined. For several days following oral or intravenous inoculation, CPMV was found in a wide variety of tissues throughout the body, including the spleen, kidney, liver, lung, stomach, small intestine, lymph nodes, brain and bone marrow. CPMV particles were detected after cardiac perfusion, suggesting that the particles entered the tissues. This pattern was confirmed using methods to specifically detect the viral capsid proteins and the internal viral RNA. The stability of CPMV virions in the gastrointestinal tract followed by their systemic dissemination supports their use as orally bioavailable nanoparticles.

### Example 10

Materials and Methods

Preparation of purified cowpea mosaic virus (CPMV). cDNA clones of the CPMV genome were used to infect Kentucky cowpea (Vigna unguiculata) plants following which CPMV was purified from infected leaves by a method previously described. Dessens and Lomonossoff, J. Gen. Virol. 74:889-892, 1993; Khor et al., J. Virol. 76:4412-4419, 2002. The purification of intact CPMV particles was confirmed by analysis on an AKTA Explorer Superose™-6 size-exclusion column (Amersham Pharmacia). Virus samples suspended in 0.1 M phosphate buffer (pH 7.0) were applied to the column and following a wash with phosphate buffer, CPMV particles were eluted at a rate of 0.4 ml/min. TEM analyses were performed by depositing 20 μl aliquots of each sample onto 100-mesh carbon-coated copper grids for 2 minutes. The grids were then stained with 20 μl of 2% uranyl acetate and viewed with a Philips CM100 electron microscope.

Stability of CPMV particles in simulated gastric conditions. Simulated gastric fluid (SGF) and simulated intestinal fluid (SIF) were prepared according to Takagi et al. Takagi et al., Biol. Pharm. Bull 26:969-973, 2003. Briefly, pepsin (3.8 mg; Sigma) was dissolved into 5 ml of gastric control fluid (2 mg/ml NaCl, pH 2.0). SIF was prepared by dissolving pancreatin, (10 mg/ml; Sigma) in intestinal control fluid (0.05 M KH₂SO₄, pH 6.8). Both solutions were used within the same day. SGF (400 μl) was first incubated at 37° C. for 2 minutes before addition of CPMV (200 μg), giving a ratio of 10 U of pepsin activity/μg of CPMV. The tube contents were mixed by mild vortexing and the tube was immediately placed in a 37° C. water bath for time points ranging from 0-60 minutes, followed by neutralization with 70 μl of 200 mM sodium bicarbonate solution. Similarly, SIF was incubated at 37° C. for 2 minutes before addition of CPMV (200 μg) and the tube contents were placed in a 37° C. water bath for time points up to 120 minutes. Aliquots of virus exposed to SGF or SIF were mixed with 5 μl of NuPAGE 4× LDS-sample buffer, run on 4-12% SDS-PAGE gels, and stained with Simply Blue Safe Stain (Invitrogen). Aliquots of SGF- or SIF-treated virus containing serial 10-fold dilutions of CPMV ranging from 12.5 μg to 0.125 ng were also inoculated onto primary leaves of 7-day-old cowpea seedlings that had been dusted with carborundum. Five to seven days later, the leaves were observed for the presence of mosaic lesions.

Animals. All animals used in this study were 6-8 week old female C57 BL/6 mice obtained from The Scripps Research Institute Rodent Breeding Colony. Animals were used in compliance with IACUC approved protocols.

Isolation of RNA from mouse tissues following CPMV inoculation. Forty mice were inoculated with CPMV either by oral gavage or intravenous (i.v.) injection. Four groups of ten mice each were obtained and designated as follows: 1) Nine mice received 500 μg CPMV (5.37×10¹³ virus particles) each in 250 μl sterile, endotoxin-free PBS and one control sham-inoculated mouse received 250 μl sterile, endotoxin-free PBS by oral gavage. 2) Nine mice received 500 μg CPMV (5.37×10¹³ virus particles) each in 250 μl sterile, endotoxin-free PBS and one control sham-inoculated mouse received 250 μL sterile, endotoxin-free PBS by oral gavage. In addition, all were cardiac perfused at the time of sacrifice with sterile PBS after anesthetization with an intraperitoneal injection of chloral hydrate. 3) Nine mice received 100 μg CPMV (1.08×10¹³ virus particles) in 200 μl sterile, endotoxin-free PBS and one control sham-inoculated mouse received 200 μl sterile, endotoxin-free PBS by intravenous injection. 4) Nine mice received 100 μg CPMV (1.08×10¹³ virus particles) in 200 μl sterile, endotoxin-free PBS and one control sham-inoculated mouse received 200 μl sterile, endotoxin-free PBS by intravenous injection. In addition, all were cardiac perfused at the time of sacrifice with sterile PBS after anesthetization with an intraperitoneal injection of chloral hydrate. On day one post-inoculation, four mice per group including one sham-inoculated control, were either perfused or sacrificed by halothane depending on the designated group and portions of each of the following tissues were harvested, snap-frozen in liquid nitrogen, and stored at −80° C. for later RT-PCR analysis: spleen, kidney, liver, lung, stomach, duodenum, jejunum, ileum, brain and bone marrow. Three mice per each perfused and non-perfused group were euthanized on day 2 and day 3 post inoculation and the same tissues were collected and similarly stored for later RT-PCR analysis.

To examine the trafficking of CPMV using ingested, infected leaves, another group of seven mice was deprived of solid food for one day and each mouse was placed in a separate cage containing 1 g of CPMV-infected leaves (5 leaves) containing approximately 1 mg of CPMV (1.08×10¹⁴ CPMV particles). Each mouse ingested the entire gram of infected leaves within a day. Three mice were euthanized on day 1 post inoculation and two mice per day on days 2 and 3 post inoculation; tissues were isolated for RT-PCR as described above.

RT-PCR. Tissues isolated from mice were homogenized with a hand-held homogenizer (Omni International, Warrenton, Va.) in TRI reagent (MRC Inc, Cincinnati, Ohio) and RNA was then extracted according to the manufacturer's instructions. Following this, cDNA was synthesized using MMLV-RT and the downstream CPMV RNA 2 primer βBβCREV (5′ CGTATTCCAATTGTCATCACC 3′). The βBβCREV primer (60 pmol) was mixed with 500 ng of each tissue RNA and heated to 70° C. for 5 minutes, following which 20 units of RNAsin (Promega, Madison, Wis., USA), 4 mM each of dATP, dTTP, dCTP and dGTP (Roche, Mannheim, Germany), 20 units of MMLV-RT (Promega, Madison, Wis.) and MMLV-RT buffer were added. cDNA synthesis was carried out at 37° C. for 1.5 h followed by a 10 minute incubation at 70° C. to inactivate the enzyme. Double-stranded DNA was then amplified in a 100 μl reaction mix consisting of 5 μl of cDNA, 8 nmoles each of dATP, dTTP, dCTP and dGTP (Roche, Mannheim, Germany), 60 pmoles each of the upstream primer βBβCFOR (5′ GCACAAGGACCTGTTTGTGC 3′) and downstream primer βBβCREV (described above), 0.5 units of Taq polymerase (Roche, Mannheim, Germany), Taq polymerase buffer containing Mg (1.5 mM) supplied by the manufacturer, and purified water from Ambion (Austin, Tex.). Thirty cycles consisting of 1 minute denaturation at 95° C., 1 minute annealing at 55° C., and 1 minute extension at 72° C. were performed, resulting in a 150 bp PCR product. Five hundred ng of RNA extracted from CPMV-infected leaves, and purified water (Ambion), were used as positive and negative controls respectively for the RT-PCR reactions. All cDNA synthesis and PCR reactions were carried out in an MJ Research DNA Engine. The PCR products were analyzed on a 2% Seakem LE (BMA, Rockland, Me.) agarose gel alongside a 1 kb PLUS ladder (Invitrogen, San Diego, Calif.), and visualized with ethidium bromide on an AlphaImager 2200 MultiImage Light Cabinet (Alpha Innotech, San Leandro, Calif.). The sensitivity of the RT-PCR protocol was determined by setting up RT-PCR reactions with amounts of template CPMV RNA (purified from CPMV-infected cowpea leaves) ranging from 10⁰ to 10¹⁴ copies in increments of powers of 10. Employing the RT-PCR protocol described, it was possible to detect 10 copies of CPMV RNA purified from infected leaves.

Preparation and characterization of fluorescently conjugated CPMV. Oregon Green 488 (OG-488) carboxylic acid, succinimidyl ester fluorescent dye (Molecular Probes, Eugene, Oreg.) was conjugated to CPMV by mixing 22.9 mg of wild-type CPMV with OG-488 (200-fold excess relative to viral asymmetric subunit) in 11.5 mL of 0.1 M potassium phosphate buffer, pH 7.0, with gentle agitation at room temperature for 24 h. Initial separation of virus from unconjugated dye was accomplished by ultracentrifugation at 42,000 rpm over 3 ml of a 30% sucrose cushion. The pellet was then resuspended in 0.1M potassium phosphate and loaded on a 10-40% sucrose gradient for ultracentrifugation at 28,000 rpm for 3 hrs. The extracted bands were further purified by ultracentrifugation at 42,000 rpm for 3 hrs and the dye-labeled CPMV pellet was dissolved in 1 ml of sterile PBS (pH 7.0).

Inoculation of mice with Oregon Green 488-conjugated CPMV (OG-CPMV). For the determination of CPMV localization by fluorescence measurements, the same four groups of mice as described earlier were inoculated with OG-CPMV by either oral gavage with 500 μg OG-CPMV (5.37×10¹³ virus particles) each in 250 μl sterile, endotoxin-free PBS or i.v. injection with 100 μg OG-CPMV (1.08×10¹³ virus particles) in 200 μl sterile, endotoxin-free PBS and were either sacrificed by cardiac perfusion or euthanized by halothane depending on designated group. Four mice from each perfused or non-perfused group including one sham-inoculated control were sacrificed one day post inoculation and the following tissues were extracted, snap frozen in liquid nitrogen, weighed and stored at −20° C. for later fluorescence analysis: spleen, kidney, liver, lung, stomach, duodenum, jejunum, ileum, lymph nodes, and brain. Tissues from three mice per group on day 2 and day 3 post-inoculation were similarly harvested, weighed and stored.

Inoculation of mice with free Oregon Green 488 dye. Mice were administered 3.81 μg per mouse of free OG-488 dye by oral gavage, the equivalent amount of dye as that attached to the CPMV particles given by oral gavage. One mouse per day for three days post inoculation was perfused and tissues were isolated for fluorescence measurements. Similarly, by the intravenous route three mice were injected with 0.509 μg per mouse of free OG-488 dye, the equivalent amount of dye as that attached to the CPMV particles administered i.v. Tissues were harvested from mice following perfusion as described above.

Fluorescence measurements. Tissues were isolated and homogenized in PBS, then centrifuged at 10,000 g for 10 minutes at 4° C. to precipitate the cell debris. The OG-488-specific fluorescence emissions in the clarified tissue supernatants were determined using a Varian Cary Eclipse fluorescence spectrophotometer. A control sample of sham-inoculated tissue supernatant from each tissue was spiked with a known amount of OG-CPMV and the tissue-specific dye excitation and emission were determined for each tissue. The determined tissue specific emission λ value was then used in five separate scans of each tissue supernatant sample, following which the spectra were averaged and the emission value of the sham-inoculated control sample was subtracted as background.

Recovery of virus particles from mouse tissues. Four mice were orally inoculated with 150 μg/mouse of CPMV and 24 hours later, the mice were euthanized and the liver, spleen, and kidney tissues were extracted and pooled. Following homogenization of pooled tissue samples in PBS, the homogenates were centrifuged at 10,000 g for 10 minutes at 4° C., and the cleared tissue supernatants were removed to flat-bottom 50-ml centrifuge tubes, each containing a magnetic flea. Polyethylene glycol 8000 was added to each supernatant to a final concentration of 8% and the mixture was stirred for 30 minutes at 4° C. to precipitate any virus present. The resulting cloudy mixture was centrifuged at 6,000 rpm in a Sorvall RC5C centrifuge for 15 minutes and the pellet was resuspended in 50-500 μl of 0.1 M phosphate buffer (pH 7.0), depending on pellet size. For each tissue homogenate a 7-day-old cowpea plant was dusted with carborundum and the resuspended pellet from each tissue was rubbed onto a single primary leaf. Five to seven days later, the primary and secondary leaves were harvested and a circle of tissue weighing 70 mg was excised from each leaf. RNA was extracted using a Qiagen RNeasy Plant Mini Kit (Qiagen Research, Maryland, USA) and RT-PCR was conducted as described above. PCR products were electrophoresed on a 2% Seakem LE agarose gel. The PCR products were purified from the gel using a PCR purification kit (Qiagen) and sequenced (Retrogen) to confirm that they were CPMV specific. Southern hybridization of the products with a CPMV-specific probe was also performed using ³²P end-labeled oligonucleotides (Amersham Biosciences). PCR products were transfered to Hybond-N+ membrane (Amersham Biosciences) using standard techniques. Hybridization conditions were 3 hours at 55° C. and wash conditions were in 6×SSC/0.5% SDS and were performed twice for five minutes at room temperature and twice for 5 minutes at 50° C.

Infectivity of CPMV following incubation with blood. Whole blood samples were obtained from mice following cardiac puncture either with or without heparin. Samples were incubated at 4° C. overnight and then spun at 15,000 rpm for 10 minutes. Blood plasma or serum were recovered and incubated with CPMV (15 ug) in a one to one (v/v) ratio at 37° C. for 30 minutes while a control CPMV sample was incubated with PBS. Primary leaves from 7-day-old cowpea plants were then bruised with carborundum and inoculated with samples (at a concentration of 3 μg CPMV per leaf, or 300-fold more virus than is required to produce lesions) and observed daily for infection-induced mosaic symptoms.

### Example 11

Wild-Type CPMV Retains Stability and Infectivity Under Simulated Gastric Conditions

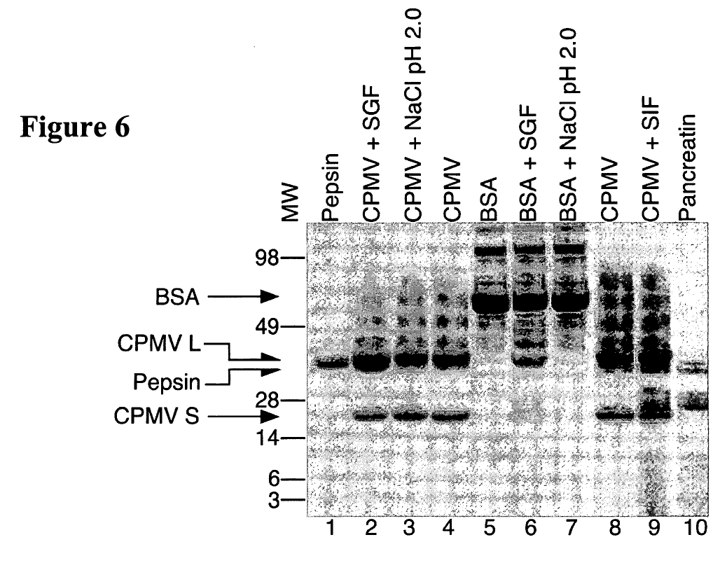

It has previously been noted that CPMV capsids are stable at acid pH and in low concentrations of pepsin. Xu et al., Dev Biol Stand 87:201-205, 1996. CPMV was studied to determine whether CPMV would remain stable and infectious following treatment in simulated gastric fluid (SGF) containing pepsin at pH 2.0, or in simulated intestinal fluid (SIF) containing pancreatin at pH 6.8. These in vitro conditions are typically used in pharmacokinetic or food science studies to evaluate the stability of proteins or formulations in the gastrointestinal environment. Takagi et al., Biol. Pharm. Bull 26:969-973, 2003. CPMV (200 μg) was incubated for varying times between 0-60 minutes in SGF or up to 120 minutes in SIF followed by removal of aliquots that were neutralized with bicarbonate. Visualization of SGF or SIF-treated CPMV on a Coomassie-stained gel showed that the L and S subunits remained intact throughout the time course with either treatment (FIG. 6). Pepsin (35 kD) is also visible in the samples (FIG. 6, lane 1). As a positive control, BSA (200 μg) was treated with SGF and proteolytic fragments appeared within 60 minutes of treatment (FIG. 6, Lane 6). To test the effects of simulated gastric conditions on CPMV infectivity, serial dilutions of CPMV that had been treated with SGF (60 minutes) or SIF (120 minutes) were inoculated onto 7-day old cowpea plants. At day 14 post-inoculation, the presence of lesions per sample of CPMV inoculated was noted. CPMV treated with SGF or SIF demonstrated infectivity similar to untreated CPMV. These results indicate that CPMV is resistant to simulated gastric or intestinal conditions and suggest that CPMV is likely to remain stable in the gastrointestinal tract in vivo.

FIG. 6 shows particle stability in SGF and SIF. SDS Page gel: CPMV following incubation with SGF for 60 minutes, SIF for 120 minutes, or acidic pH for 60 minutes. CPMV remains intact under acidic conditions and is resistant to pepsin and pancreatin degradation. BSA control confirms enzymatic activity.

### Example 12

CPMV Travels to a Variety of Tissues In Vivo

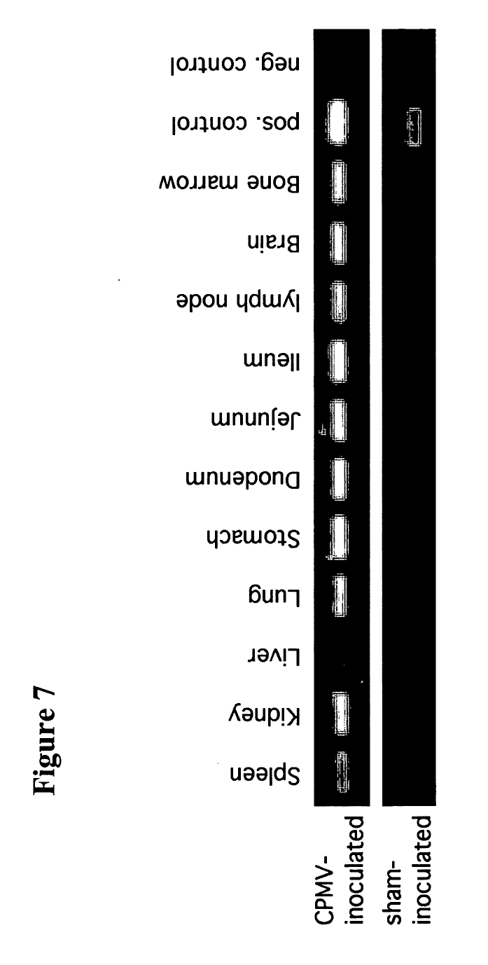

To study the tissue distribution of CPMV following different routes of delivery in the mouse, the presence of CPMV in mouse tissues was first determined by assaying for the CPMV genomic RNA by RT-PCR. It was reasoned that the single-stranded viral RNA would be protected from degradation in vivo when packaged inside intact CPMV capsids. Tissues were isolated from mice following CPMV administration by intravenous inoculation or by oral gavage. A duplicate group of inoculated animals underwent cardiac perfusion prior to tissue dissection to exclude virus that might be present in the bloodstream. RT-PCR products from a representative CPMV-inoculated mouse and sham (PBS)-inoculated control mouse injected i.v. at day 1 post inoculation are shown in FIG. 7. CPMV RNA was detected in all tissue examined of mice inoculated by i.v. injection in both perfused and non-perfused groups. Viral RNA persisted in tissues throughout days 2 and 3 post inoculation in the i.v. group (Table 1). No CPMV-specific PCR products were detected in sham-inoculated control mice (Table 1; FIG. 7). CPMV RNA was not detected in any tissues examined on either day 5 or day 7 post-inoculation.

FIG. 7 shows RT-PCR detection of CPMV RNA in mouse tissues. (A) One day following oral gavage with 500 μg of CPMV per mouse or (B) sham-inoculated. Positive controls: RNA purified from CPMV-infected cowpea leaves.

TABLE 1
Detection of CPMV RNA in tissues of mice inoculated i.v. with CPMV

| | Non-perfused | | | Perfused^{a} | | | |
|---|---|---|---|---|---|---|---|
| TISSUE^{c} | D1 | D2 | D3 | D1 | D2 | D3 | Ctrl^{b} |
| Spl | 3/3 | 3/3 | 3/3 | 3/3 | 3/3 | 3/3 | 0/3 |
| Kid | 3/3 | 3/3 | 3/3 | 3/3 | 3/3 | 3/3 | 0/3 |
| Liv | 2/3 | 3/3 | 3/3 | 3/3 | 3/3 | 3/3 | 0/3 |
| Lun | 3/3 | 3/3 | 3/3 | 3/3 | 3/3 | 3/3 | 0/3 |
| Brn | 3/3 | 3/3 | 3/3 | 3/3 | 2/3 | 3/3 | 0/3 |
| BM | 3/3 | 3/3 | 3/3 | 3/3 | 3/3 | 3/3 | 0/3 |
| Sto | 3/3 | 3/3 | 1/3 | 3/3 | 3/3 | 3/3 | 0/3 |
| Duo | 3/3 | 3/3 | 2/3 | 2/3 | 3/3 | 3/3 | 0/3 |
| Jej | 3/3 | 3/3 | 3/3 | 3/3 | 3/3 | 3/3 | 0/3 |
| Ile | 3/3 | 3/3 | 3/3 | 3/3 | 3/3 | 3/3 | 0/3 |

^{a}Perfused
^{b}Sham-inoculated
^{c}Tissue abbreviations: Spl: spleen; Kid: kidney; Liv: liver; Lun: lung; Brn: brain; BM: bone marrow; Sto: stomach; Duo: duodenum; Jej: jejunum; Ile: ileum;

For mice that received CPMV via the oral route, CPMV RNA was similarly detected in all tissues examined at day 1 post inoculation in both perfused and non-perfused groups (Table 2). CPMV RNA persisted in kidney, liver, lung, bone marrow and brain in the orally-inoculated group, as well as in the gastrointestinal tract. Within the GI tract, there was less signal at day 2 and day 3 in perfused animals, suggesting that in these tissues, much of the virus detected is in the blood. The presence of CPMV in the blood following oral gavage was also investigated. CPMV RNA was detected in the blood of all orally gavaged mice at 4 hours (n=5), 1 day (n=5), 2 days (n=4), and 3 days (n=5) post-inoculation. These results indicate that CPMV was systemically distributed in mice via the vasculature to either bind the endothelium or enter the tissue parenchyma in a variety of tissues following oral or i.v. inoculation.

TABLE 2
Detection of CPMV RNA in tissues of mice inoculated orally with CPMV

| | Non-perfused | | | Perfused^{a} | | | |
|---|---|---|---|---|---|---|---|
| TISSUE^{c} | D1 | D2 | D3 | D1 | D2 | D3 | Ctrl^{b} |
| Spl | 3/3 | 1/3 | 0/3 | 3/3 | 0/3 | 0/3 | 0/3 |
| Kid | 3/3 | 0/3 | 1/3 | 3/3 | 3/3 | 2/3 | 0/3 |
| Liv | 3/3 | 3/3 | 2/2 | 3/3 | 3/3 | 3/3 | 0/3 |
| Lun | 3/3 | 1/3 | 1/3 | 3/3 | 2/3 | 1/3 | 0/3 |
| Brn | 3/3 | 1/3 | 3/3 | 3/3 | 1/3 | 2/3 | 0/3 |
| BM | 3/3 | 0/3 | 1/3 | 2/3 | 0/3 | 0/3 | 0/3 |
| Sto | 3/3 | 1/3 | 3/3 | 2/3 | 1/3 | 0/3 | 0/3 |
| Duo | 3/3 | 1/3 | 2/3 | 3/3 | 1/3 | 0/2 | 0/3 |
| Jej | 3/3 | 0/3 | 2/3 | 3/3 | 1/3 | 0/2 | 0/3 |
| Ile | 3/3 | 3/3 | 2/3 | 3/3 | 1/3 | 1/3 | 0/3 |

^{a}Perfused
^{b}Sham-ininoculated
^{c}Tissue abbreviations: Spl: spleen; Kid: kidney; Liv: liver; Lun: lung; Brn: brain; BM: bone marrow; Sto: stomach; Duo: duodenum; Jej: jejunum; Ile: ileum;

The oral gavage procedure introduces an inoculum directly into the stomach with a possibility of trauma to the esophagus or stomach epithelium allowing virus to directly enter the circulation. To determine if the systemic trafficking of CPMV observed was a result of the gavage procedure, mice were allowed to ingest 1 g of CPMV-infected cowpea leaves, which contained approximately 1 mg of CPMV (1.08×10¹⁴ virus particles). Three mice on day 1 post-ingestion and two mice each on days 2 and 3 post-ingestion were euthanized and the same tissues extracted as from the orally-gavaged mice. The proportion of mice that were positive for CPMV RNA on days 1, 2 and 3 was determined for each tissue. Similar to the mice receiving CPMV by oral gavage, on day 1 post ingestion, a systemic distribution of CPMV was apparent, with CPMV RNA being detected in the spleen, liver, lungs, stomach, ileum and bone marrow in 2 out of 3 mice, as well as in the kidney, duodenum, jejunum and brain in 1 out of 3 mice. These results are consistent with the initial pattern of tissue distribution of CPMV particles observed following oral gavage with purified CPMV. The RT-PCR results indicate that the ability of CPMV to access the systemic circulation following oral administration is a natural consequence of eating infected leaves and is not an artifact of the gavage procedure. In contrast to animals receiving purified CPMV by gavage, CPMV RNA did not persist in tissues past the first day following ingestion of infected leaves. This may reflect differences in the accessbility to the gut epithelial lining of purified viruses versus viruses possibly bound to other proteins within infected leaves. Alternatively there may be less virus available to reach the circulation when it is delivered via leaves.

### Example 13

Fluorescent Labeling of CPMV to Track Viruses In Vivo

The above experiments measured RNA packaged inside virus particles as an indication of the presence of CPMV in tissues. It is possible, however, that free RNA had been delivered to tissues in the absence of intact particles. To confirm that particles were being detected, a complementary study was performed in which the trafficking of CPMV particles labeled with a fluorescent dye was followed in mice. The CPMV capsid has five reactive lysine residues on each asymmetric unit, with one residue on the small subunit, Lys 38, having the highest reactivity. Chatterji et al., Chem Biol 11: 855-63, 2004b; Wang et al., Chem. Biol. 9:805-11, 2002a. The surface lysine residues have been successfully conjugated to chemicals such as dye molecules and proteins. Chatterji et al., Bioconjug. Chem. 15: 807-13, 2004a; Wang et al., Chem. Biol. 9:805-11, 2002a; Wang et al., Angew. Chem. Int. Ed. 41:459-462, 2002c. After testing several different fluorophores for the most sensitive detection in the widest variety of tissues, these reactive lysines were used to conjugate the NHS ester of the Oregon Green-488 fluorophore onto the CPMV particle. Dilutions of free OG-488 dye were prepared in phosphate-buffered saline and the fluorescence emissions were detected using a fluorescence spectrophotometer (see Materials and Methods). The standard curve of OG-488 dye concentration versus fluorescence emission intensities was plotted. Each data point was the average of values obtained from three independent parallel measurements, with a standard deviation of 1% between measurements. The fluorescence emission intensity of OG-488 dye conjugated to a known concentration of CPMV was measured and the concentration of OG-488 dye conjugated to CPMV was determined from the standard curve. Using this method the dye:particle ratio was found to be 130 dyes per particle for the stock of OG-CPMV.

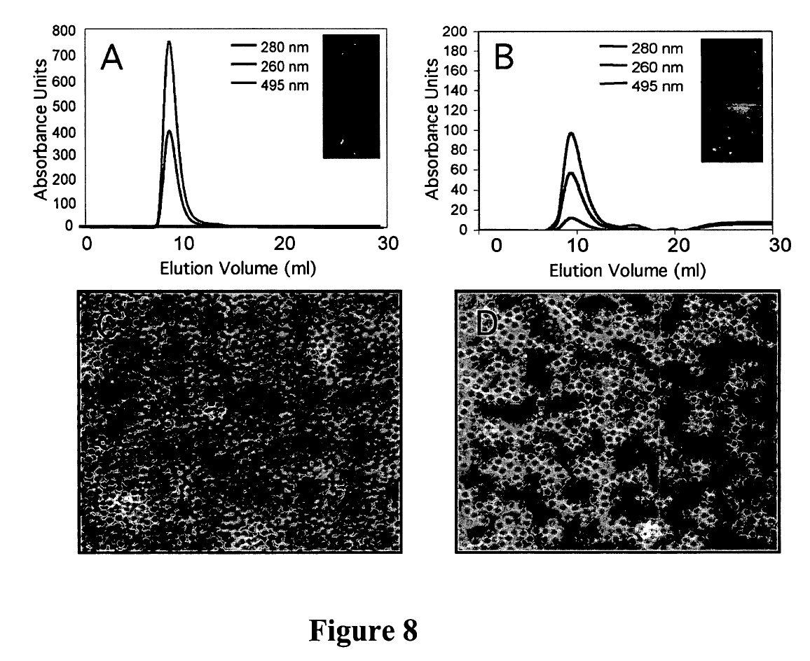

The integrity of the fluorescent viruses was monitored by three different methods: sucrose gradient centrifugation, size exclusion chromatography, and transmission electron microscopy (TEM) (FIG. 8). OG-CPMV demonstrated unique excitation at 494 nm, confirming that the OG fluorophore was indeed attached to the CPMV particles (FIGS. 8A and 8B). In the sucrose gradient, the OG-CPMV virus suspension separated into distinct bands of intact particles that fluoresced under UV light (FIG. 8B versus the non-fluorescent wild-type CPMV bands in FIG. 8A). The upper and lower virus bands separated by sucrose gradient correspond to intact CPMV middle- and bottom-component particles encapsidating either RNA-2 or RNA-1, respectively. Intact virions were detected by size exclusion chromatography on a Superose-6 column with retention times of approximately 25 minutes at an elution rate of 0.4 ml/min (FIGS. 8A and 8B). In contrast, broken particles and individual subunit proteins, which typically elute from the column after 50-60 minutes, were not detected in significant amounts. The intact nature of both wild-type CPMV and OG-CPMV was confirmed by transmission electron microscopy (FIGS. 8C and 8D).

FIG. 8 shows characterization of Oregon Green-conjugated CPMV (OG-CPMV) particles. Conjugation of the NHS ester of fluorescent dye Oregon Green-488 (OG-488) to reactive lysines on the asymmetric unit of the CPMV capsid was performed to produce the dye-conjugated Oregon Green-CPMV (OG-CPMV). Wild-type CPMV (WT CPMV) and OG-CPMV were analyzed by size-exclusion chromatography (panels A and B respectively), sucrose gradient sedimentation (insets in panels A and B) and transmission electron microscopy (panels C and D).

### Example 14

Trafficking of OG-CPMV In Vivo

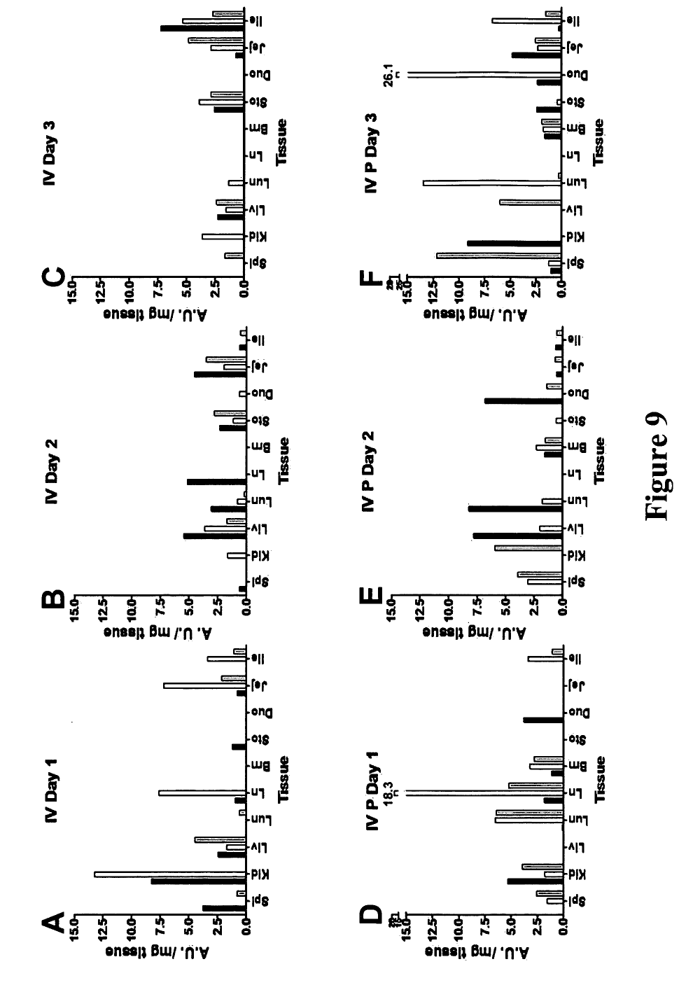

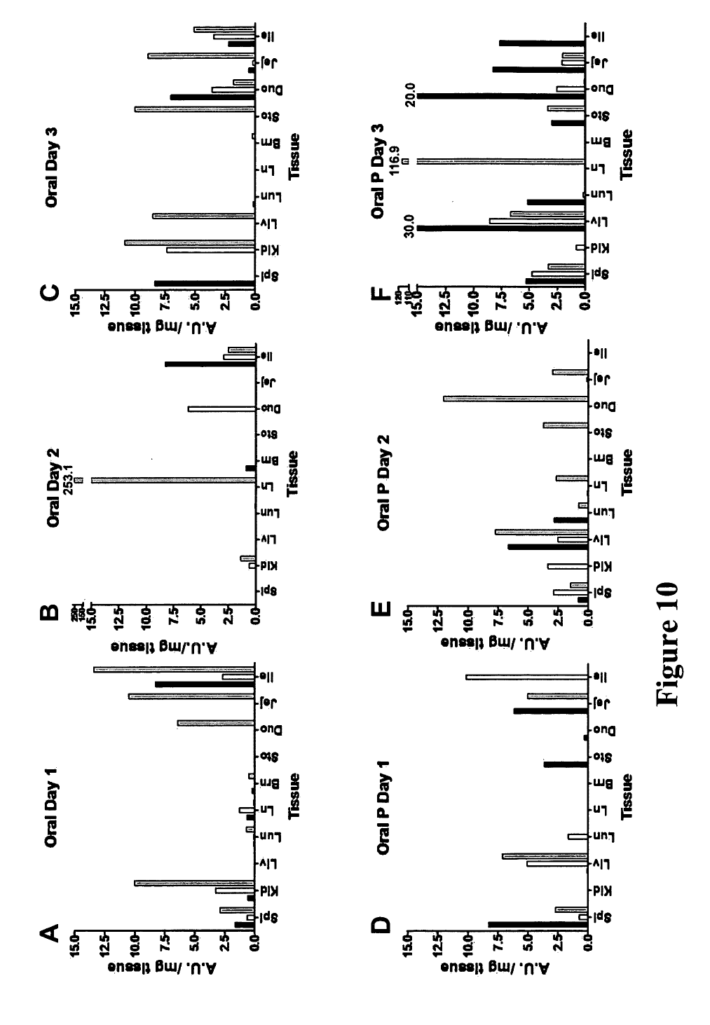

To measure the trafficking of OG-CPMV in vivo, mice (3/group) were injected intravenously with 100 μg per mouse of OG-CPMV. At 1, 2, and 3 days post-injection a duplicate set of animals was perfused, tissues were harvested from both sets and the OG-specific fluorescent signal measured for each tissue (FIG. 9). OG-CPMV was detected in the following tissues: spleen, kidney, liver, lung, stomach, duodenum, jejunum, ileum, lymph nodes, and brain. Some variation in the presence and quantity of OG-CPMV was observed. These results confirm that CPMV enters the tissues from the vascular system and support the tissue distribution of CPMV following i.v. administration that was observed by RT-PCR (Table 1). To determine whether OG-CPMV could be detected in tissues following oral administration, mice (3/group) were administered 500 μg OG-CPMV by oral gavage and one set received cardiac perfusion with saline at the time of tissue harvest. Again, OG-CPMV was detected in each tissue but with some variation among mice (FIG. 10). At days 1 and 2, fluorescence was detected in most tissues at lower levels than the i.v. groups but at higher levels at day 3. Although CPMV particles themselves were stable in the simulated gastrointestinal environment, an experiment evaluating the stability of OG-CPMV in SGF showed that there was 20-40% removal of the dye from the particles after a 60 minute incubation, indicating that the particle-dye linkage might be exposed on the particle surface and thus more susceptible to pepsin cleavage than the rest of the particle. To determine whether free dye might be contributing to the observed localization in tissues, a control experiment inoculating animals orally with free dye was also performed to ask whether dye might be detached from the particles in the gastrointestinal tract. The fluorescence due to free OG-488 dye was negligible in all tissues at all time points with the exception of the gastrointestinal tissues and the mesenteric and cervical lymph nodes in 1 of 3 mice. Together with the PCR results these data suggest that the plant virus CPMV enters the mammalian systemic circulation following oral administration and enters the tissue parenchyma in a variety of tissues.

FIG. 9 shows systemic trafficking in mice inoculated intravenously with OG-CPMV. OG-CPMV fluorescence detected (Absorbance Units (A.U.) per mg tissue) in tissues harvested from three individual mice (black, grey and white bars respectively) receiving OG-CPMV intravenously. (A): Day 1: (B): Day 2, (C): Day 3, (D): Day 1 saline-perfused, (E): Day 2 saline-perfused, (F): Day 3 saline-perfused post-inoculation.

FIG. 10 shows systemic trafficking in mice inoculated orally with OG-CPMV. OG-CPMV fluorescence detected (A.U. per mg tissue) in tissues harvested from three individual mice (black, grey and white bars respectively) receiving OG-CPMV by oral gavage. (A): Day 1: (B): Day 2, (C): Day 3, (D): Day 1 saline-perfused, (E): Day 2 saline-perfused, (F): Day 3 saline-perfused post-inoculation.

The fluorescence measurements provide a useful means to compare relative amounts of CPMV particles between tissues of mice in different treatment groups. There was higher mouse-to-mouse variation in OG-488 fluorescence emission in the various tissues following oral administration than intravenous administration, suggesting that the efficiency of uptake of CPMV in the gastrointestinal tract is variable in individual mice or that variable amounts of degradation of the fluorescent dye occurs in the gastrointestinal tract, but overall the distribution patterns of CPMV particles in both i.v.-inoculated and orally-inoculated mice were similar.

### Example 15

Recovery of CPMV Particles from Mouse Tissues

The results of both the RT-PCR and fluorescence experiments suggested that intact CPMV particles were being recovered from tissues. To determine whether the recovered virus particles were infectious to plants, tissue homogenates from liver and spleen combined from several animals were PEG-precipitated and inoculated onto cowpea seedlings. Five to seven days post-inoculation, RNA was extracted from the treated leaves and from secondary leaves, and CPMV-specific RT-PCR was performed. A control experiment titrating the appearance of symptoms and RT-PCR products showed that 10 ng of inoculated CPMV was required to observe symptoms and 1 ng for a PCR signal, respectively. No symptoms were observed on the inoculated primary leaves or on secondary leaves. While faint RT-PCR bands were detected from primary leaves, indicating the presence of viral RNA in the inoculum as previously demonstrated, CPMV-specific PCR products were not detected in secondary leaves suggesting that replication and spread within the plant did not occur. These experiments were performed several times with similar results. These data suggest that either the amount of CPMV recovered from tissues is too low to initiate an infectious cycle in plants, or that the virus particles that are recovered are somehow inactivated.

### Example 16

Infectivity of CPMV is Reduced Following Incubation with Murine Blood

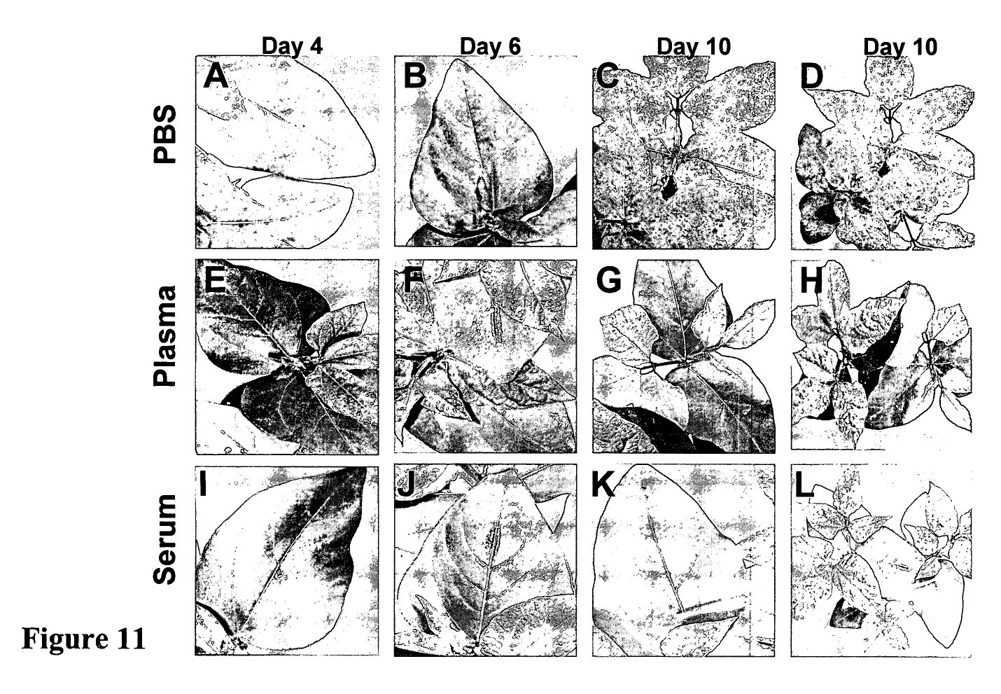

Since it was demonstrated in vitro that CPMV is resistant to simulated conditions of the stomach and gastrointestinal tract (FIG. 6), it was hypothesized that the virus is inactivated following oral or i.v. inoculation by a separate and independent mechanism in the circulation. To investigate whether components of the blood are responsible for inhibition of virus infectivity, CPMV was incubated with either plasma or serum for 30 minutes at 37° C. and inoculated into leaves of 7-day-old cowpea plants at a concentration 300-fold greater than that required to produce symptoms. Inoculated plants were observed daily for the appearance of lesions. By day 4 post inoculation, a control infection showed very strong signs of infection with an abundance of distinct symptoms on all primary leaves, while plants inoculated with plasma- or serum-incubated virus showed almost no signs of infection (FIG. 11). By day 7, infection was observed in most plants but at distinct levels of infection among different groups. Plants infected with plasma-incubated virus produced an average of 6 lesions per cm on primary leaves, while those infected with serum-incubated virus produced less than 1 lesion per cm² and the control infection showed too many lesions to count in all primary leaves. This experiment suggests that blood components found in the plasma and serum significantly inhibit the infectivity of CPMV in plants.

FIG. 11 shows inactivation of CPMV infectivity by murine serum and plasma. Cowpea leaves inoculated with CPMV that had been incubated with either PBS (A-D), mouse plasma (E-H), or mouse serum (I-L). Presence of typical mosaic symptoms on leaves was noted at various times post-infection as indicated.

### Example 17

Plant Virus CPMV Shows a Natural Bioavailability in Mammals

This study demonstrates that the plant virus CPMV shows a natural bioavailability in mammals. CPMV may be delivered orally, transported across the intestinal epithelia of mice, and translocated to a variety of tissues in vivo. The pattern of CPMV localization in mice was ascertained by two independent methods that suggest CPMV particles disseminate systemically from the gastrointestinal tract via the blood. Virus particles were present in the systemic circulation as well as the tissue parenchyma. These results confirm that the biophysical characteristics of CPMV nanoparticles, including stability at low pH, size, and resistance to proteolysis, renders them able to traffic into the systemic circulation from the gastrointestinal tract.

The general similarity in CPMV distribution patterns between perfused and non-perfused mice indicates that CPMV particles are present not only in the vasculature, but that some particles bind to endothelial cells or enter the tissue parenchyma as well. Interestingly this phenomenon was seen in brain. The presence of CPMV was noted in brain in perfused animals by the PCR method regardless of the route of inoculation (Tables 2 and 3). Using OG-CPMV however, CPMV was observed only in the perfused brain following intravenous administration, suggesting that the specific fluorescence in the brain endothelium or parenchyma is higher when the blood is removed (FIG. 9), and that when introduced by the oral route less virus may reach the brain. It is possible, however, that a small amount of CPMV gains access to the brain parenchyma. The picornaviruses poliovirus and coxsackievirus are able to enter the central nervous system from peripheral enteric sites by axonal transport and such access appears to be independent of known virus receptors. Feuer et al., Am. J. Pathol. 163:1379-1393, 2003; Ohka et al., Virology 250:67-75, 1998; Yang et al., Virology 229:421-428, 1997. The results suggest that if neuronal access is occurring the transport may be a feature of capsid structures in the picornavirus superfamily and the ability to interact with the neuronal architecture. Alternatively it is possible that CPMV may be present within endothelial cells in the brain. Preliminary studies suggest that vascular endothelium internalizes CPMV in vivo (Lewis et al., submitted). In addition, preliminary studies indicate a specific interaction between the CPMV capsid and mammalian proteins, and this interaction may coat or inactivate the CPMV capsid so that it is non-infectious for plants. Sensitive pharmacokinetic studies using radiolabeled CPMV particles will be an appropriate way to further quantify the bioavailability, half-life, and tissue accessibility of CPMV particles, particularly for the brain, and these studies are in progress.

One consideration when using a proteinaceous nanoparticle is the potential for immune responses against the protein coat. CPMV has been exploited as a nanoparticle platform for presenting immunogenic epitopes for vaccine development, thus it is known that the nanoparticle itself can be immunogenic (Raja et al., 2003), although immune stimulation via the oral route is negligible in mice. Raja et al., Biomacromolecules 4:472-6, 2003; Brennan et al., J. Virol 73: 930-938, 1999a; Durrani et al., J. Immunol. Meth. 220:93-103, 1998. Preliminary studies indicate that preexisting humoral immunity to CPMV does not impede oral trafficking. In addition, it has been shown that it is possible to modulate the anti-CPMV antibody response if desired by chemically conjugating particles to polyethylene glycol (PEG). Raja et al., Biomacromolecules 4:472-6, 2003. Further, recent studies have indicated that existing anti-nanoparticle responses do not inhibit the subsequent development of epitope-specific responses to antigenic peptides on viruses or virus-like particles. Mandl et al., J. Virol. 75:622-627, 2001.; Ruedl et al., Journal of Virology 79:717-724, 2005. Interestingly, ingestion of black-eyed peas has long been a preventative practice for measles virus (MV) infection in certain parts of Africa, and it has recently been shown that CPMV and MV share an antibody epitope. Olszewska and Steward, Virology 310:183-189, 2003. These studies support the idea that oral delivery of an immunogen as part of the CPMV nanoparticle is likely to be a feasible vaccination strategy if sufficient immunity is induced. Together these studies provide further evidence for the feasibility of “biofarming” vaccines by producing viral nanoparticle-based immunogens in plants. Olszewska and Steward, Virology 310:183-189, 2003.

This use of CPMV nanoparticles represents a novel strategy for oral delivery of therapeutics. It is not known how CPMV crosses the intestinal epithelium, however, uptake of other orally-delivered particulates in the intestine is mainly focused in the Peyer's patches (PP) of the small and large intestine. PP are intestinal protrusions of lymphoid aggregates or follicles on the anti-mesenteric side of the gut wall and are covered by a monostratified epithelial layer termed the follicle-associated epithelium or FAE. Hussain et al., Advanced Drug Delivery Reviews 50:107-142, 2001; Kerneis and Pringault, Seminars in Immunology 11:205-215, 1999. Specialized epithelial cells called M (or membranous) cells are scattered throughout the FAE and are important in sampling particulate antigens in the gut lumen and passing them across the epithelium. Gebert et al., International Review of Cytology 167:91-159, 1996; Neutra et al., Cell 86:345-348, 1996; Neutra et al., Seminars in Immunology 11:171-181, 1999. M cells are capable of internalizing particulates in a broad size range from 28 nm to several microns. Beier and Gebert, American Journal of Physiology 275:G130-G137, 1998; Jani et al., International Journal of Pharmaceutics 105:157-168, 1994; Neutra et al., Seminars in Immunology 11:171-181, 1999; O'Hagan, Adv. Drug Deliv. Reviews 5:265-285, 1990. Peyer's patches appear to take up particles of varying hydrophobicity, but the size and surface charge of particles seem to be factors in their ability to passage through the mucus layer overlying the intestinal epithelia to contact M cells and intestinal enterocytes. Beier and Gebert, American Journal of Physiology 275:G130-G137, 1998; Hussain et al., Advanced Drug Delivery Reviews 50:107-142, 2001. For example, cationic dyes in the nanometer size range tend to get trapped in the negatively charged mucus, while negatively charged carboxylated fluorescent latex nanoparticles of similar size have been shown to permeate the mucus layer. However, strongly negatively charged particles may be repelled from the mucus layer and carried away from M cells by mucus flow. Szentkuti et al., J. Controlled Release 46:233-242, 1997. Poliovirus with its analogous capsid structure to CPMV is known to be transcytosed by M cells in the intestine. Neutra et al., Cell 86:345-348, 1996; Neutra et al., Seminars in Immunology 11: 171-181, 1999; Ohka et al., Virology 250:67-75, 1998; Ouzilou et al., J. Gen. Virol. 83:2177-2182, 2002. Based on the particle size and biochemical characteristics it seems likely that CPMV is taken up by M-cells in Peyer's patches and this possibility is under investigation. Preliminary studies indicate that CPMV co-localizes with M-cells in the epithelium of the mouse ileum and is deposited in the underlying Peyer's patch lymphoid tissue.

These results also suggest that systemic exposure to plant viruses naturally found in food sources is likely to be a frequent occurrence. For example, the host plant for CPMV, Vigna unguiculata or black-eyed bean, is a food source in parts of Africa and South America and the virus is found in high concentrations both in the leaves and in the beans. Olszewska and Steward, Virology 310:183-189, 2003. The consequences of ingestion and systemic exposure to plant viruses is unknown, but may be a heretofore unrecognized source of antigen exposure. Although it is a member of the picomavirus superfamily, the host range of CPMV is restricted to plants with no evidence that replication of CPMV occurs in mice. Nevertheless, there are examples of viral host ranges spanning the plant and animal kingdoms (Selling 1990, Dasgupta 2003). These findings that CPMV nanoparticles remain intact following in vivo administration, combined with their structural relationship to picornaviruses of mammals, suggest a possible opportunity for inter-kingdom transmission of picorna-like viruses from plants to animals on an evolutionary time scale. Hendrix, Current Biology 9:R914-R917, 1999. Of course, this raises safety considerations for working with plant viruses in the laboratory, and it has been shown that CPMV may be inactivated by UV irradiation. Langeveld et al., Vaccine 19:3661-70, 2001. Importantly, these studies also show that CPMV is bioavailable when administered in an edible form in cowpea leaves. A similar distribution of CPMV RNA was observed in mice that ingested CPMV in the form of CPMV-infected leaves as that observed in mice that received CPMV by oral gavage, except at lower levels. This discrepancy could be due to fewer CPMV particles being available from the infected leaves in comparison to purified virus because the liberation of CPMV particles from the leaves during chewing and digestion is likely to be less efficient than mechanical purification. Importantly, the similarity in tissue distribution between the two routes indicates that the systemic dissemination of CPMV induced by oral inoculation is due to particle uptake by the intestinal epithelia and subsequent passage into the general circulation, and not to entry of virus particles into the circulation as a result of trauma to the GI tract by the oral gavage procedure. In addition, the results suggest that CPMV-based edible therapeutics or vaccines are feasible and that further purification of viruses from infected plant tissue may not be necessary. This is especially significant since CPMV particle stability was demonstrated under relatively natural conditions of ingestion, in contrast to some other nanoparticle systems that use more artificial routes of administration such as direct injection into the intestine or neutralization of stomach acidity to bypass the degradative and acidic environment of the stomach, pancreatic lipases, and bile digestive enzymes of the duodenum. Damge et al., J. Pharm. Pharmacol. 52: 1049-1056, 2000; Wells et al., Infection and Immunity 56:278-282, 1998. This makes it possible to generate edible vaccines or therapeutics for use in developing countries. Finally, the favorable in vivo characteristics of CPMV nanoparticles support their further development for applications such as drug delivery.

All publications and patent applications cited in this specification are herein incorporated by reference in their entirety for all purposes as if each individual publication or patent application were specifically and individually indicated to be incorporated by reference for all purposes.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

### Example 18

CPMV Uptake by Antigen Presenting Cells and CTL Mediated Vaccine Protection

The use of plants and plant viruses for producing vaccines holds enormous potential for large-scale global vaccine development. Cowpea mosaic virus (CPMV) is a plant pathogen that has been established as a nanoparticle platform for displaying antigens for inducing humoral immunity. Here it was investigated whether CPMV could be used to display antigens to induce T-cell immunity. Antigen presenting cells, both cell lines and primary dendritic cells derived from bone marrow, were able to bind and internalize CPMV particles in vitro. Macrophages, CD8α⁺ dendritic cells, B cells and natural killer cells were also able to internalize CPMV particles in vivo. To test the induction of MHC-I restricted CD8+ responses, immunodominant T-cell epitopes derived from lymphocytic choriomeningitis virus (LCMV) were introduced into a CPMV infectious clone encoding the CPMV capsid subunits, resulting in the multivalent presentation of 60 copies of the epitopes on each CPMV particle. CD8 cells from C57BL/6 mice that were intraperitoneally immunized with CPMV displaying the immunodominant GP33 epitope produced a GP33-specific IFN-γ and TNF-α response after a single immunization and without adjuvant. Moreover, immunization with the CPMV-GP33 chimera protected 50% of the mice challenged intracranially with a lethal LCMV dose. This study indicates that use of the CPMV system can be expanded to include induction of cytotoxic T-cells (CTL) for vaccine purposes.

### Example 19

Materials and Methods

Mice. Adult female C57BL/6 (H-2b) mice were obtained from the Scripps Research Institute animal facility and housed in specific pathogen-free conditions according to Institutional Animal Care and Use Committee (IACUC) guidelines.

Cells and Viruses. MC57 (H-2b) fibroblast cells were grown in RPMI-1640 supplemented with 7% fetal bovine serum (FBS), 2 mM L-glutamine, 100 U/ml penicillin G, and 100 μg/ml streptomycin (all from Gibco-BRL, Rockville, Md.). Balb C17 (H-2d) fibroblasts were grown in minimum essential media (MEM) with the above supplements. Bone-marrow derived dendritic cells were isolated and cultured as described. Hahm et al., Virology 323:292, 2004.

Lymphocytic choriomeningitis virus (LCMV), Armstrong strain, was prepared by a single passage in BHK-21 cells grown in medium RPMI with the above supplements. LCMV titers in plaque forming units (PFU) and 50% lethal doses (LD₅₀) were determined by plaque assay on Vero cells and in C57Bl/6 animals, respectively, according to standard procedures. Dutko and Oldstone. J. Gen. Virol. 64:1689, 1983; von Herrath and Whitton. Curr. Protocol. Immun. 19.10.1, 2000.

Chemical coupling of fluorescein and AlexaFluor 488 dyes to CPMV. Fluorescein-5-maleimide dye (Molecular Probes) was coupled to cysteines on the coat protein of the CPMV-vEFα chimera (a gift from Dr. J. Johnson) as previously described with the following modifications. Wang et al., Chem. Biol. 9:813, 2002. The dye (2.28 mg) was resupended in DMSO and mixed with 5 mg of CPMV in potassium phosphate buffer (molar ratio of 100 dyes per asymmetric large-small CPMV coat protein unit in 5 ml final volume) and incubated 72 hours at 4° C. To conjugate dyes to lysines on wild-type CPMV capsid, 1 mg Alexa fluor 488 carboxylic acid, succinimidyl ester (Molecular Probes) was resuspended in 0.1 M K-phosphate buffer and mixed with 5 mg of CPMV-wt in a total volume of 1 ml of the same buffer using a molar ratio of 30 dyes per asymmetric large-small coat protein unit. The virus-dye suspension was incubated at room temperature in a rolling shaker for 72 hours. After incubation the samples were initially purified by ultracentrifugation at 42,000 rpm (3 hours, 4° C.) and resuspended in 1 ml of the same buffer. To eliminate free dye the sample was further purified by a sucrose gradient (30%-10%) ultracentrifugation at 28,000 rpm (2 hours, 4° C.). After collecting the labeled CPMV fraction the virus was concentrated by ultracentrifugation at 42,000 rpm (3 hours, 4° C.). The final pellet was resuspended in PBS (Gibco-BRL) and filtered through a 0.2-μm membrane (Costar) to eliminate aggregate particles. The virus concentration was calculated as described above. The dye concentration was obtained measuring the absorbance of the sample at 495 and using the molar extinction coefficient (E) of the dye. The number of dyes per virus particle obtained was 71.33 for AF488 and 23 for fluorescein, where dyes/particle=Abs₄₉₅×dilution×MW of CPMV/ε×g of CPMV, and CPMV MW=5.6×10⁶ g/mol.

Cellular uptake of CPMV in vitro. MC 57 and Balb Cl 7 cells were cultured in Lab-Tek II chamber polylysine slides (Nalge Nunc Naperville, Fla.) and CPMV-Fluorescein (35 μg/ml) was added to the media. DCs were cultured in 24 well plates with CPMV-AF488 (20 μg/ml). Cultures were incubated overnight at 37° C. with 5% CO₂. Cells were washed 3 times with PBS, fixed with 2% formaldehyde, stained with DAPI or Hoechst and visualized with a Zeiss Axiovert S100 immunofluorescent microscope. DC were also stained with specific antibodies using the following reagents: R-PE conjugated anti-CD86 (B7.2; clone GL1), R-PE conjugated anti-CD80 (B7.1; clone 16-10A1, both from BD PharMingen) and allophycocyanin (APC) anti-CD11c (clone N418; eBioscience Inc.). Cells were acquired on a FACSort flow cytometer (30,000 events per sample) and analyzed with FlowJo software (Treestar, San Carlos, Calif.).

Cellular uptake of CPMV in vivo. Groups of 3 mice were inoculated i.v. or i.p. with 100 μg of CPMV-AF488 and negative control mice were inoculated with PBS. After four hours mice were sacrificed, and the spleens were harvested. The spleens were injected with 1 ml solution of 1 mg/ml of Collagenase-D (ROCHEeim) in RPMI medium, then cut in small pieces and incubated at 37° C. for 15 min. To disrupt T-cell-DC complexes 4 μl of 0.5 M EDTA was added to the cell suspension and incubated at 37° C. for 5 min. After collagenase-D treatment a single cell suspension of splenocytes was prepared according to standard procedures. The cells (2×10⁶) were washed once with FACS buffer (5% FBS and 0.1% Na-azide in PBS) and nonspecific binding was blocked with rat anti-mouse CD16/CD32 Ab (clone 2.4 G2, BD PharMingen) for 10 minutes on ice. The cells were stained with the following rat anti-mouse monoclonal antibodies: allophycocyanin (APC) anti-CD11c (clone N418), PE Conjugated anti-NK 1.1 (clone PK136) from eBioscience and R-Phycoerythrin (R-PE)-conjugated anti-CD8α (clone 53-6.7), R-PE Conjugated anti-CD11b (clone M1/70), R-PE Conjugated anti-CD45R/B220 (clone RA3-6B2) from BD PharMingen and fixed in 2% paraformaldehyde in PBS. Cells (100,000 events per sample) were acquired on a FACSCalibur flow cytometer (Becton Dickson, San Jose, Calif.) and analyzed with FlowJo software (Treestar, San Carlos, Calif.). Cells were also visualized by fluorescence microscopy as described above.

Construction, propagation and purification of CPMV chimeras. The genome of CPMV consists of two single-strand positive-sense RNA molecules, both of which have been cloned in separate plasmids designated pCP1 and pCP2. Dessens and Lomonossoff. J. Gen. Virol. 74:889, 1993. The large and small subunit coat proteins are encoded in pCP2. Five different constructs were made, four with inserts in the βBβC loop of the small coat protein (between amino acids 22-23), and one with an insert in the βEαF loop of the large coat protein (between amino acids 98-99; Table 3). The oligonucleotide sequences were designed according to known CPMV codon usage. The modifications in the small coat protein were made using the vector pCP2-0.51 which contain the human rhinovirus (HRV) sequence in the βBβC loop. Dalsgaard et al., Nat. Biotechnol. 15:248, 1997. To construct pCP2-smGP33, the HRV sequence was removed from pCP2-0.51 by digestion with NheI and AatII and replaced by the oligonucleotides 5′-CT AGC ACT CCT CCT GCT AAG GCT GTG TAC AAC TTC GCT ACA TGT CCA TTT TCA GAC GT-3′ (the restriction sites NheI and AatII are underlined). Oligonucleotides were annealed by 3 cycles of heating (from 80° C. to 60° C.) before ligation. The plasmid pCP2-smGP33D contains an additional amino acid (D) at the 3′ end of the insert, which was added by site directed mutagenesis using a QuikChange™ Site-Directed Mutagenesis Kit from Stratagene. An extra amino acid (D) was added to keep the sequence of the insert intact after the natural cleavage of the βBβC loop in the plant, and to improve the yield of the virus chimera. The oligonucleotide sequences used for the plasmid constructs pCP2-smGP33-Myc and pCP2-smGP61D were: 5′-CT AGC ACT CCT CCT GCT AAG GCT GTT TAT AAT TTC GCA ACT ATG ACT AGT GAA CAA AAG TTG ATT AGT GAA GAA GAC TTG GGT CCA TTT TCA GAC GT-3′ and 5′-CT AGC ACT CCT CCT GCT GGA TTG AAA GGT CCT GAC ATC TAT AAA GGA GTC TAC CAA TTC AAG AGC GTC GAG TTC GAT GAT CCA TTT TCA GAC GT-3′, respectively. The modification in the large coat protein was made using the vector pLgEF, which contains HpaI and KpnI restriction sites in the βEαF loop. Chatterji et al., Intervirology 45:362, 2002. To make pCP2-lgGP33, the pLgEF vector was digested with the HpaI and KpnI restriction enzymes and ligated to the annealed oligonucleotide 5′-AGG GGT AAG GCT GTG TAT AAT TTT GCT ACT TGT AAGTAT AGT AC-3′. The amino acid sequences of the inserts are shown in Table 3.

TABLE 3
Amino acid sequence of the LCMV CTL epitopes inserted in CPMV external loops.

| | | | | | Yield |
|---|---|---|---|---|---|
| | | | | | (mg of |
| Insertion | CPMV | Amino acid | Mouse | MHC | virus/g |
| site | chimera | Sequences | strain | molecule | leaves) |
| βBβC | smGP33 | KAVYNFATC | C57BL/6 | class I (D^{b}) | 0.0 |
| loop^{(a)} | smGP33D | KAVYNFATCD | C57BL/6 | class I (D^{b}) | 0.35 |
| | smGP33Myc | KAVYNFATMTSEQKLISEEDLG | C57BL/6 | class I (D^{b}) | 0.36 |
| | smGP61D | GLKGPDYIKGVYQFKSVEFDD | C57BL/6 | class II (I-A^{b}) | 0.1 |
| βEαF | Lg33 | KAVYNFATC | C57BL/6 | class I (D^{b}) | 0.78 |
| loop^{(b)} | | | | | |

^{(a)}Insertions in the βBβC loop of the small subunit (between amino acids 22-23).
^{(b)}Insertions in the βEαF loop of the large subunit (between amino acids 98-99).

The pCP2 and pCP1 constructs were linearized with EcoRI and MluI restriction enzymes respectively. After enzyme inactivation, each pCP2 construct was individually mixed with pCP1 and co-inoculated onto 10 day-old cowpea plants as described by Dessens and Lomonossoff. Dessens and Lomonossoff. J. Gen. Virol. 74:889, 1993; Wellink, Plant Virol. 81: 205, 1998. From 10 to 15 days after inoculation, extracts from primary leaves were used as inoculums for passaging to new cowpea plants in order to produce virus-working stocks. All infected plants were characterized by RT-PCR and sequenced to verify the presence of the correct sequence of the foreign inserts in the recombinant CPMVs. The chimeric CPMVs were purified from the infected leaves by standard methods described by Wellink (44). The final virus pellets were resuspended in PBS (Gibco-BRL) and filtered sterilized through a 0.2 μm membrane (Costar). Virus concentration was measured by spectrophotometer using an E260=8, where absorbance at 260 is equal to 8 when virus concentration is 1 mg/ml at 1 cm light path.

SDS-PAGE, Western blotting and immunostaining. CPMV chimeras and wild-type CPMV were analyzed by electrophoresis using a linear gradient NuPAGE 4%-12% Bis-Tris pre-cast gels (Invitrogen, Carlsbad, Calif.) and transferred to Immobilon-P membranes (Millipore). Immunostaining for CPMV coat proteins was performed using a polyclonal anti-CPMV IgG purified from rabbit antisera on a protein G column (Amersham Pharmacia, Uppsala, Sweden). The immunostaining for the presence of the myc epitope was performed using a rabbit polyclonal anti-c-MYC antibody (Sigma). ImmunoPure Goat anti-Rabbit IgG, peroxidase conjugated (Pierce, Rockford, Ill.) was used as secondary antibody. Detection of peroxidase was carried out using SuperSignal West Pico Chemiluminescent Substrate (Pierce, Rockford, Ill.).

Detection of virus-specific CD8⁺ T-cell activity and ICCS assay. For the analysis of the primary virus-specific T cell response, spleens were harvested 12 days after lgGP33 immunization and for the secondary response 8 days after boosting. Negative controls were inoculated with CPMV-wt or PBS. For the positive control, mice were inoculated intraperitoneally with 2×10⁵ p.f.u. of LCMV 8 days before the assay. Single cell suspensions of splenocytes were prepared according to standard procedures and resuspended in RPMI 1640 medium supplemented with 10% FBS, 2 mM L-glutamine, 10 U/ml penicillin G, 100 μg/ml streptomycin, 1 mM Na Pyruvate, 0.1 mM non-essential amino acids, 10 mM HEPES (all from Gibco-BRL) and 5 mM β-mercaptoethanol (Sigma). Splenocytes (1×10⁶) were stimulated for 5 hours in 200 μl of RPMI complete medium with 1 μg/ml of GP33 peptide (PeptidoGenic, Livermore, Calif.) in the presence of 10-50 U/ml recombinant human IL-2 (Hoffmann-La Roche Inc., Nutley, N.J.) and 2 μg/ml Brefeldin A (Sigma). Staining of cell-surface antigen and intracellular antigens was performed as described by Homann. Homann et al., J. Virol. 72:9208, 1998. Briefly, the cells were first incubated for 10 min on ice with Fc blocking solution (anti-CD16/CD32, BD-PharMingen), and stained using PE-conjugated anti-CD8α (clone 53-6.7, BD PharMingen) for 30 min on ice and dark conditions. After washing, the cells were fixed and permeabilized in para-formahaldeyde (PFA)/saponin (Sigma) buffer (10 mM HEPES (Gibco), 4% PFA and 0.1% saponin in Hanks' Balanced Salt Solution (HBSS). For the intracellular staining, FITC-conjugated anti-TNF-α (clone MP6-XT22, BD-PharMingen) and APC conjugated anti-IFN-γ (clone XMG1.2, BD-PharMingen) in saponin buffer (0.1% saponin in FACS buffer) were used. Stained cells were acquired on a FACSort flow cytometer (100,000 events per sample) and analyzed with Cell Quest (Becton Dickinson) and FlowJo (Tree Star, Inc.) software.

CPMV immunization and LCMV challenge. Mice were inoculated i.p. with 200 μg CPMV-wt or CPMV chimeras. At 10 weeks post-immunization, mice were boosted i.p. with the same amount of CPMV chimeras. Negative control mice were inoculated with PBS and positive control mice were immunized with a single i.p. injection of 2×10⁵ PFU of LCMV. For the LCMV challenge assay groups of 8 or 6 mice were inoculated i.p. with 200 μg of CPMV chimeras, 200 μg of CPMV-wt (negative control), PBS (negative control) and 2×10⁵ PFU of LCMV (positive control). At 6 weeks post-inoculation mice were challenged by intracranial (i.c.) injection with 30 LD₅₀ units of LCMV. Mice were monitored for a minimum of 20 days following virus challenge for morbidity and mortality.

### Example 20

Antigen Presenting Cells Bind and Internalize CPMV Particles In Vitro

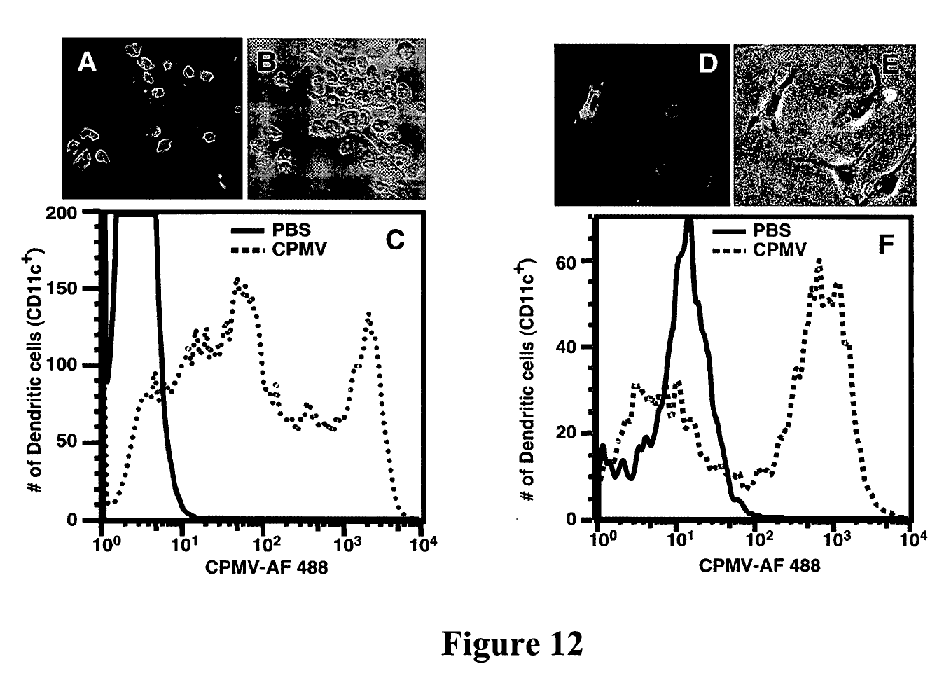

The use of CPMV nanoparticles to induce T cell responses in vivo requires their interaction with APCs. the ability of APCs to bind and internalize the CPMV plant virus was analyzed. In order to visualize the virus by fluorescent microscopy and detect it by FACS, the virus particles were labeled on external cysteines or lysines using Fluorescein (F)-maleimide or NHS-Alexa Fluor 488 (AF488) (see Materials and Methods), obtaining 23 and 71.33 dyes per virus particle respectively. Next the binding of CPMV to primary murine bone-marrow derived DCs, and to the Balb CL7 and MC57 cell lines was studied. Fixed cells were incubated overnight on ice with fluorescent-labeled CPMV and visualized using a fluorescence microscope. The binding of CPMV to cells from 10 day-differentiated bone marrow culture is shown in FIGS. 12A and 12B. CPMV particles were capable of binding to cell membranes, which is demonstrated by a green appearance on the surface of some cells. In this culture, only 40% of the cells were CD11c⁺, suggesting that the cells that were negative for CPMV binding were likely not DCs. Next, the binding of CPMV to DC positive cells was quantitatively determined by FACS using the CD11c and B7.1 antibody markers. Bone marrow cultures containing 62% of CD11c⁺ cells were fixed and incubated with CPMV overnight on ice. After staining with CD markers, the cells were analyzed by FACS (FIG. 12C). The results indicated that 83% of the CD11c⁺ DC bound CPMV particles. Two populations of DCs were identified: one group with high CPMV binding and a second group with low binding. FACS analysis using the B7.1 antibody marker gave similar results. Murine Balb CL7 and MC 57 cell lines also bound CPMV particles. These cells were fixed and incubated with virus particles overnight at 4° C. and visualized by the fluorescent microscope. All cells were fluorescent on their surface, suggesting that the CPMV particles are able to bind to their membranes.

FIG. 12 shows binding and uptake of CPMV particles by bone marrow dendritic cells. (A, B and C) CPMV binding: DCs were fixed with 2% formaldehyde and incubated with CPMV-AF488 on ice overnight. The cells were stained with Hoechst 33258 to visualize the nucleus or stained with the APC-CD11c antibody marker to quantify DCs. (A) Cells were visualized by immunofluorescent microscope (20× objective). CPMV particles are in green and the nucleus in blue. (B) Transmission light image showing the body of the cells. (C) FACS analysis showing the binding of CPMV particles to CD11c⁺ dendritic cells. (D, E and F) Uptake of CPMV by DCs: DCs were incubated for two hours with CPMV-AF488 at 37° C. and 5% CO₂. (D) Cells were fixed with 2% formaldehyde stained with Hoechst 33258 and visualized with the immunofluorescent microscope using a 20× objective. CPMV particles are in green and the nucleus in blue. (E) Transmission light image showing the body of the cells. (F) After 2 hours incubation with CPMV the cells were stained with the DC marker CD11c and analyzed by FACS to quantify the internalization of CPMV particles into CD11c⁺ DCs. PBS was added to the control cells.

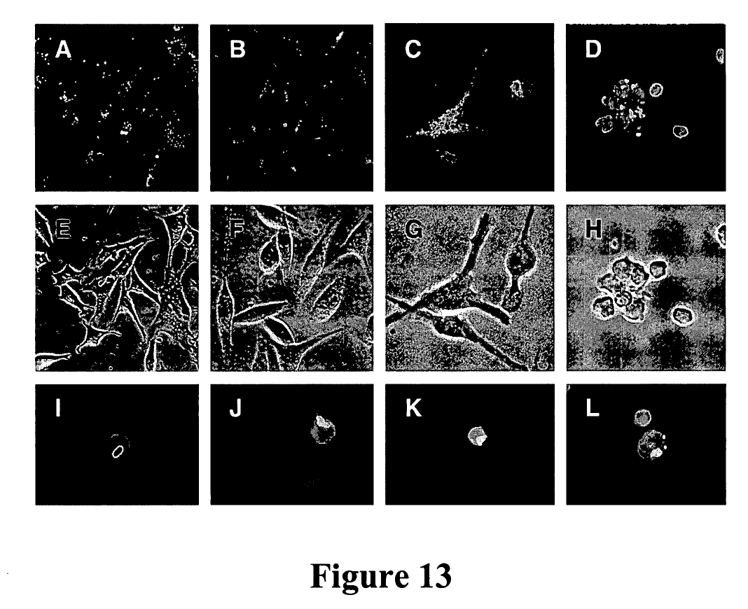

To evaluate the ability of antigen presenting cells (APC) to internalize CPMV particles, the in vitro uptake of CPMV by Balb C17 and MC57 fibroblast was first analyzed by fluorescent microscopy. The cells were cultured overnight with CPMV-F, and then stained with DAPI to visualize the nucleus. As shown in FIGS. 13A and 13B, the cells contain green fluorescent vesicle-shape structures, indicating the ability of Balb C17 and MC 57 cells to internalize CPMV particles. The uptake of CPMV particles by DCs in vitro was also investigated. The source of DCs was bone marrow cells from C57 BL/6 mice. Differentiated bone marrow cells were allowed to adhere to the surface of 6 well plates overnight. The cells were then incubated with CPMV AF488 for two hours or overnight. After staining the nucleus with Hoechst, the cells were visualized under the fluorescent microscope. FIGS. 12D and 13C show the presence of CPMV green fluorescent structures inside the cells after two hours or overnight incubation, respectively.

To quantify the internalization of CPMV by DCs, a culture of 9 day-differentiated bone marrow cells were incubated for two hours with CPMV AF488, then stained with DC markers and analyzed by FACS. The following DC antibody markers were used: anti-CD11c, anti-B7.1, and anti-B7.2. As shown in FIG. 12F, 60% of CD11c⁺ cells were positive for CPMV while 40% did not internalize the fluorescent virus particles. Cells stained with B7.1 or B7.2 uptake the CPMV particles with 88% and 70% of cells internalizing the virus, respectively. These results indicate that CD11c+, B7.1+ and B7.2+DCs are able to efficiently internalize CPMV particles in vitro. The binding and internalization studies both support the finding that a subpopulation of DC are not competent to take up CPMV.

### Example 21

Antigen Presenting Cells Internalize CPMV Particles In Vivo

To determinate whether CPMV internalization by APCs occurs in vivo, C57 BL/6 mice were inoculated i.p. using 100 μg of CPMV labeled with AF488. Four hours later, the spleens were removed and a single cell suspension of spleen was prepared after collagenase treatment. After fixing and staining the nucleus with Hoechst, the cells were visualized under the fluorescent microscope. FIG. 13D shows spleen cells containing green fluorescent vesicles, indicating CPMV uptake. To identify which kind of spleen cells internalize the virus particles, the surface of the cells were stained with the following antibody markers: PE-CD11c (DC), PE-CD8α, PE-B220 (B-cells) and PE-NK 1.1 (natural killer cells), and visualized under the microscope. As shown in FIGS. 13I, 13J, 13K and 13L, CD11c cells, B cells, CD8α cells and natural killer cells were able to internalize CPMV.

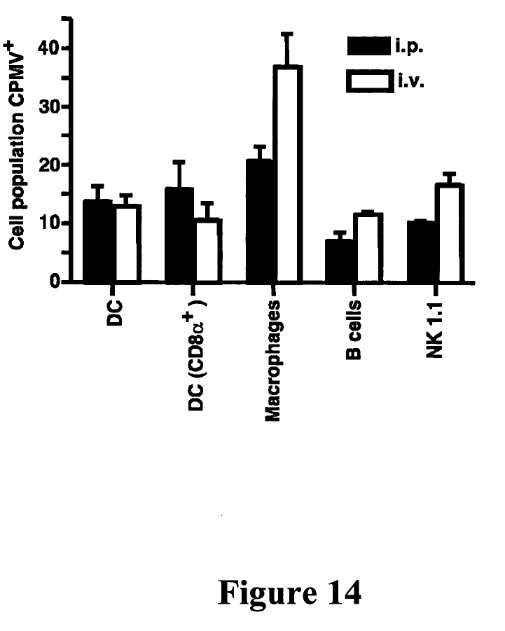

Based on these findings, the in vivo uptake of CPMV particles by different populations of cells was evaluated quantitatively. It was also studied whether the route of inoculation could affect the amount of cells able to internalize CPMV. Groups of 3 mice were inoculated i.v. or i.p. with 100 μg of CPMV-AF488 (1×10¹³ virus particles) and 4 hours later the spleens were removed and the cells analyzed by FACS. The following populations of cells were studied: DC (CD11c+), a subpopulation of DC specialized in cross-presentation (CD11c⁺, CD8α⁺), macrophages (CD11c⁻, CD11b⁺), B cells (B220⁺), and natural killer cells (NK 1.1⁺). As shown in FIG. 14 all cells were able to internalize CPMV particles, corroborating the results observed under the fluorescent microscope. FIG. 14 also shows that the i.p. route generated 5% more DC (CD8α+) CPMV positive cells than the i.v. route, which is important for exogenous antigen presentation through the MHC class I molecules. In contrast the i.v. route of inoculation resulted in a higher percentage of macrophages, B cells and NK cells capturing CPMV particles than the i.p. route.

FIG. 13 shows binding and internalization of CPMV nanoparticles in vitro and in vivo. (A and B) In vitro uptake of CPMV-F by the cell lines Balb C17 and MC 57, respectively. (C) In vitro uptake of CPMV-AF488 by DCs obtained from C56 BL/6 bone marrow cells. Balb C17, MC 57 and DCs were incubated overnight with the fluorescent virus particles (in green), washed with PBS, fixed, and stained with DAPI (A, B) or Hoechst 33258 (C) to visualize the nucleus. (D and I-L) In vivo uptake of CPMV-AF 488. (D) Spleen cells were purified 4 hours after i.p. inoculation with 100 μg CPMV-Alexa. Following purification, cells were stained with: (I) PE anti-CD8α (red), (J) PE anti-CD11c (red), (K) PE anti-B220 (red), and (L) PE anti NK 1.1 (red). E, F, G and H are the transmission light images showing the body of the cells corresponding to A, B, C and D pictures. The virus particles are in green and the nucleuses are in blue.

FIG. 14 shows uptake of CPMV in vivo. Groups of three C57BL/6 mice were inoculated i.p. (black columns) or i.v. (white columns) with 100 μg of CPMV-AF488. After 4 hours, spleens were removed and the cells stained to analyze the internalization of the CPMV into CD11c-positive DCs, CD11c/CD8α-positive DCs, macrophages, B-lymphocytes, and NK cells. After staining, the cells were fixed and analyzed by FACS. Mice inoculated with PBS were used to subtract the background.

### Example 22

Display of T-Cell Epitopes on CPMV

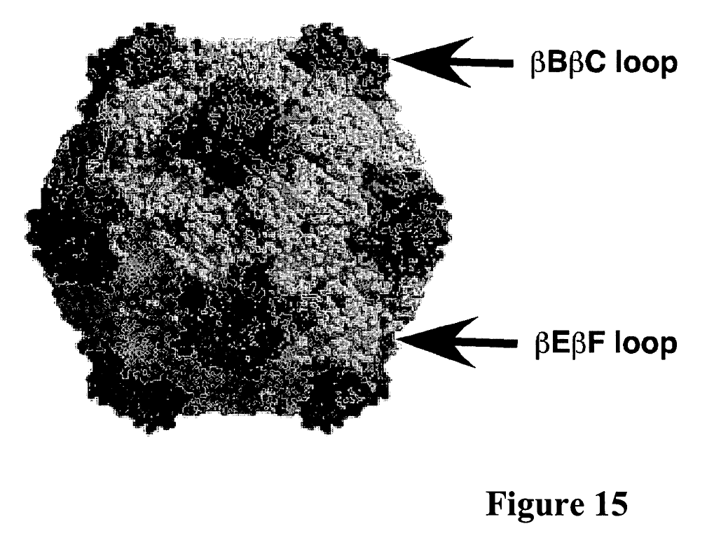

The above results indicated that CPMV particles are efficiently captured by professional APCs in vivo, including the CD8α⁺ subset of DCs that specialize in presenting peptides from exogenous antigens to CTLs. To use CPMV as a carrier of CTL peptides the genome of the virus was modified by inserting the sequence of the GP33 peptide (CTL epitope) or the GP61 peptide (T helper epitope) from LCMV into the coat proteins of CPMV. FIG. 15 shows a model of the structure of CPMV particles. The peptides were inserted into two different solvent-exposed sites: the βBβC loop (red) in the small coat protein (dark grey), and the βEαF loop (violet) of the large coat protein (light grey). Three different versions of the GP33 peptide were inserted in the small coat protein (Table 3). The plants did not show any signs of infection after inoculation with the recombinant plasmid containing the GP33 sequence KAVYNFATC, indicating that this sequence interferes with the formation of the chimeric virus particles smGP33 (sm for the small coat protein). After modifying the sequence by adding an aspartic acid at the C-terminal of the GP33 peptide, the virus chimera (smGP33D) was able to assemble properly and produced lesions in plants similar to wild-type CPMV. This result is consistent with that of Porta et al., who showed that the isoelectric point of insertions displayed on CPMV influences the yield of particles. Porta et al., Virology 310:50, 2003. To detect the presence of the foreign peptide in the capsid of the virus by western blot, the c-myc epitope (EQKLISEEDL) was introduced at the C-terminal side of the GP33 peptide (smGP33Myc chimera). The smGP33Myc chimera gave wild-type lesions in the cowpea plants and the yield was similar to the smGP33D chimera, obtaining 0.35-0.36 mg of chimeric virus per gram of leaves (Table 3). The T helper epitope GP61 was also inserted in the small subunit (smGP61D chimera), giving a lower yield 0.1 mg of chimeric virus per gram of leaves.

To investigate whether the location of the CTL epitope on the coat protein of the CPMV influences the CTL immune response, the GP33 peptide was also introduced in the βEαF loop of the large coat protein. The resulting chimera was named lgGP33 (1 g for large coat protein). This chimera replicated and propagated very efficiently in the cowpea plants giving a yield of 0.78 mg of chimeric virus per gram of leaves, which is close to the CPMV wild type yield (1 mg virus/g leaves).

FIG. 15 shows a model of the CPMV structure showing the small coat protein pentamers in dark grey and the large coat pentamers in light grey. The peptides were inserted into two different solvent-exposed sites: a) the βBβC loop (red) in the small coat protein, and b) the βEαF loop (violet) of the large coat protein. Wang et al., Chem. Biol. 9:813, 2002.

### Example 23

Analysis and Characterization of CPMV Chimeras

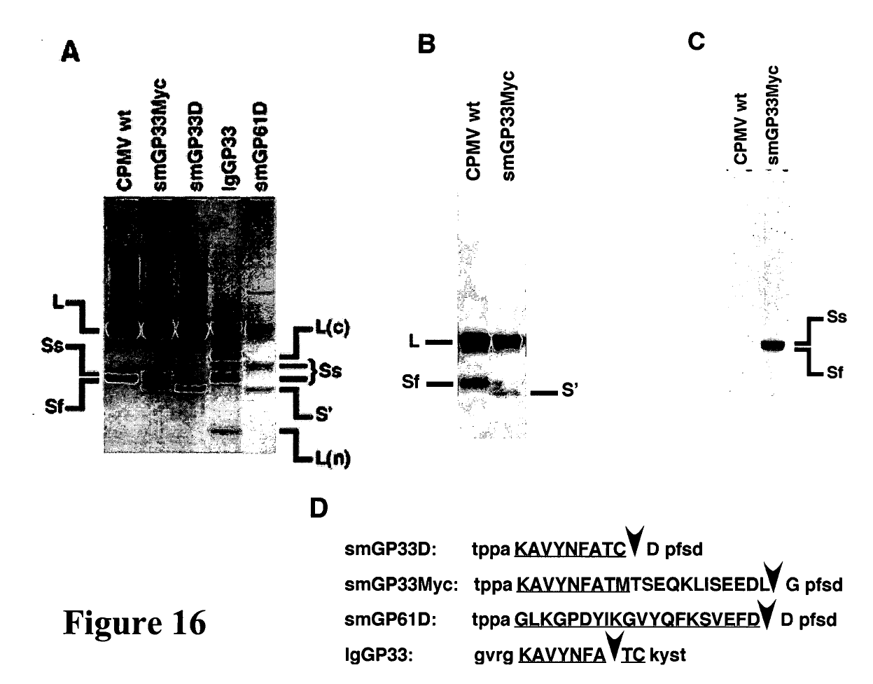

The chimeric viruses were examined by electrophoresis on SDS-PAGE and western blotting using IgG anti-CPMV and anti-c-myc polyclonal antibodies (FIG. 16). The SDS-PAGE on FIG. 16A shows two bands for wild-type CPMV, the higher corresponding to the large coat protein (L) with a molecular weight of 40 kD, and the lower band to the small coat protein fast component (Sf) of 24 kD. The Sf band represents the processed form of the S coat protein. The wild type S, as well as the chimeric S coat proteins undergo a proteolytic cleavage which removes the 24 C-terminal amino acids. This proteolytic event occurs either in the plants or during purification and generates two electrophoretic forms, the fast form (Sf) and the slow form (Ss). The chimera smGP33Myc contains three bands for the small coat protein: a) the slower migrating band (Ss), which is larger than the wt-S protein and corresponds to the unprocessed small coat protein including the foreign insert, b) the middle band (Sf) which corresponds to the processed S protein with a slightly higher MW than the wild type protein due to the insert, and c) the fastest migrating band (S′) representing the processed S protein which undergoes a proteolytic cleavage between the two C-terminal amino acid residues of the insert (FIG. 16D). The peptide (minus the last amino acid) remains fused to the 22 residues of the N-terminus, this small fragment is not retained on the SDS-PAGE gel due to its small size, but in the native particle it remains associated to the coat protein by non-covalent forces. Lin et al., Fold. Des. 1:179, 1996. Because this chimera has an additional amino acid (D) at the C-terminal of the GP33 peptide, the cleavage event does not disrupt the sequence of the CTL epitope. Thus, Sf and Ss, but not S′, contain the insert as show in the western blot on FIG. 16C. These bands reacted with the c-myc-specific antibody by western blot, indicating that the chimeric virus had both the c-myc and the GP33 epitopes. The smGP33D chimera showed 2 bands on the SDS-PAGE gel: the L and only the S′ for the small coat protein. Thus, the S coat protein from the smGP33D chimera undergoes 100% cleavage and the S protein from smGP61D is 50% cleaved (FIGS. 16A, 16D).

The chimera lgGP33 shows 3 protein bands for the large coat protein (FIG. 16A). The largest polypeptide corresponds to the full size L protein plus the insert and migrated slightly slower that the wt-L protein. The other two L bands correspond to the polypeptide products of a proteolytic cleavage event in the GP33 insert (FIG. 16D), generating the C-terminal L(c) and the N-terminal L(n) fragments of the large coat protein. To investigate if the cleavage event was occurring inside the GP33 epitope, protein sequencing of the amino-terminal fragment of the L(c) polypeptide was performed by mass spectrometry. This result indicates that cleavage of CPMV chimeras can also occur within insertions in the βEαF loop as has been seen by Brennan et al. Brennan et al., Microbiology 145:211, 1999. Cleavage occurs between the amino acids alanine and threonine of the GP33 peptide, disrupting the sequence of the epitope (FIG. 16D). This proteolytic event occurs with a 50% frequency, thus the lgGP33 chimera has an average of 30 copies of the intact GP33 epitope per particle and the remaining chimeras have a total of 60 copies of the peptide/particle available for presentation by APCs.

FIG. 16 shows protein analysis of the purified CPMV chimeric virions. (A) Simple blue-stained 4-12% polyacrilamide-SDS gel showing the coat proteins from CPMV wild type and the chimeras: smGP33-Myc, smGP33D, lgGP33 and smGP61D. (B) Western blot detecting CPMV proteins using an anti-CPMV specific IgG polyclonal rabbit antiserum: CPMV wild type and smGP33-Myc chimera. (C) The transfer membrane from B was washed and used again to detect the myc epitope using a rabbit polyclonal anti-c-MYC antibody. Bands corresponding with proteins are indicated: Ss, small coat protein slow form; Sf, small coat protein fast form; S′, small coat protein cleavage product lacking the N-terminal 23 amino acids and insert; L, large coat protein; L(n), N-terminal cleavage product of the large protein; and L (c), C-terminal cleavage product of the large protein. (D) Cleavage site and amino acid sequences of the peptides inserted in the βBβC loop (chimeras: smGP33-Myc, smGP33D and smGP61D) and in the βEαF loop (lgGP33 chimera). The arrow indicates the cleavage site, the c-myc epitope is in bold, the GP33 and GP61 epitopes are in bold and underlined, the extra amino acids are in grey and four of the CPMV flanking amino acid are in lowercase.

### Example 24

CPMV lgGP33 Chimera Induces Antigen-Specific T Cell Cytokine Expression

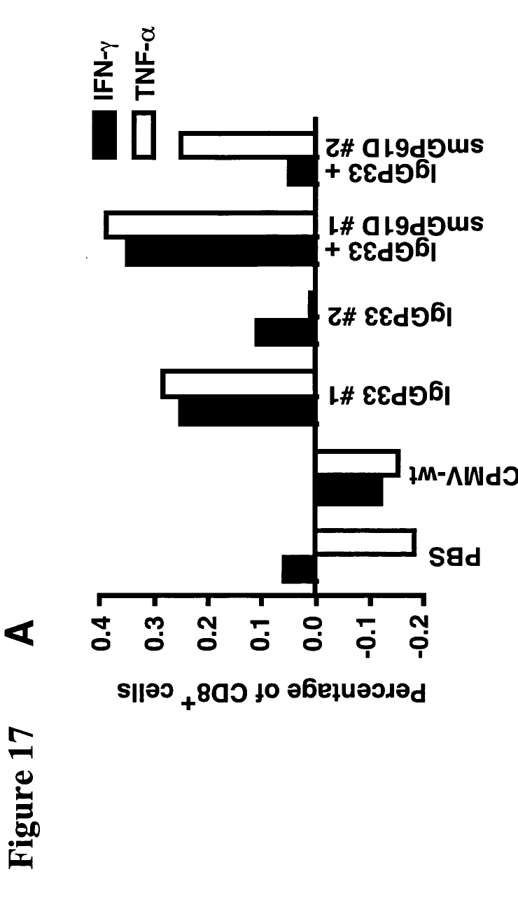

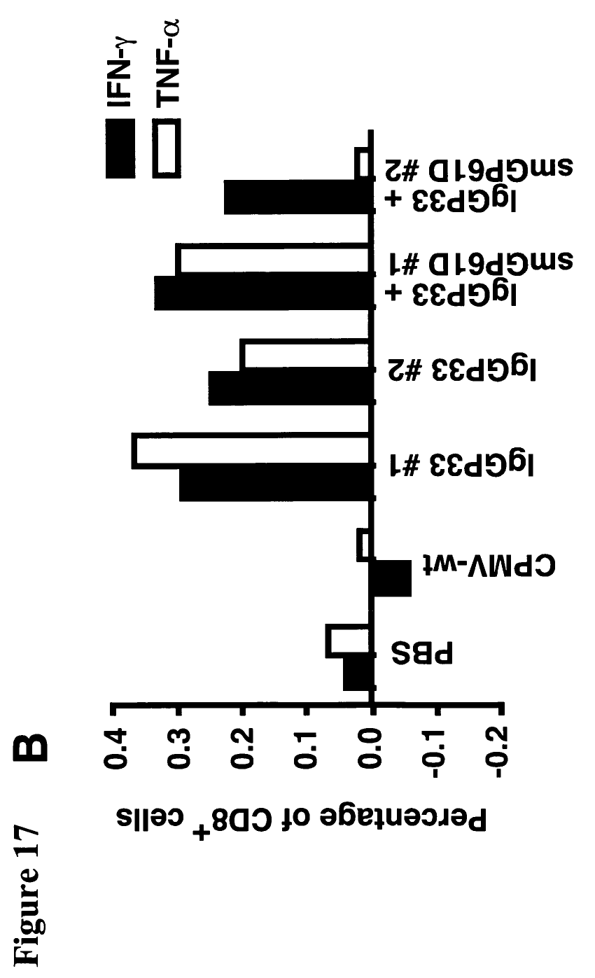

To study the capacity of the CPMV chimeras to induce cytokine production in CD8⁺ T cells, C57BL6/J mice were immunized i.p. with 200 μg of the lgGP33 chimera (FIG. 17A). The lgGP33 and the smGP61D chimera was also mixed to study the possible contribution of the T helper epitope after a second inoculation (FIG. 17B). Twelve days after, a direct ex-vivo analysis was performed to measure the percentage of CD8⁺ T cells producing IFN-γ and TNF-α cytokines by the ICCS assay. As shown in FIG. 17A, an increase was detected in the percentage of peptide-specific CD8⁺ T cells producing IFN-γ in two out of 4 mice and an increase on TNF-α production in 3 out of 4 mice. The infection with LCMV as a positive control gave 10% of CD8⁺ T-cells producing IFN-γ and 6.5% producing TNF-α. The average results of peptide-specific response for smGP33Myc chimera was approximately 0.23% for IFN-γ and 0.42% for TNF-α over the CPMV-wt control and for the smGP33D were 0.68% for IFN-γ and 1.74% for TNF-α over the CPMV-wt.

The IFN-γ cytokine values obtained from the group of mice analyzed 8 days after boosting were more consistent than the group of mice that only received a primary inoculation. However, the T-helper epitope did not contribute to an increase in the percentage of CD8⁺ T cell producing cytokines, as the values of the primary and secondary immune responses were similar (FIG. 17B). In this assay the LCMV positive control responses were 13.53% for IFN-γ and 7.3% for TNF-α. Moreover, these results also show that the CD8⁺ T cells primed by CPMV vaccines exhibit different cytokine expression profiles than the responses induced by the replicating antigen LCMV. For LCMV the IFN-γ response is typically 1.53 fold higher than the TNF-α response, whereas for the lgGP33 CPMV response (FIG. 17B) the IFN-γ:TNF-α ratio averages are approximately 1.22.

### Example 25

Protection from Lethal Viral Challenge

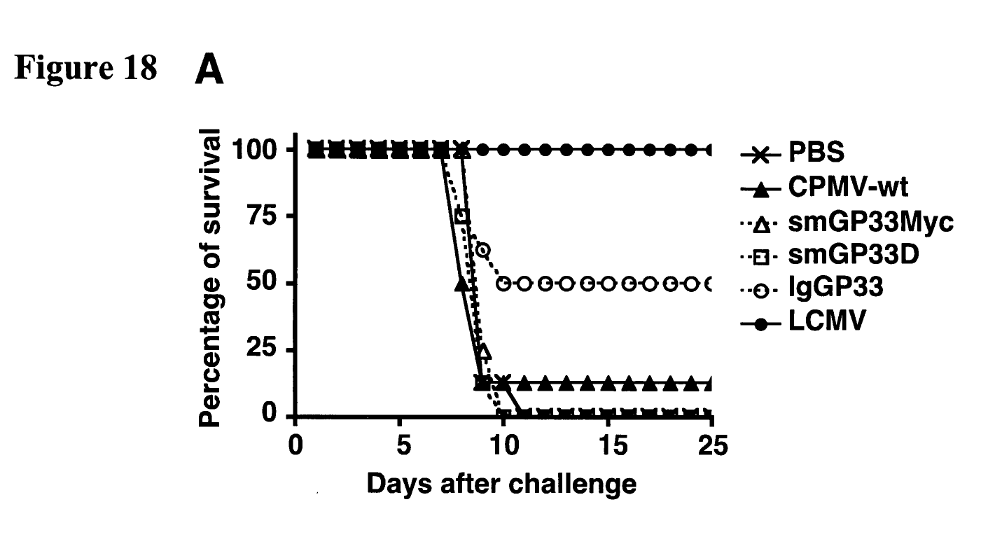

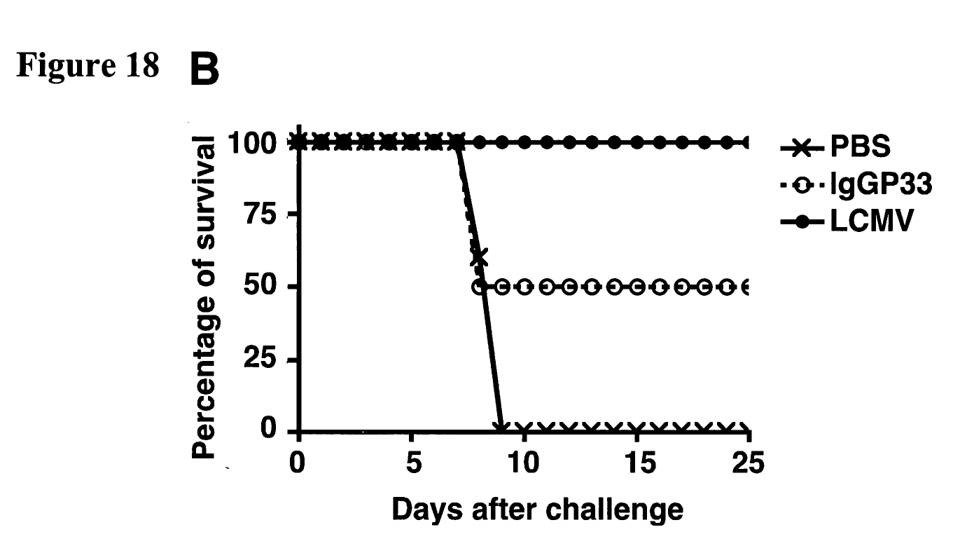

It was next determined whether the number of cytokine producing cells observed after the administration of CPMV vaccines were sufficient to protect mice against a LCMV lethal challenge. Groups of 6 mice were vaccinated i.p. with a single dose (200 μg) of the smGP33D, smGP33Myc or lgGP33 chimeras. Twelve days after the inoculation, the mice received an intracranial (i.c.) injection of LCMV Armstrong (30 LD₅₀ units). As a positive control, one group of mice was inoculated with 2×10⁵ p.f.u. of LCMV 6 weeks before the challenge. The negative control group of mice was inoculated with PBS or wt-CPMV. As shown in FIG. 18A, immunization with LCMV (positive control) 6 weeks prior to challenge resulted in the acquisition of immunity, which enabled the mice to survive the lethal i.c. inoculation. In addition, all mice sham-immunized with PBS died upon challenge as was expected. Fifty percent of the mice vaccinated with the lgGP33 chimera were protected and survived the LCMV challenge. This result correlates with the ICCS ex-vivo experiment (FIG. 17A) where 2 out of 4 mice show an increase in the percentage of cytokine-producing CD8⁺ T cells. In contrast, none of the mice vaccinated with the smGP33D or smGP33Myc chimeras survived beyond 10 days post challenge. The difference between these chimeras and the lgGP33 is the location on CPMV where the peptide was inserted. Surprisingly, one of six mice immunized with wt-CPMV survived the lethal challenge.

To determine if a second dose of lgGP33 chimera would increase the level of protection against lethal LCMV challenge, a group of 6 mice vaccinated with lgGP33 were boosted 4 weeks after the first inoculation. Mice inoculated intraperitoneally with PBS or with LCMV (2×10⁵ p.f.u.) were the negative and positive controls respectively. Eight days after boosting, mice were challenged i.c. with a lethal dose of LCMV (30 LD₅₀). All mice inoculated with LCMV 6 weeks before survived and all mice previously inoculated with PBS died between 8 and 9 days post challenge. The lgGP33 chimera again provided protection to 50% of the mice against lethal challenge, as was previously seen following the primary immunization.

FIG. 17 shows cytokine expression following CPMV chimera immunization. LCMV-specific CD8⁺T cells producing IFN-γ and TNF-α are detectable directly ex vivo 12 days after primary immunization or eight days after boosting. C57BL/6 mice were immunized intraperitoneally with: lgGP33 (200 μg), lgGP33 mixed with smGP61D (200 μg each), and CPMV-wt (200 μg) and PBS as negative controls. (A) Twelve days later, splenocytes were assayed directly ex vivo for detection of CD8α cells expressing IFN-γ and TNF-α. After 5 hours of (GP₃₃₋₄₁) peptide stimulation in presence of brefelding A and Il-2, CD8α cells and intracellular cytokines were stained and analyzed by flow cytometry as indicated in materials and methods. (B) Ten weeks after primary immunization, mice were boosted and the vaccinations were performed as indicated above. Eight days after boosting, splenocytes were assayed and CD8α cells producing cytokines analyzed by ICCS and flow cytometry as in (A).

FIG. 18 shows CPMV chimera protection from a lethal virus challenge. (A) Groups of 8 C57BL/6 mice were primed by i.p. immunization with 200 μg of CPMV chimeras: lgGP33, smGP33D, smGP33myc or CPMV-wt. PBS inoculated mice were used as negative controls and LCMV i.p. infected mice (2×10⁵ p.f.u.) as positive controls. Six weeks later, mice were challenge intracerebrally with 30 p.f.u. of LCMV ARM (30 LD₅₀). (B) Groups of six C57BL/6 mice were primed by i.p. immunization with 200 μg of lgGP33, PBS or 2×10⁵ p.f.u. of LCMV. Six weeks later, lgGP33 vaccinated mice were boosted with 200 μg of the same chimera and the PBS group with PBS. Eight days after boosting, mice were challenged i.c. with 30 p.f.u. of LCMV ARM (30 LD₅₀).

### Example 26

Cellular Immune Response to a Chimeric Plant Virus Vaccine In Vivo

The present study demonstrates for the first time that a chimeric plant virus vaccine can generate cellular immune responses in vivo. A CPMV chimera displaying a single viral CTL epitope, the GP33 epitope from LCMV, was able to protect 50% of mice challenged intracranially with a lethal dose of LCMV. Protection correlated with the induction of GP33-specific MHC class I-restricted CD8⁺ T cells, as demonstrated by direct ex-vivo CTL assay. It was also shown that antigen-presenting cells are able to internalize CPMV particles both in vitro and in vivo. In vivo, CPMV particles are found in DCs (both CD8α⁺ and CD8α⁻), macrophages, B cells and natural killer cells following i.p. or i.v. inoculation. In the immune system, DCs and macrophages specialize in processing exogenous antigens that prime the CTL response. den Haan et al., J. Exp. Med. 192:1685, 2000; Huang et al., Immunity 4:349, 1996; Bohm et al., J Immunol. 155:3313, 1995; Huang et al., Science 264:961, 1994; Albert et al., Nature 392:86, 1998; Bellone et al., J Immunol. 159:5391, 1997.

Although CPMV particles contain an intact viral RNA genome, they function as virus-like-particles (VLPs) in mammalian systems since they have not been shown to replicate or express proteins in mammalian cells. Therefore, the peptide epitopes derived from CPMV and presented in an MHC class I-restricted fashion are exogenous antigens, relying on the ability of the DCs or macrophages to channel peptides from exogenous proteins into the MHC class I presentation pathway and undergo cross-presentation. VLPs are devoid of any viral genome and like CPMV no endogenous protein expression takes place in the host. Several VLP systems have been developed to induce CTL responses, such as hepatitis B virus-, parvovirus-, bacteriophage Qβ-, and papillomavirus-like particles. Sedlik et al., Proc. Natl. Acad. Sci USA 94:7503, 1997; Moron et al., J. Exp. Med. 195:1233, 2002; Storni et al., J. Immunol. 168:2880, 2002; Liu et al., Virology 273:374, 2000; Liu et al., Immunol. Cell Biol. 80:21, 2002; Storni et al., J. Immunol. 172:1777, 2004; Bachmann et al., J. Immunol. 173:2217, 2004. While several of these systems have been used to induce LCMV specific responses, the values from these data may be difficult to compare due to the different assay conditions and methods used by the investigators. However, the average responses obtained using VLPs in the absence of adjuvant by ELISPOT assay are in the range of 0.0065% for HPV-VLP to 0.03% for PPV-NP118-VLP. Da Silva et al., Vaccine 21:3219, 2003; Sedlik et al., J Virol 74:5769, 2000. Although the VLPs HBcAg-GP33 and bacteriophage Qβ-GP33 do not generate detectable numbers of effector T cells by ELISPOT, the tetramer staining assay gives responses in the range of 0.1% over the control for HBcAg-GP33 VLPs to 2.3% for bacteriophage Qβ-GP33 VLP. Storni et al., J. Immunol. 172:1777, 2004; Bachmann et al., J. Immunol. 173:2217, 2004. Moreover, tetramer staining measured 0.6% of specific CD8⁺ T cells for HPV-VLP from total splenocytes. Da Silva et al., Vaccine 21:3219, 2003. However, none of the VLP-induced T-cell responses using the GP33 epitope have been shown to protect animals against LCMV challenge. A porcine parvovirus (PPV) VLP displaying the NP118 CTL epitope from LCMV has been developed, and this PPV-NP118 VLP fully protects BALB/6 mice against virus challenge in the absence of adjuvant or co-stimulatory molecules. Sedlik et al., Proc. Natl. Acad. Sci USA 94:7503, 1997. In Balb/c mice the NP118 epitope comprises 90% of the T-cell repertoire against LCMV, whereas in C57Bl/6 mice the GP33 epitope accounts for approximately 30% of the virus-specific response. In the CPMV display system the lgGP33 chimera protects 50% of C57BL/6 mice from lethal challenge, indicating that CPMV is also a promising vaccine system. However, to be able to fully compare the CPMV and PPV vaccine platforms it will be important to construct CPMV chimeras displaying the LCMV NP118 epitope and compare its ability to protect BALB/6 mice against viral challenge.

Surprisingly, the protection of the CPMV chimeras against virus challenge was found only when the GP33 peptide was located in the βEαF loop of the large coat protein, (the lgGP33 chimera). However, the results from the direct ex-vivo CTL assay were similar in mice inoculated with the CPMV chimeras displaying the GP33 epitope in the small or in the large coat protein. These results suggest a difference in the quality of the cellular immune response between these virus chimeras. One explanation may be differences in the ratio of IFN-γ to TNF-α producing cells; for example, for LCMV and lgGP33 the IFN-γ:TNF-α ratio averaged 1.53 and 1.03 respectively, while for smGP33D and smGP33Myc the IFN-γ:TNF-α ratio averaged 0.48 and 0.27 respectively. The increased TNF-α response seen with these chimeras may not result in the efficient induction of CTL or memory cells. More subtle differences in T-cell responses might be observed when studying reductions in splenic LCMV titers following challenge. In addition, in some cases flanking sequences have been shown to be important for the efficient processing of epitopes for presentation by MHC class I molecules. Del Val et al., Cell 66:1145, 1991; Rueda et al., J. Gen. Virol. 85:563, 2004. It is possible that the neighboring residues in the βBβC loop of the small coat protein affect the processing of the antigenic sequence inducing a slightly weaker immune response and a failure of the smGP33D or smGP33-myc chimeras to protect mice against viral challenge. Nevertheless, although the response against GP33 using the lgGP33 chimera (approximately 0.035% CD8⁺ cells producing IFN-γ out of the total splenocyte population) is approximately 10-fold lower than the reported threshold level needed to observe protection from intracranial challenge, 50% protection is still observed. Berger et al., Virology 266:257, 2000.

Interestingly, priming with the CPMV-lgGP33 chimera generates GP33-specific CD8⁺ T cells and protects mice against viral challenge after primary immunization, but fails to increase the percentage of GP33-specific CTL cells or increase protection after boosting, where protection is still 50%. These results suggest that APC activation during CTL priming using CPMV-lgGP33 may not be sufficient to induce development of memory CTLs, or that the IFNγ:TNFα ratio may not be optimal for the induction of memory. When the mice were inoculated with the CPMV-lgGP33 together with the CPMV-smGP61D chimera containing the LCMV T helper epitope GP61, the CTL response also did not increase after re-exposure with the antigen, indicating that the T helper epitope is not being presented by APCs. It is possible that the site of the insertion for the GP61 epitope is not optimal to be processed correctly by the cells. Alternatively, efficient CD4⁺ stimulation using CPMV may require display of both the helper and CTL epitope on the same particle. The induction of Ag-specific cellular immune responses in vivo is critically dependent on several factors: the presentation of Ag-specific peptides, the expression of costimulatory molecules by the APC such as the CD40 ligand, and the cytokine environment during the interaction of APCs with specific T cells. To facilitate the induction of protective immunity and increase the lgGP33 chimera protection up to 100% it may be required to apply the chimeric particles together with factors that trigger APC activation such as anti-CD40 Abs or DNA rich in CpG motifs. It has been shown previously that Hepatitis B VLP carrying the GP33 peptide from LCMV failed to induce a response that protected mice from viral challenge in the absence of adjuvant. Storni et al., J. Immunol. 168:2880, 2002. However, the Hepatitis B GP33 VLPs injected together with agonistic anti-CD40 Abs or with CpGs are able to fully immunize against challenge with LCMV. Storni et al., J. Immunol. 168:2880, 2002; Bachmann et al., J. Immunol. 173:2217, 2004. Bachmann et al. also demonstrated that Hepatitis B GP33 VLPs encapsulating CpGs efficiently triggered the expansion of memory CTLs after a secondary challenge and this response correlated with increased frequencies of IFNγ producing GP33-specific CD8⁺ T cells. Bachmann et al., J. Immunol. 173:2217, 2004. For CPMV-lgGP33 chimeras the 50% protection observed in the absence of CpGs or other adjuvants may be due to the presence of plant viral RNA inside the capsid, which is not found in VLPs and could provide a stimulatory effect similar to viral ssRNA in APCs. Diebold et al., Science 303:1529, 2004; Heil et al., Science 303:1526, 2004. Finally, it is possible that the humoral immune response against the CPMV coat protein inhibits the secondary response against the GP33 epitope. However, Ruedl et al. and Sedlik et al. have shown that anti-VLP antibodies do not inhibit responses against CTL epitopes presented on VLPs. Ruedl et al., J Virol 79:717, 2005; Sedlik et al., J Virol 73:2739, 1999.

The differences in the level of protection comparing the CPMV-lgGP33 chimera, Hepatitis B GP33-VLP and PPV np118-VLP could also be related to the processing pathway used by these antigens. There are several mechanisms responsible for the processing of exogenous antigens in the MHC class I pathway: a) TAP- and proteaosome-independent and b) TAP- and proteasome-dependent pathways, where the latter has been shown to be more efficient than the former. The first one is based on the regurgitation of antigens or on the recycling of MHC class I molecules, in which the antigens are degraded in endosomes where they bind to the MHC class I molecules. Kovacsovics-Bankowski and Rock, Science 267:243, 1995; Rodriguez et al., Nat. Cell Biol. 1:362, 1999; Gromme et al., Proc Natl Acad Sci USA 96:10326, 1999; Chefalo and Harding. J. Immunol. 167:1274, 2001. The second mechanism involves the transfer of antigens from endosome/phagosome to the cytosol, protein processing by the proteasome complex and antigen translocation into the endoplasmic reticulum/Golgi network using TAP molecules, following the classic MHC class I pathway. PPV-VLPs displaying the OVA epitope follow the TAP-dependent pathway indicating that the PPV-VLPs are processed by the classic mechanism and the antigen bind to new, nascent MHC class I molecules. Moron et al., J. Immunol. 171:2242, 2003. However, the Hepatitis p33-VLPs cross-presentation mechanism operates in both TAP-dependent and TAP-independent fashion in DCs and in the macrophages in a TAP-independent pathway. Ruedl et al., Eur. J. Immunol. 32:818, 2002. Although the processing pathways of these two VLPs have a TAP-dependent component, only the PPV-VLPs are able to protect mice against virus challenge without adjuvant. The CPMV cross-presentation mechanism is presently under investigation. Preliminary studies using the anti-Golgi β-COP antibody show that CPMV particles co-localize with the Golgi apparatus.

It has previously been demonstrated that CPMV carrying neutralizing epitopes induce neutralizing antibodies that protect animals against infectious challenge. Now CPMV may be considered as a candidate for development of dual-purpose vaccines to generate both cellular and humoral responses. CPMV can be genetically modified to carry both CTL and neutralizing epitopes either on the same particle or as a cocktail of epitopes on separate particles. CPMV particles are generally recognized as safe and can be obtained in large amounts by a simple purification protocol, which can easily be scaled up. The ability to further enhance the CTL responses generated by epitopes on CPMV should increase their value as a promising vaccine candidate and provide important data for use of plants as production systems of vaccines.

### Example 27

Efficient Encapsulation and Delivery of Doxorubicin to Tumor Cells by Cowpea Mosaic Virus Particles

Efficient encapsulation of Doxorubicin (DOX) by cowpea mosaic virus particles was achieved through genetic insertion of a high affinity DOX-binding peptide into the viral RNA2 encoding the large subunit of the viral coat protein. The DOX-bound CPMV mutants were stable and effectively induced cytotoxicity in adenocarcinomas of lung and breast cancer cells in vitro as determined by SRB assays. The encapsulated DOX was 5× more effective in inducing cytotoxicity than the free drug at equivalent concentrations in vitro. The DOX loaded virus particles were rapidly internalized in HT29 cells and the intracellularly released DOX was found to localize exclusively in the cell nucleus as judged by the immuno-histochemical techniques.

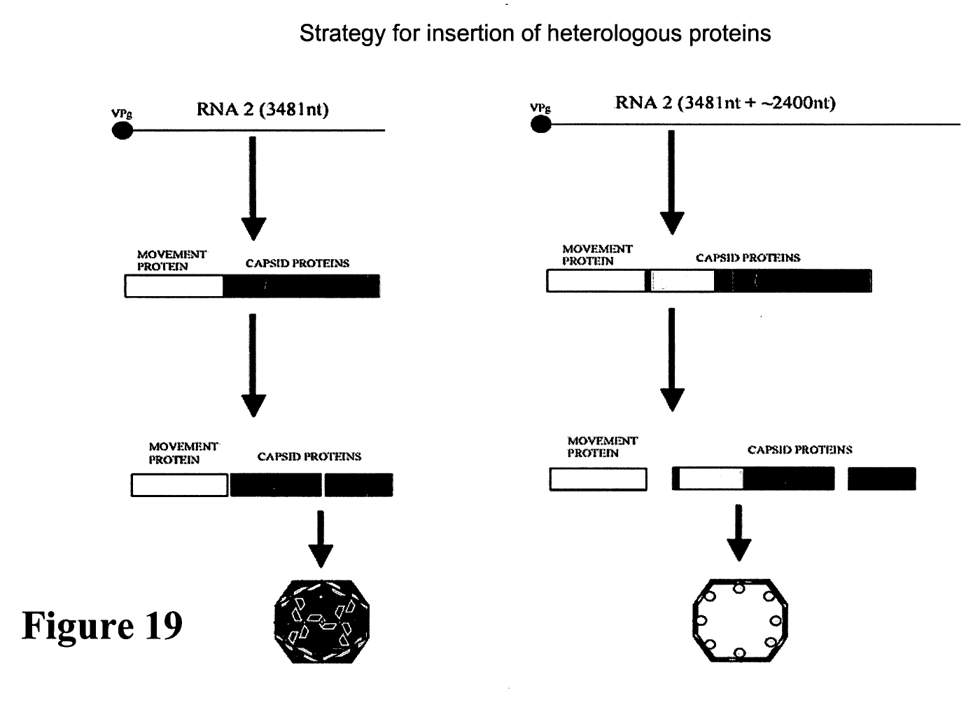

Generation of the construct in the virus capsid interior. Self-assembled cage structures of nanometer dimensions have been used previously (Douglas, Young 1999, 2001) as constrained environments for the preparation of nano-structured materials and the encapsulation of guest molecules for potential applications in drug delivery and catalysis. The interior space within the viral capsid was exploited to accommodate heterologous, non-viral proteins in it. The rationale to present small peptides in the interior of the virus capsid was based upon the differences in the internal volume of the two capsid components of CPMV as a consequence of different amounts of RNA packaged inside the capsid. The genome of CPMV is bipartite and the two RNA molecules are separately encapsidated. The RNA 1 is 5.8 kb in size while the RNA 2 is 3.4 kb in length. The size difference between the two RNA molecules suggests that theoretically there is sufficient room in RNA2 to accommodate foreign proteins of about 2-2.5 kb and still have the particles assembled properly. Earlier studies have established that the viral RNA2 is encoded as a large polyprotein that gets proteolytically processed to generate the coat protein and the movement protein. Therefore, if the foreign protein is expressed as a part of the large subunit of the coat protein then, during the process of RNA encapsidation and assembly, the foreign protein will most likely be assembled and encapsidated within the particles as well. The N-terminus of L proteins folds in the interior of the capsid and proteins presented at this location would likely be packaged inside once the capsid assembles (FIG. 19a).

FIG. 19 shows a schematic representation of the RNA 2 of the CPMV genome to highlight the rationale for insertion of DOX binding peptide in the interior of the virus capsid. On the left is the wt RNA 2 that is translated as a polyprotein and gets further processed by the proteases to form the movement and the coat protein (CP) of the virus. The CP is further cleaved to generate the large and the small subunit of the capsid protein. On the right is the hypothetical insertion mutant in which the heterologous non-viral peptides are engineered (yellow) at the N-terminus of the large subunit of the CP. The N-terminus of the CP after processing gets folded inside the capsid thus encapsulating the foreign peptide in the interior of the virus cavity.

Based on studies that explored the interior of the virus capsid for accommodating heterologous sequences, the N-terminus arm of the capsid was rather extended and might be flexible enough to allow for small insertions without compromising the stability or assembly of the particles. A small 12 aa peptide was inserted that was identified via phage display to bind DOX and other chemotherapeutic drugs like the verapamil, vincristine and vinblastine (Popkov et al., 1998). The peptide (VCDWWGWGIC) was inserted at the N-terminus of the large subunit of the viral capsid. The chimeric particles were propagated in plants, purified and characterized by spectroscopic methods.

### Example 28

Characterization of the DOX Chimera

Since the DOX binding peptide introduces three tryptophan and two cystines residues into the viral sequence, it was expected that the inserted peptide might alter the behavior of the native CPMV particles. Intrinsic tryptophan fluorescence was used to verify these effects.

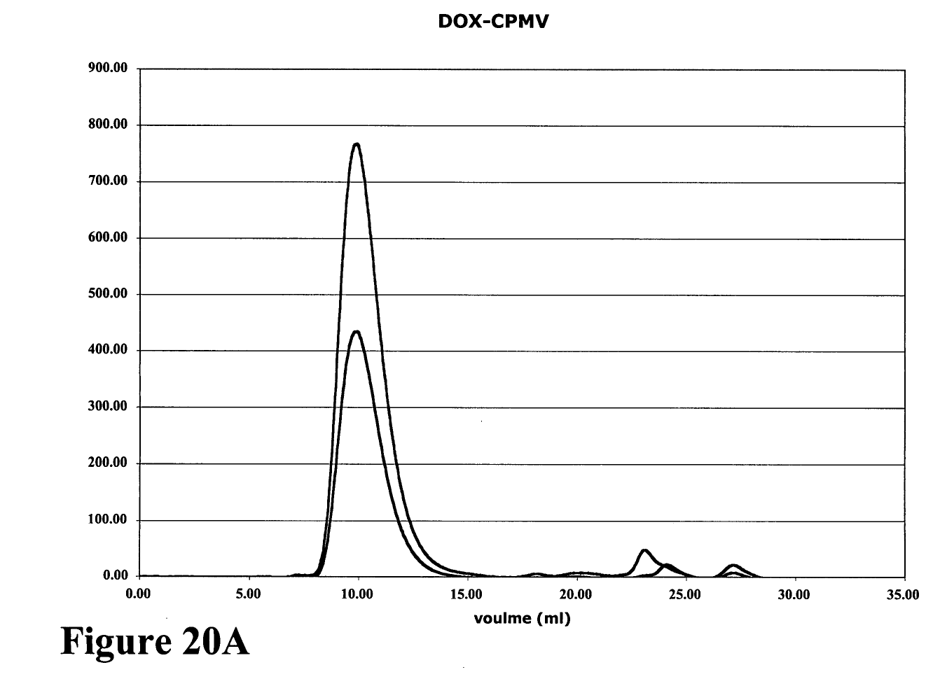

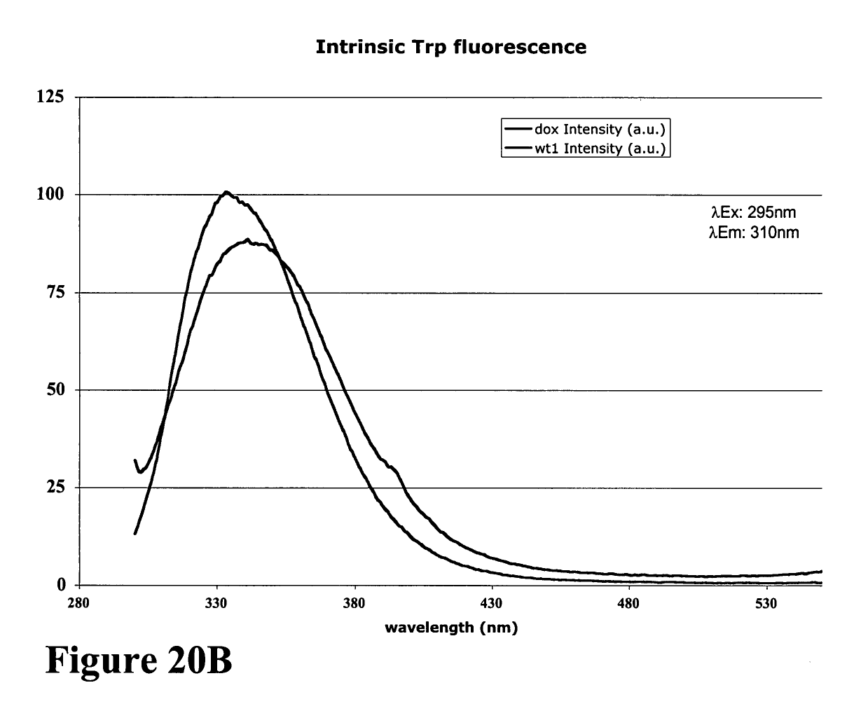

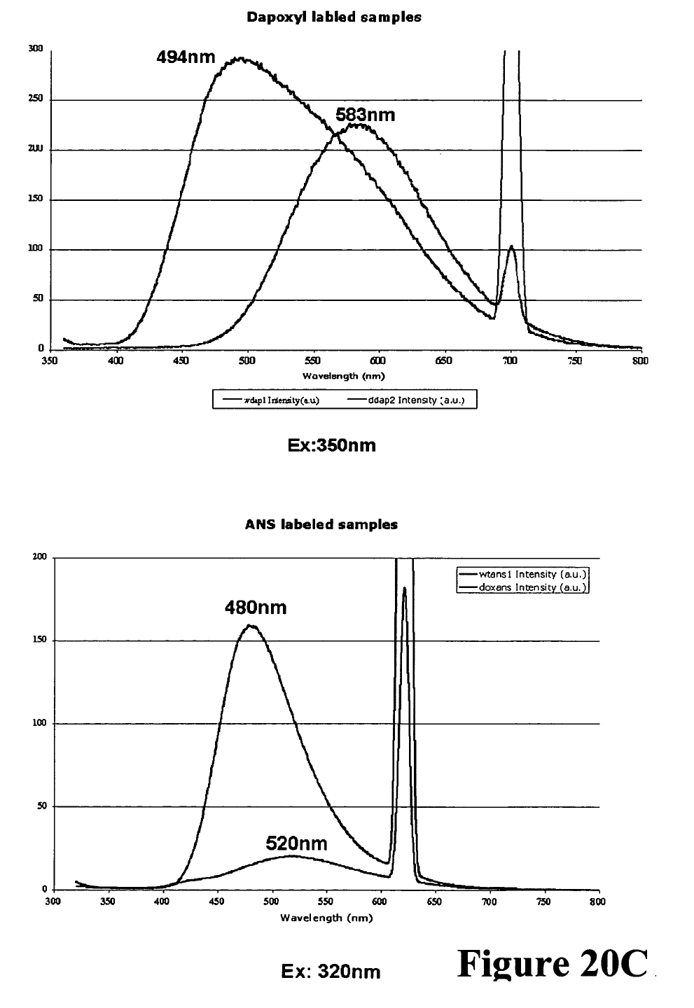

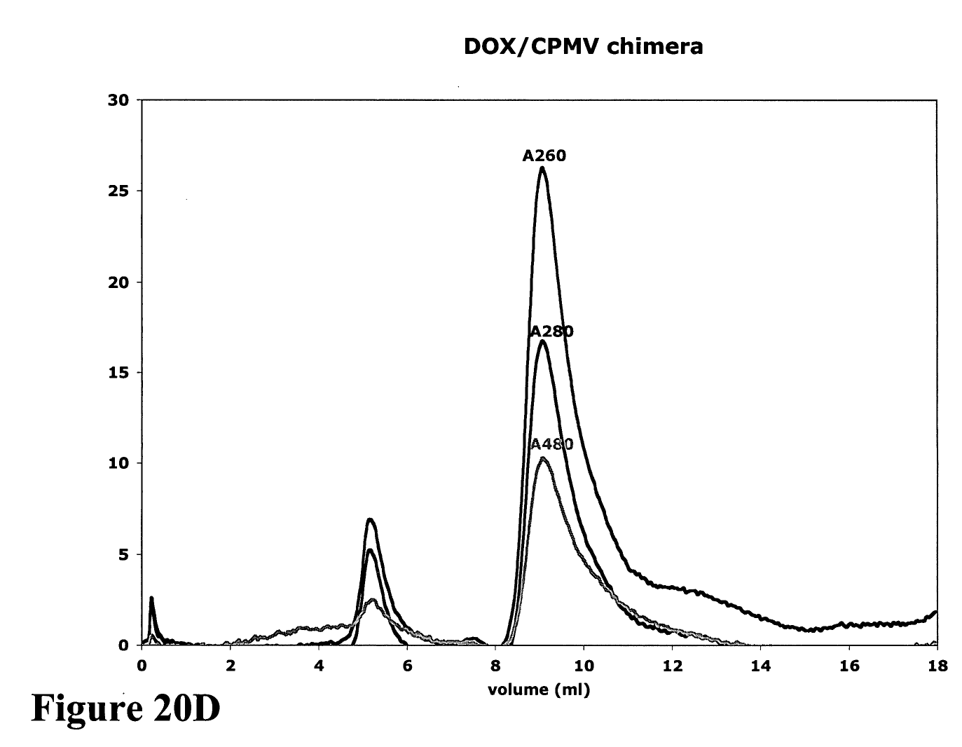

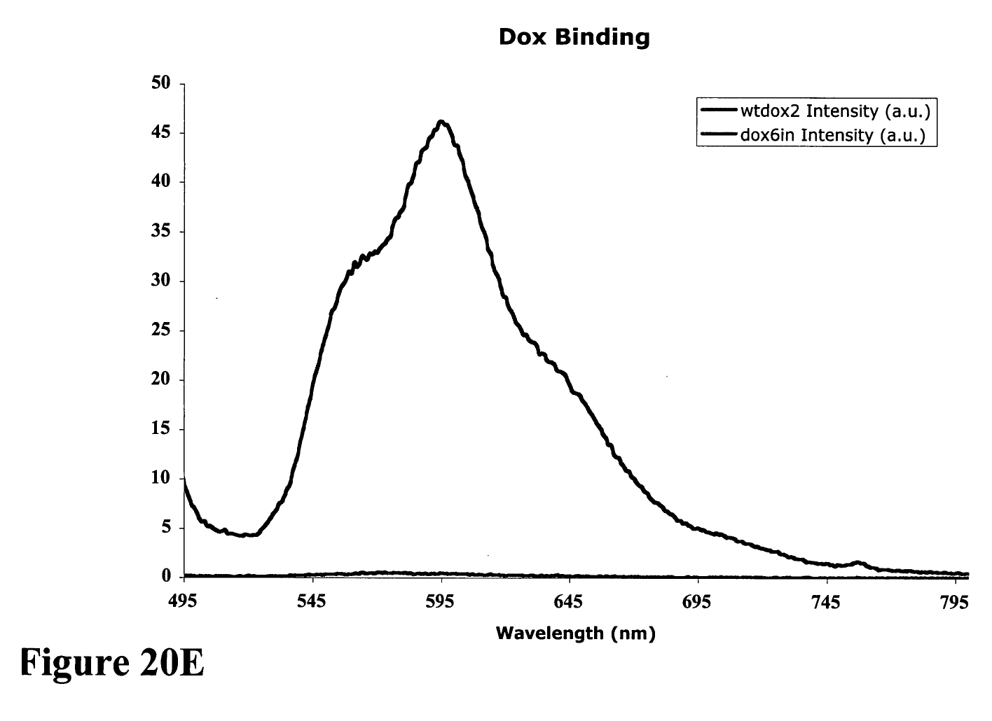

A. Changes in the intrinsic fluorescence of the DOX chimera. As is evident from the results in the FIG. 20A, there is a distinct shift towards the longer wavelengths in case of the DOX chimers in comparison to the wt. CPMV. These results indicate that the physical properties of the chimeric virus particles had changed as a result of the peptide insertion.

B. Biophysical characterization of the DOX chimera. The intactness of the particles as well as its ability to bind the DOX was determined by size-exclusion chromatography, UV-VIS spectroscopy and fluorescence. The particles with bound drug were resolved on a superose 6 column and monitored at 480 nm for the bound DOX in addition to the 260 nm and 280 nm for the viral RNA and the coat protein respectively. A distinct fluorescence associated peak that co-eluted with the virus particles was observed confirming that the particles bound the DOX efficiently. (FIG. 20B).

C. Fluorescence associated changes with the DOX bound samples. A comparison of the fluorescence associated with the wild type (wt) virus with the DOX chimera after they are exposed to the drug revealed that the DOX-CPMV chimera particles bound the drug specifically as seen in their emission spectrum (FIG. 20C). The wt virus does not bind any appreciable amounts of DOX while the chimera-associated emission at 595 nm suggests a more specific interaction of the particles with the drug that is indicative of the presence of the foreign peptide inserted in the virus genome.

D. Labeling particles with conformational specific dyes. ANS and Dapoxyl was used to label the hydrophobic patches in the virus capsid. As seen in the figure, there was a significant increase in the intensity of the chimera with the ANS labeled samples. The dapoxyl bound samples exhibited the same behavior as the ANS ones. In both cases the DOX samples exhibited a similar profile with a significant shift in the blue region of the spectrum confirming the non-polar character of the DOX binding peptide pocket in the virus (FIG. 20D) associated with a simultaneous increase in the overall intensity of fluorescence.

FIG. 20A shows intrinsic tryptophan fluorescence of the wt and DOX-CPMV mutant. The differences in the steady state fluorescence of wt CPMV and DOX-CPMV chimera were determined by measuring their emission spectra (λex: 290 nm, λem: 310 nm). The contribution of the additional tryptophan residues in the insertion mutant (red trace) was reflected by a distinct shift towards the longer wavelengths as compared to the wt CPMV (green trace).

FIG. 20B shows characterization of the DOX-CPMV mutant. The intactness of the DOX-CPMV mutant particles as well as their ability of to bind doxorubicin was demonstrated by resolving the chimera on a size exclusion gel filtration column (Superose 6 Hr 10/30). Co elution of the Dox specific peak measured at 495 nm with the virus indicates the specific binding of DOX by the virus particles.

FIG. 20C shows fluorescence emission spectrum of the DOX loaded CPMV chimera. The purified DOX bound CPMV particles (100 ug/ml) were analyzed for any doxorubicin associated emission by fluorescence spectroscopy. The specificity of DOX binding to the chimera was verified by the presence of a typical emission maximum at 595 nm. The wt CPMV (green trace) showed negligible binding to DOX under identical drug loading conditions described under materials and methods.

### Example 29

Quantitation of DOX Molecules Bound to the Virus

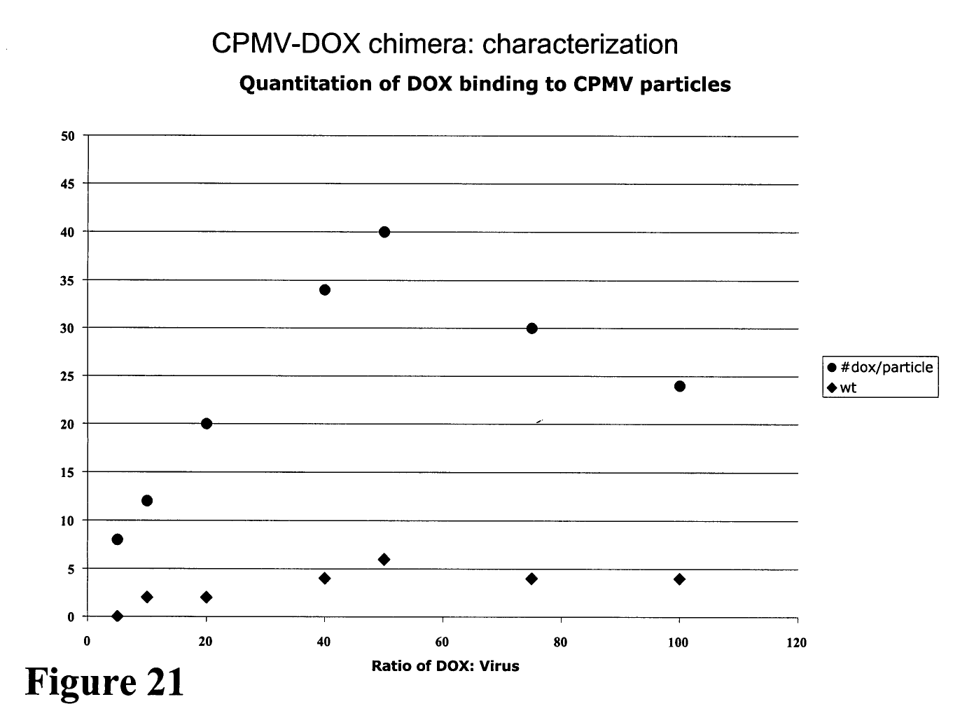

The ability of the DOX-CPMV chimera to bind DOX was further evaluated by incubating the virus with increasing concentrations of the drug. Quantification of the encapsulated DOX by UV/VIS spectroscopy and fluorescence indicated an average of 40 DOX molecules were bound per virus particle (FIG. 21). This number corresponds to 374 μM or approximately 240 ngDOX/mg of virus. Gel filtration chromatography of the DOX bound particles did not show the presence of any unbound, free DOX in the sample. The number of bound DOX molecules was quantified by measuring the absorbance of the bound DOX at 480 nm. Attempts to load increasing amounts of DOX to the particles were not very successful as the virus precipitated out of solution possibly reflecting the increased hydrophobicity or stearic constraints associated with the binding of DOX to the particles. The results in the FIG. 21 give the amount of DOX equivalents accumulated per virus particle.

FIG. 21 shows quantification of Doxorubicin molecules bound to DOX-CPMV chimera. The number of DOX molecules attached to CPMV was determined as a function of increasing molar ratio of DOX to the virus. Stoichiometric analysis was done by measuring the absorption of DOX labeled CPMV at 480 nm (DOX) and the virus (260 nm). The extinction coefficient used for DOX is 1100 m⁻¹. The virus was incubated with different concentrations of DOX for 4 h at room temperature after which the labeled particles were purified by gel filtration chromatography and ultracentrifugation through sucrose cushions (materials and methods). The DOX CPMV chimera could bind a maximum of 40 DOX molecules (green circles) while the wt CPMV under identical conditions bound only 6 of them (red diamonds).

### Example 30

Efficiency of Cell Killing

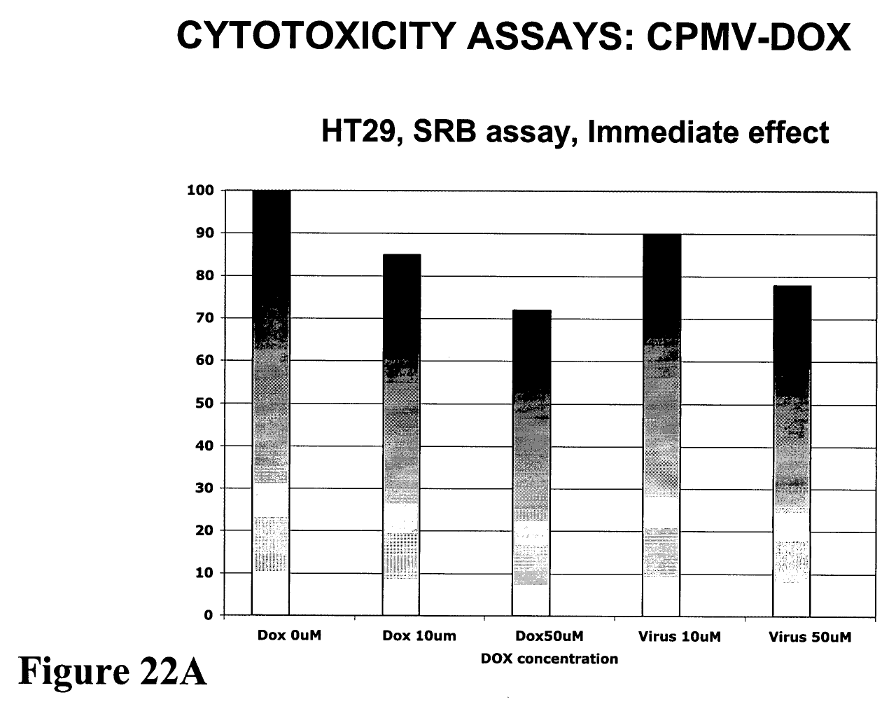

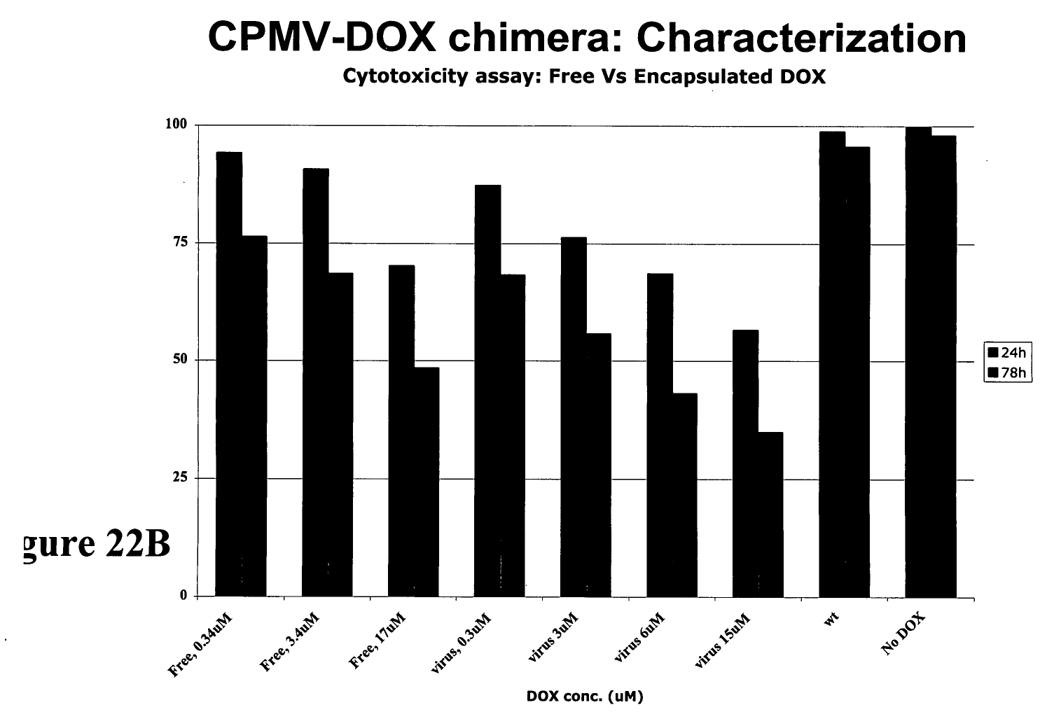

Chemosensitivity of DOX-CPMV chimera. The efficacy of the DOX bound chimera to cause cell cytotoxicity was judged by calorimetric assays using SRB method. Total cell viability was analyzed by staining with SRB and measuring the absorbance at 564 nm. Total cell count was assumed to be linear to the recorded absorbance of the dye. A comparison of the free DOX with the DOX-CPMV chimera showed that at comparable concentrations of 1 ug/ml; 10 ug/ml; 20 ug/ml and 50 ug/ml, the virus-encapsulated DOX was more effective, resulting in greater than 50% cell mortality (FIG. 22).

Another effect that became apparent during the cell viability assays was the overall delayed effect on the cells after the DOX had been removed from the medium. In both cases, with free DOX as well as virus-encapsulated DOX, the increase in the treatment enhanced the overall decrease in cell viability. The delayed effects were more pronounced than the immediate effects and resulted in significantly increased cell death (FIG. 22).

FIG. 22 shows cytotoxic effects of Doxorubicin exposure in HT29 cells as free drug or encapsulated in CPMV particles. Cells were incubated with free DOX or encapsulated DOX for 4 h after which they were allowed to grow in complete medium for either 24 h (green) or 78 h (red). Different concentrations of the virus and the free DOX used in the experiment are indicated.

### Example 31

Delivery and Release of the Drug into the Cells

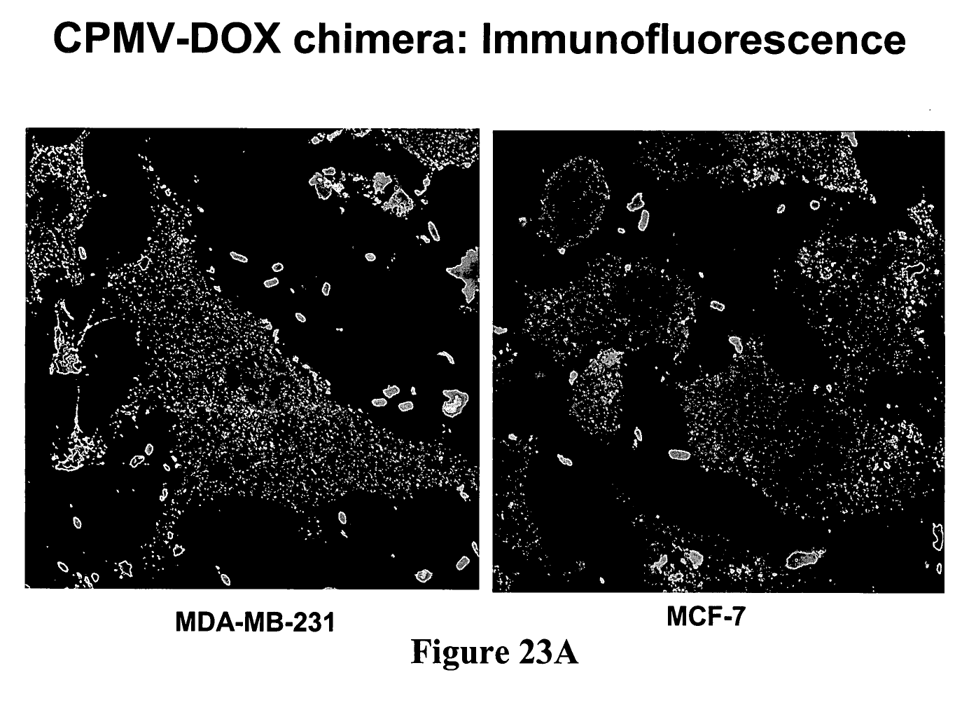

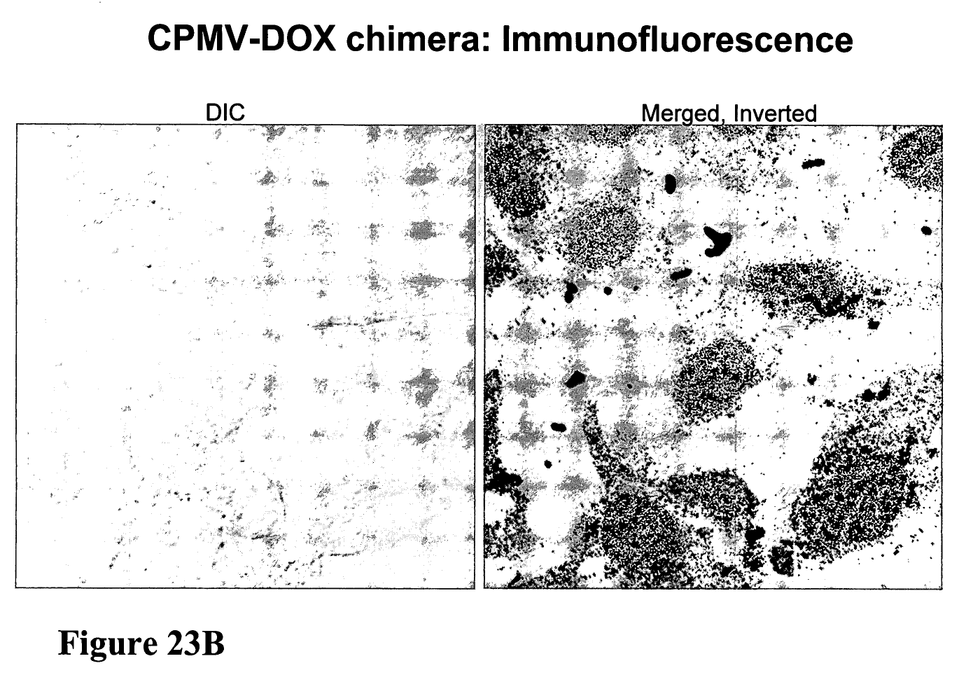

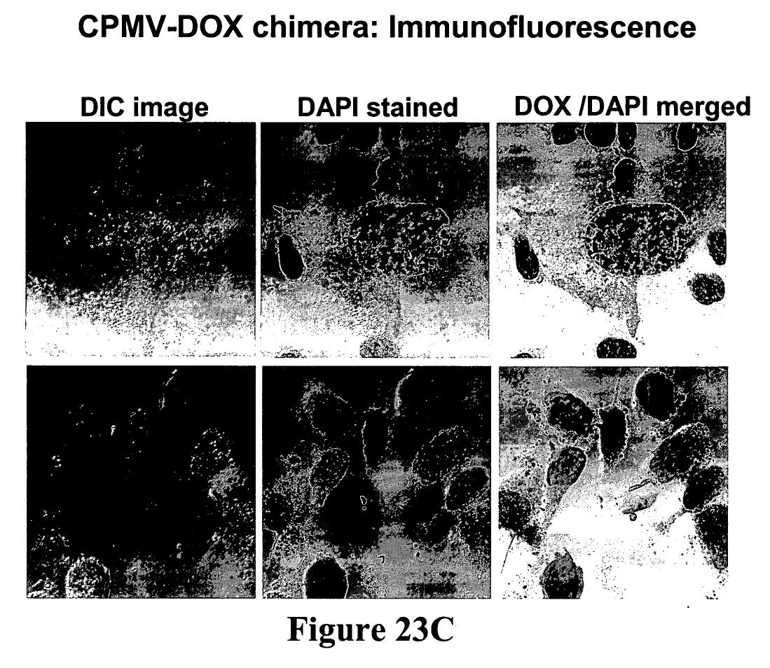

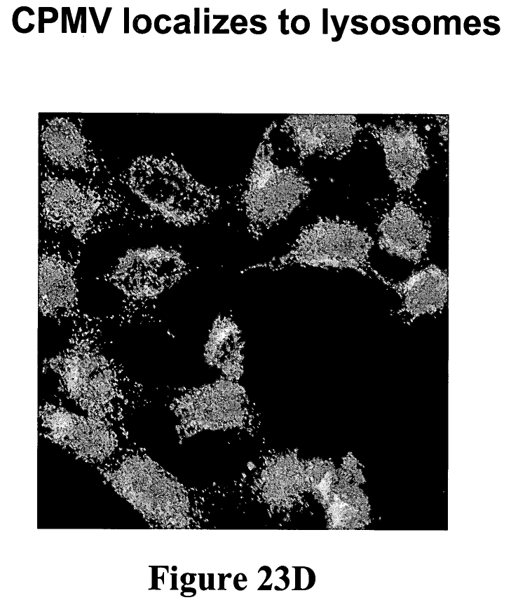

Confocal Microscopy. Incubation of HT 29 and MDA-MB 231 cells with the DOX loaded CPMV did not just result in efficient binding to the cells surface but even internalization within the cells. Immunofluorescence studies using anti CPMV labeled antibodies established that most of the cytoplasm was flooded with the virus 8 h after incubation with the cells. Interestingly, the fluorescence associated with DOX was localized selectively into the nucleus (FIGS. 23A, 23B). No fluorescence associated with the virus was seen in the nucleus. To verify the distribution of DOX in the cell, the nuclei of the treated cells were stained with DAPI and the overlay images of the nuclei with DAPI and DOX were obtained. Most of the DOX was localized in the nucleus and little was seen distributed into the cytoplasm (FIG. 23C).

At this stage it was of interest to determinine if these virus particles were being localized into the cytoplasm or gets specifically compartmentalized within the cell. Co-localization of the virus with lysosome specific markers (anti lamp 1 antibody) revealed that most of the virus could be detected in the lysosomes within 24 h post incubation with the cells. The overlayed images showed a good degree of overlap between the anti CPMV and anti Lamp1 antibodies (FIG. 23D). Presumably, within the lysosomes overtime the virus particles get disassembled/degraded where the low pH and ionic strength of the particles are not favorable for the virus particles to remain intact.

FIG. 23A shows confocal microscopic analysis of cells treated with the DOX-CPMV chimera. Two different breast cancer cell lines, MDA-MB-231 and MCF-7 cells treated with DOX loaded CPMV particles for 4 h at 37 C were permeabilized and fixed with paraformaldehyde. The fate of the virus and the Dox were evaluated by using an anti CPMV antibody and a FITC conjugated secondary antibody. The virus-associated fluorescence (green) was detectable throughout the cytoplasm. The distribution of DOX was measured by excitation of the cells at 595 nm that showed most of the drug present in the nucleus of the cells (red).

FIG. 23B shows Intracellular distribution of DOX bound CPMV particles. MDA MB 231 cells treated with Dox loaded particles reveal that most of the DOX is delivered to the nucleus of the ells while the virus particles are seen only in the cytoplasm. No virus-associated fluorescence was seen in the nucleus indicating that virus particles are excluded form the nucleus.

FIG. 23C shows Immuno-flouescence analysis of DOX treated MDA MB 231 cells. The nuclei of the treated cells were stained with DAPI and then co-localization of DOX associated fluorescence and the DAPI was determined in overlay images. Almost complete co-localization of two fluorescence signals was observed in most cells.

FIG. 23D shows localization of CPMV to lysosomes. The distribution of CPMV in MDA MB 231 cells was determined after incubation with the virus particles for 8 h at 37° C. The cells were stained with a polyclonal anti CPMV and a monoclonal anti lamp1 antibody. The secondary antibodies were either FITC conjugated (for CPMV) or Rhodamine conjugated (for lamp 1) for detection of virus specific and lysosomes specific markers. The overlay of virus specific signal and the lamp1 specific marker for lysosomes showed a good degree of co-localization of the virus into the lysosomes.

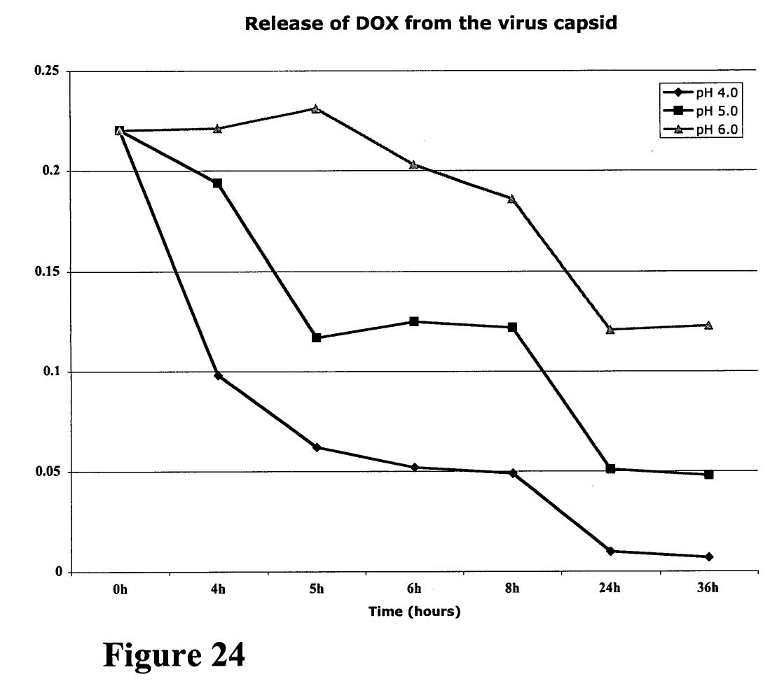

The release of the DOX from the particles under acidic conditions was demonstrated in vitro wherein the DOX loaded virus particles were incubated at acidic pH and the release of the bound DOX was monitored over a period of time by UV-VIS spectrophotometry. Most dramatic release was observed at pH 4.0 wherein more than 70% of the DOX comes off of the particles (FIG. 24) 6 h after incubation at that pH. Changes in the absorbance for DOX were less dramatic at pH 5.0 yet it was noteworthy that 40-75% of the DOX was released between 8-24 h time point. At pH 6.0, no significant loss of DOX from the capsid was observed even after 36 h (FIG. 24). The pH of the lysosomes is known to be about 5.0. The DOX is loaded into the particles at high pH (8.0) and high ionic strength, 250 mM NaCl. It is likely that the particles do not encounter the same ionic strength and pH in the lysosomes, which most likely contributes to the release of the DOX from the virus.

FIG. 24 shows the release of DOX from CPMV particles as a function of pH. More than 50% of the DOX was released from the virus particles in 6-8 h at pH 5.0 (green) while at pH 4.0 (blue) the result was more dramatic. No significant release of DOX was seen at pH 6.0 at similar time points (orange).

### Example 32

Intactness of the CPMV Nanoparticles

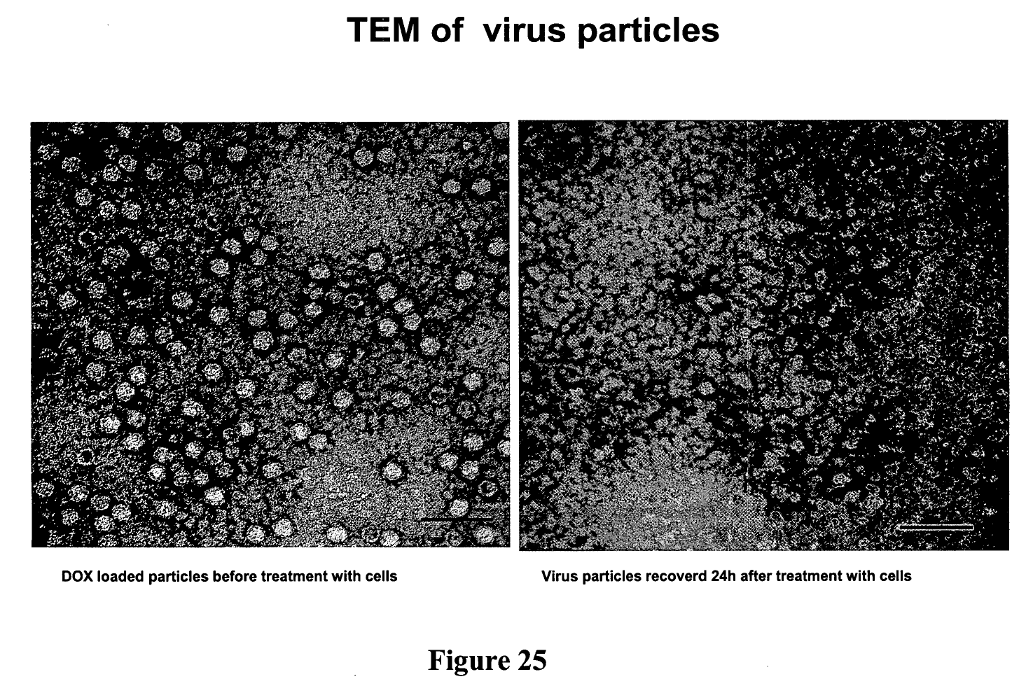

TEM of the DOX loaded particles revealed a regular morphology, similar to the wt particles. Observation of the virus particles recovered from the cells 24 h post treatment however, did not show any intact particles. (FIG. 25) SEC of the same sample confirmed the earlier observations with the TEM, as more than 80% of the particles were found degraded.

FIG. 25 shows intactness of CPMV particles before and after the treatment with cells. 25A: DOX loaded CPMV particles purified after the loading reaction. More than 80% of the particles were intact and had regular wt particle morphology. Few empty articles with dark interiors were also observed. 25B: The virus particles recovered from the cells after 24 h. The cells were gently lysed in PBS and detergents and the total protein recovered after centrifugation. Examination of the protein pellet did not show any intact particles.

### Example 33

Targeted Delivery of CPMV Nanoparticles into Tumor Endothelial Cells

With the availability of a range of natural and synthetic scaffolds, the field of nano-medicine has witnessed a substantial surge in the area of novel nanodevices and their use for targeted drug delivery. Several virus-based platforms, including CPMV have generated a lot of interest and attention because of their small size, the ability to incorporate and display multiple functionalities at various locations on its capsid and the availability of internal space within the virus cavity. Unlike the other popular virus based scaffold, like CCMV, CPMV however cannot be assembled in vitro to generate empty particles for encapsulation studies, but its ability to tolerate small insertions in the RNA 2 of its genome have resulted in the expression of small proteins in the interior of the virus capsid (GFP). In this study the ability of these particles to entrap and deliver anti cancer drugs like DOX to tumor cells was explored. The results demonstrated that virus particles bound the DOX efficiently (374 μM/mg of the virus) and that the encapsulated DOX was cytotoxic to MDA-MB-231 and HT 29 adenocarcinoma cells. Further, the encapsulated DOX was at least 5× more effective than free DOX at equivalent concentrations in causing 20% more cell death in vitro measured both, as immediate and delayed overall effects.

There is a plethora of literature available that discusses the applications and the utility of liposomes derivatized with various modifications to allow for efficient delivery of the DOX to cells. Alternatively, there are other antibody-based methods that have been currently used to demonstrate targeted drug delivery and regression of tumors in mice. Virus particles offer advantages over both these systems in terms of flexibility for drug targeting, efficacy of delivery, size of the particles and the body's immune response to these particles. The most popular small scale delivery systems currently available and under active investigation are the polymer-based systems that carry DOX or other similar anticancer agents to the target tissues. The main advantages of developing viruses to carry the drugs over the lipsomes or other synthetic particles for delivery are related to biocompatibility with body's immune responses, complete lack of toxicity associated with synthetic nanoparticles like the silica dust and the dendrimers, and negligible uptake/retention by the reticulo-endothelial system.

The particles were designed to present a high affinity drug binding peptide in the interior of the particles. For efficient and directed encapsulation, the Dox binding peptide was inserted at the N-terminus arm of the large subunit of the coat protein that folds in the interior of the capsid to ensure that the inter-subunit contacts will not interfere with the assembly of the capsid. This peptide thus serves as a molecular anchor for binding of DOX. The expectation from such a design was two fold, one, the hydrophobic DOX-binding peptide if displayed in the interior of the virus particles would bind the drug more efficiently and keep the virus particles stable as opposed to it being presented on the surface on the surface which may have caused virus insolubility problems. Secondly, the encapsulated drug would have a greater likelihood of being protected from the proteolytic degradation in cell culture supernatants and related biological fluids if administered in vivo. The DOX binding peptide contains two cysteines and separated by seven amino acid residues. This distance is considered to be highly preferable to for the formation of an internal disulfide bond suggesting that the peptide most likely has a cyclic structure. Previous studies have also indicated that the peptide has not only some functional but also some structural analogy with the drug binding sites within the P glycoprotein (Popkov et al., 1998). In the same paper it was indicated that the synthetic peptide was able to compete at nanomolar concentrations range with the phage for DOX binding suggesting that the drug binding activity of the peptide is the intrinsic activity of the peptide rather than that of the phage particle expressing the peptide.

For loading the DOX into the interior of the CPMV capsids, an incubation and binding step is required. On an average, a maximum of 40 DOX particles could be loaded in the virus capsid based upon the absorption spectroscopy of the bound sample. A lot of specific and hydrophobic interactions probably contribute to this binding effect. Similar interactions were shown to play a role in binding of methotrexate (MTX), another anticancer agent to the polyomavirus capsids (Abbing et al., 2004). Theoretically, it was expected that the CPMV capsid to bind at least 60 copies of DOX molecules considering that each peptide binds a single DOX molecule, however, the inability to load more DOX molecules into the capsid may reflect the stearic influences or an alteration in the pentamer interactions that favor disassembly of the particles at greater loadings of the drug.

The intracellular fate of the DOX loaded particles was also investigated by immuno-fluorescence methods. The results showing internalization of the virus even though unexpected are reminiscent of several other works where in the researchers have observed that virus particles tend to get compartmentalized into the lysosomes. These findings also agree well with a cell's physiological response to limit the foreign matter into compartments where they can be degraded. Further, the internalization of an animal cell by a plant virus lacking the appropriate receptors for entry even though unexpected is not entirely surprising given the fact that most of these tumor cells are hyper-permeable. In fact, it may even be an advantage for the particles when administered in vivo as the vascular system in tumor cells is very leaky (Netti et al., 1999), so, it may not be unreasonable to expect that the DOX bound virus will find gain access to the tumor cells subsequent to extravastating into the tumor from the circulation provided it had a targeting ligand to a surface receptor on the tumor cells.

The release of DOX from the particles under acidic conditions usually encountered in the lysosomes was demonstrated in vitro. Based on these results, the low pH and the ionic strength of the lysosomes might play a role in the release of DOX within the cells.

Besides the ability to bind and deliver DOX intracellularly, the effectiveness of the encapsulated DOX to induce cytotoxicity in cells was studied. Cytotoxicity and cell viability analysis using the SRB method suggested that the encapsulated DOX is more effective than free DOX in reducing cell survival at equivalent concentrations. Most studies with liposomes encapsulated DOX have found similar results suggesting that the hydrophobic drugs are most effective when they are encapsulated and invariably more stable and effective than the free drug at similar concentrations. The chemosensitivity results demonstrated that the cpmv-DOX was 5× fold more potent than the free DOX in a transient exposure assay at 3 μM-15 μM concentration range. There are many examples in the literature that indicate that the targeted, encapsulated DOX is effective within a range of 0.3-72.4 μM. The virus encapsulated DOX without a targeting ligand showed a much higher increase in the potency of the drug that is likely to improve substantially if targeted to a receptor on the tumor cells.

In summary, the usefulness of small, 30 nm icosahedral virus particles has been demonstrated for drug encapsulation purposes that should find applications in the field of targeted drug delivery. The particles entrap the drug efficiently, rapidly bind to and internalize within tumor cells in vitro and can deliver and release the DOX intracellularly. It is noteworthy that coupling of DOX to the virus does not result in a loss or alteration in its biological activity. The delivered DOX is effective in inducing cytotoxicity to the cells in transient exposure assays in which it was found to be more effective than the free DOX at equivalent concentrations. Overall, these results indicate the potential utility of these virus-based nanoparticles for drug delivery applications. The next step will be to incorporate a targeting ligand on the exposed surface of the virus capsid so that the encapsulated DOX can be selectively targeted to cancerous tissues and circumvent the side effects on healthy ones.

### Example 34

Materials and Methods

Chemicals: Doxorubicin was obtained from EMD Biosciences. All other chemicals were purchased from Sigma-Aldrich. The cell culture media and related chemicals were obtained from Invitrogen, Carlsbad, Calif.

Cell Culture conditions: Human colon carcinoma cell line HT29 and the breast cancer cell lines, MDA-MB-231 and MCF-7 were obtained from the American Type Culture Collection. The cells were maintained in Dulbecco's Modified Eagles Medium with 10% fetal bovine serum, 0.01% penstrep and 5% glutamine. The cells were cultured with complete medium at 37° C. in a humidified atmosphere of 5% CO₂ in air. For all experiments, cells were harvested from sub confluent cultures using trypsin and were resuspended in fresh complete medium. Cells greater than 90% viability as determined by trypan blue exclusion assay were used for experiments.

Propagation of the Virus in Plants: The primary leaves of cowpea seedlings were mechanically inoculated with 10 ug each of cDNA plasmids encoding RNA1 (pCP1) and RNA2 (pCP2). The initial inoculum of native CPMV was extracted from infected leaves with 0.1 M potassium phosphate (pH 7.0) (phosphate buffer) 7 days post-infection. Typically, 50 plants were infected with the plant extract, and the symptomatic leaves were harvested after three weeks. The virus was isolated according to the standard protocol [3] with minor modifications. Further purification of the virus on sucrose gradients followed by chromatography was done as previously described.

Generation of DOX-CPMV chimera: Site-directed mutagenesis of pCP2 (coding for RNA2 of CPMV) to generate DOX-CPMV mutants was carried out based on established protocols [13]. Synthetic oligonucleotides corresponding to the DOX binding peptide (VCDWEYWCG) were designed for site-specific insertional mutagenesis at the N-terminus of the large subunit. The peptide was inserted 20-21 residues downstream of the first methionine of the large subunit at the Nsi I site in the pCP2 clone. The clones were verified by sequencing and inoculated on plants with pCP1. The plants were grown for three weeks before harvesting. The purified virus was resuspended in 0.1M-phosphate buffer, 250 mm NaCl (pH7.5), 10 mM TCEP.

Preparation of DOX-bound CPMV particle: DOX was encapsulated in the CPMV particles by incubating the purified virus with a concentrated stock solution of Doxorubicin (10 mg/ml). The virus DOX solution was mixed gently and allowed to sit at room temperature for 4 h before purification of the DOX bound particles from the unbound free DOX. The virus was purified from excess DOX by gel-filtration chromatography and high-speed ultracentrifugation through sucrose cushions as described before.

Characterization of DOX-CPMV Chimera:

A. Size exclusion chromatography of DOX-CPMV chimera. Gel filtration chromatography experiments were carried out as described before. 100 μl of 2 mg/ml of the DOX chimera was resolved on a superose 6 HR10/30 column using the AKTA explorer chromatography instrument.

B. Fluorescence spectroscopy of DOX CPMV chimera. To investigate the interaction between the DOX and the virus particles, steady state fluorescence measurements were taken using wt CPMV as control. The intrinsic tryptophan fluorescence was measured by excitation of the virus sample (50 ug/ml) at 290 nm and measuring the emission spectrum over a range (300-600 nm). The emission scans of the wt CPMV were used as controls.

C. Quantitation of DOX molecules bound/CPMV particle. Five milligrams of virus was incubated with different molar excess (1-100×) of Doxorubicin HCL (Calbiochem) in phosphate buffer at room temperature (pH8.0) in a total reaction volume of 500 μl. The reactions were incubated for 4 hours at room temperature after which, the excess dye molecules were removed by a combination of gel filtration chromatography and ultracentrifugation through a 30% sucrose cushion. The derivatized virus was recovered by ultracentrifugation and analyzed by size exclusion chromatography. The amount of DOX attached was determined by measuring the absorbance of DOX loaded virus samples at 480 nm with a spectrophotometer. Virus concentration was measured by determining the absorbance at 260 nm. Each data point was obtained from the average of three independent, parallel reactions that were repeated at least three times. The typical variation was 5-10%. A standard curve for DOX absorbance was generated by measuring the absorbance of free DOX at 480 nm over a range of concentrations from 1 ng/ml-1 mg/ml). The amount of DOX loaded in the virus particles after the binding reaction was determined empirically for the standard curve thus generated.

Cytotoxicity assays: The cytotoxic effects of the free or virus-encapsulated DOX on the cells were assayed calorimetrically by the SRB method. Samples containing 20,000 HT29 or Hela cells in 100 ul aliquots were plated onto 96 well microtiter plates. The culture plates were incubated for 24 h at 37° C. and 5% CO₂ after which the medium in each well was replaced with 100 ul of serum free antibiotic free medium containing various concentrations of free or virus-encapsulated DOX. Four different concentrations of the DOX-CPMV (1 ug, 10 ug, 25 ug and 50 ug/ml) were tried. Each treatment was repeated in six wells and three independent experiments were performed. The cells were incubated with DOX or DOX-CPMV for 4 h after which complete medium lacking the drug/virus was added to each well. At this point the cells were fixed immediately or were allowed to grow in complete mediun free of the treatment for 24 h or 78 h after which they were processed for cell viability assays or for immuno-fluorescence analysis. The cultures were fixed by gently layering 25 ul of ice cold ice cold 50% TCA on top of the growth medium in each well to produce a final concentration of 10% TCA. The cultures were incubated for 1 h at 4° C. after which they were extensively washed, air dried and analyzed by SRB staining. The absorbance for SRB at 564 nm was determined using an optimax microplate reader (Molecular Devices, Sunnyvale Calif.). It was assumed that the cell number is proportional to the level of SRB staining.

Immunofluorescence and TEM analysis: The intactness of virus samples after the DOX reaction was verified by TEM analysis. DOX bound samples at a concentration of 1 mg/ml in 100 mM potassium phosphate, pH 7.0 (phosphate buffer) were placed on glow discharged carbon film and stained for 1 minute with 2% uranyl acetate. The grids were observed under a Philips CM120 microscope operated at 100 kV at a magnification of 45000×. The micrographs were recorded under minimal dose conditions (<20e-/Å²) on Kodak s0-163 film using a nominal defocus of ˜1.0 μm and scanned to a final resolution of 21 um on a Zeiss SCAI scanner.

### Example 35

Cowpea Mosaic Virus Particles are Efficiently Targeted to Tumor Cells

In contrast to other synthetic scaffolds, virus nanoparticles represent a unique way to target and deliver therapeutic compounds to cells of choice while retaining the advantages of a rational, structure-based designed enhanced polyvalent effect of the presented epitopes. CPMV based scaffold has been developed for targeting and delivery of therapeutic compounds to tumor vasculature. The 30 nm nanoparticles functionalized with Flt-1 receptor homing peptides were designed to encapsulate doxorubicin (DOX) and release at the target site. Our results demonstrate specificity of tumor targeting and delivery of DOX bound virus nanoparticles on tumor growth and angiogenesis in human colon carcinoma and breast cancer cells in vivo.

There are a variety of approaches available to target therapeutics directly to tumors. Theses range from antibodies binding to tumor antigens, ligands directed to tumor vasculature and synthetic nanoparticles that combine the targeting ligands and small molecules for cancer treatment (Baker et al., 2003). The advent of phage display and other types of peptide display technologies have also facilitated the discovery of tissue specific ligands that can be used to target tumors. The ability of such drug-binding and VEGFR-1 and VEGFR-2 homing peptides presented on engineered CPMV particles to specifically target tumor vasculature and inhibit angiogenesis has been evaluated.

### Example 36

Generation of FLT-1 and KDR Targeting Chimeras

The rationale for the development of tumor targeting particles is based upon inhibition of angiogenesis and therefore targeting ligands responsible for tumor blood vessel formation and proliferation becomes an obvious choice. The vascular endothelial growth factor receptor (VEGFR) was chosen as a target. There are two growth factor receptors known that play critical role in angiogenesis, the Flt 1, also known as the VEGFR1, and the KDR1, also called the VEGFR2 receptor and are over expressed in almost all types of cancer cells. Particles have been designed that can both, home specifically to tumor cells expressing the two receptors and can also facilitate their uptake into tumor cells. The expectation is that such particles should be able to target specific tumor cells and after internalization into the cell cytoplasm, can deliver their therapeutic payload more efficiently.

In the studies described below two CPMV chimeras have been developed that target either the VEGFR1 (WHSDMEWWYLLG) or the R2 receptor (ATWLPPR) by incorporating short peptides that target the respective receptors. An et al., Int J Cancer 111: 165-73, 2004. The two peptides were identified by phage display and inserted into the exposed loop on the external surface of the viral coat protein. Mousawi et al., J Biolog. Chem. 276: 46681-46691, 2003; An et al., Int J Cancer 111:165-73, 2004. This design preserves the ability to bind and encapsulate DOX in the interior of the virus capsid. We evaluated the ability of these chimeras to specifically bind their cognate receptors in vitro and in animal models using SCID mice that have xenografts of HT29 and MCF 7 melanomas.

Site directed mutagenesis approach was used to engineer the CPMV chimeras expressing short peptides that exhibit VEGF receptor binding affinity to vegf receptor-1 (Flt 1) and vegf receptor 2 (KDR1) in the EF loop of the large subunit of the viral coat protein. The chimeras were propagated in plants, purified and characterized by spectroscopic and chromatography methods.

### Example 37

Binding and Competition Assays Using ELISA

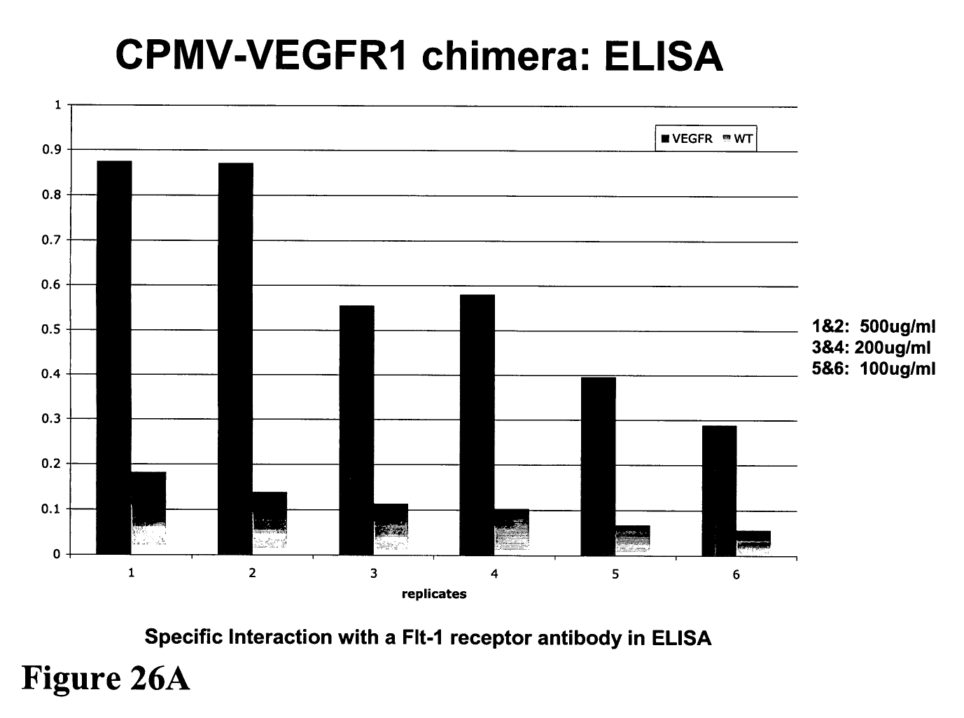

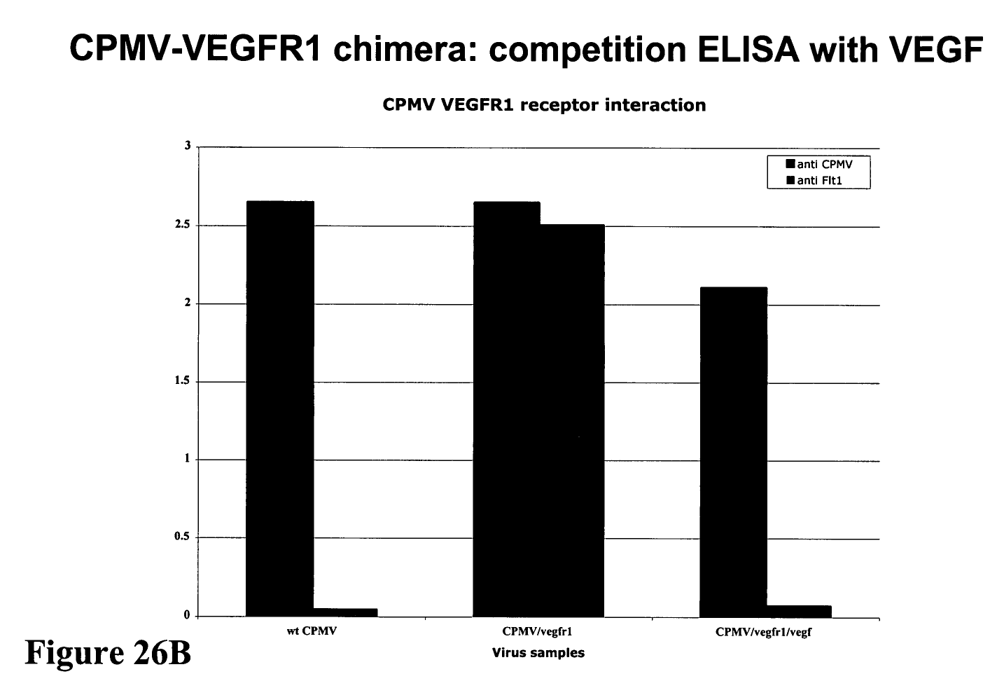

The VEGFR1 and VEGFR2 chimeras were also tested for binding to their respective receptors in ELISA assays. Both chimeras showed specific binding to their cognate receptors while the wt CPMV did not show any detectable binding to these receptors (FIG. 26A). In addition, the binding of the VEGFR1 chimera to the Flt-1 receptor could be competed out significantly by extrinsic addition of the growth factor VEGF to the wells and vice versa (FIG. 26B). This data suggests that the virus may be competing with the growth factor to bind to the receptor and therefore may represent an interesting approach to down regulate proliferation of new tumor vessels by sequestering the receptor.

FIGS. 26A and 26B show interaction of CPMV-VEGFR1 chimera with a Flt-1 receptor antibody in ELISA. FIG. 26A shows the accessibility of the targeting peptide on CPMV capsid was determined in ELISA tests. The plates were coated with Flt1 receptor specific antibody and used to trap the virus particles expressing the vegfR1 peptide. The virus was washed off after 1 hr and the bound virus was detected using HRP conjugated secondary antibody. Wt CPMV shows some background at higher virus concentrations. FIG. 26B shows the VEGFR1-CPMV interaction can be competed with VEGF, the physiological ligand for the receptor, as detected by anti Flt-1 antibody.

### Example 38

Immunofluorescence

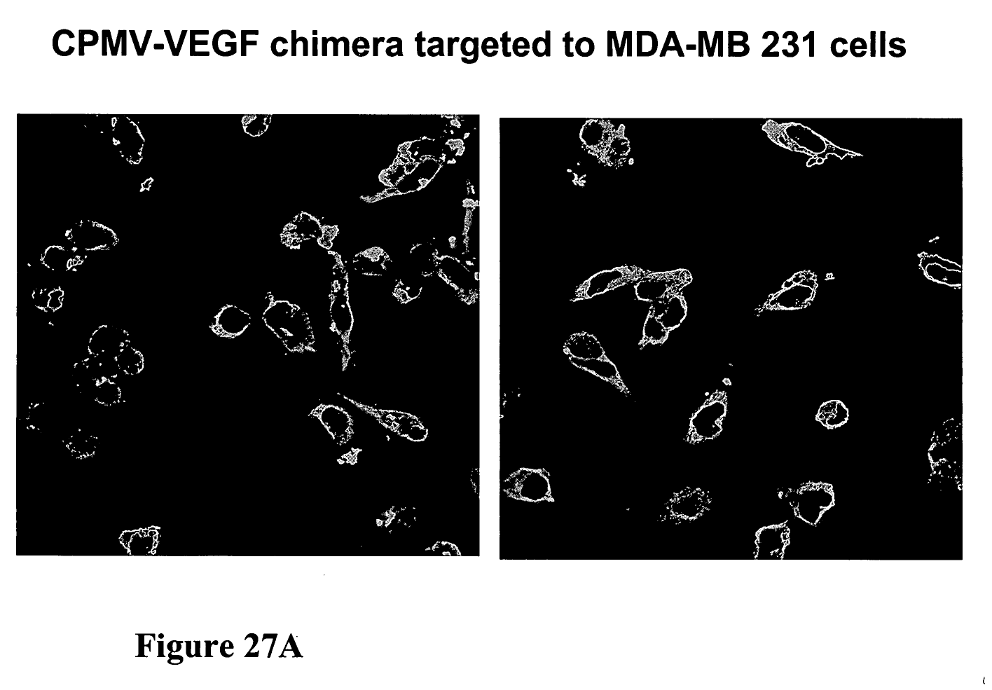

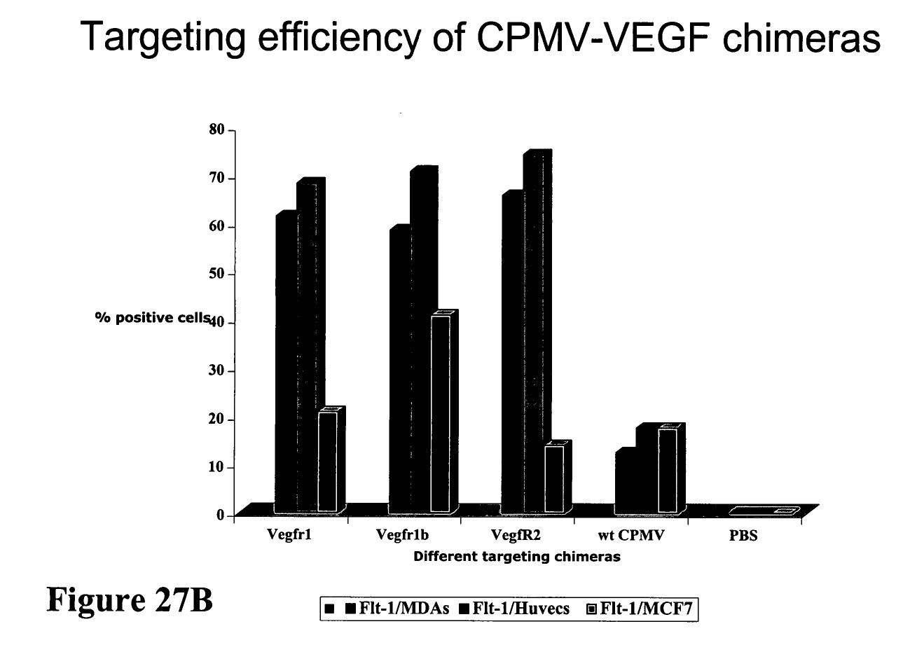

VEGFR1-CPMV chimeras were tested for their ability to bind the Flt-1 receptor in HUVECs using immunofluorescence and flow cytometry. Co localization of the virus particles with Flt-1 specific antibody suggested a strong interaction of the virus with the VEGFR1 receptor (FIG. 27A) after 30 minutes of incubation with the cells. Quantitative estimation of the binding events as determined in FACS assays corroborated the same results indicating more than 65% of binding events related to specific localization of the virus to the flt-1 receptor. (FIG. 27B).

FIG. 27A shows CPMV-VEGF chimera targeted to MDA-MB 231 cells. FIG. 27B shows a FACS analysis of targeting efficiency by the CPMV-VEGF chimeras in different tumor cell lines. The percentage of targeting efficiency was calculated from the number of positive cells that bind the receptor as quantified by flow cytometry.

### Example 39

In Vitro Angiogenesis Assays

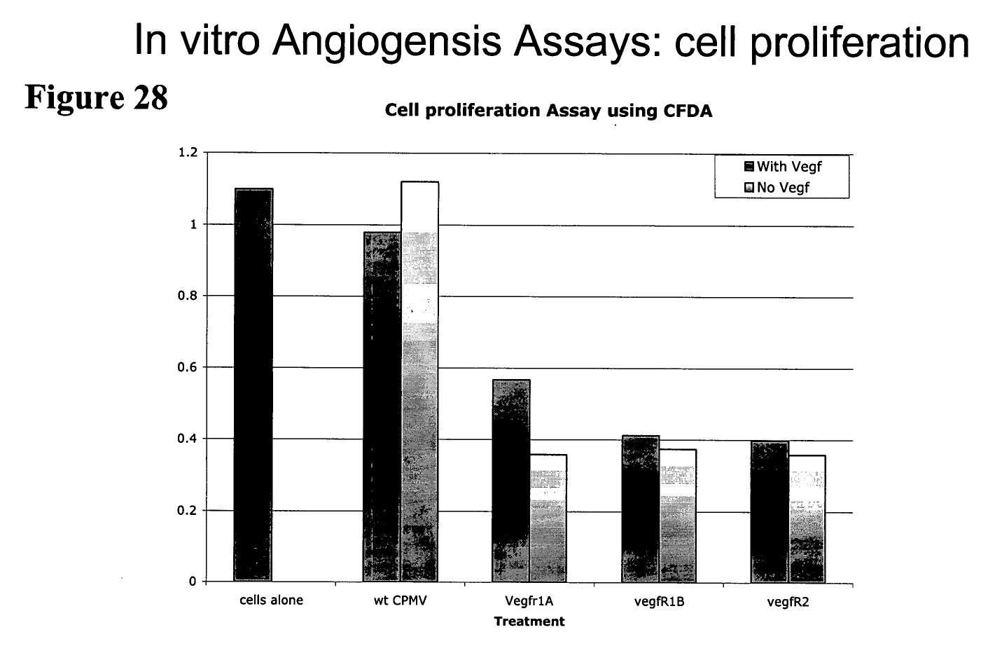

Cell proliferation assay using CFDA. The effects of Vegf CPMV chimeras on endothelial cell proliferation was assessed in HUVECS cells stimulated with vegf using the incorporation of CFDA (carboxyfluorescein diamine acetae, Molecular Probes) dye as a measure of cell division and growth. HUVECS were stimulated with 10 ng/ml of vegf and the treated with various vegf receptor-targeting chimeras. As shown in the FIG. 28, the chimeras inhibited cell proliferation of HUVECS in a significant manner with an IC₅₀ of 30 ug/ml. At a concentration of 30 ug/ml the vegfR1 chimeras inhibited cell growth by 50-60%. At the same concentration, the wt CPMV did not affect cell growth/proliferation.

FIG. 28 shows cell proliferation in in vitro angiogensis assays. HUVECS were starved for 24 h in serum free glucose free medium after which they were exposed to VEGF and/or VEGFR1chimera. Wt CPMV was used as a control/. After 48 h incubation, CFDA was added to the medium, grown for 24 h and the dye incorporation was recorded as an indication of rate of cell proliferation. In the presence of VEGF, one sees better inhibition of cell proliferation. At a concentration of 30 μg/ml, the virus inhibited VEGF induced proliferation by 50-60%. The wt CPMV did not affect cell proliferation.

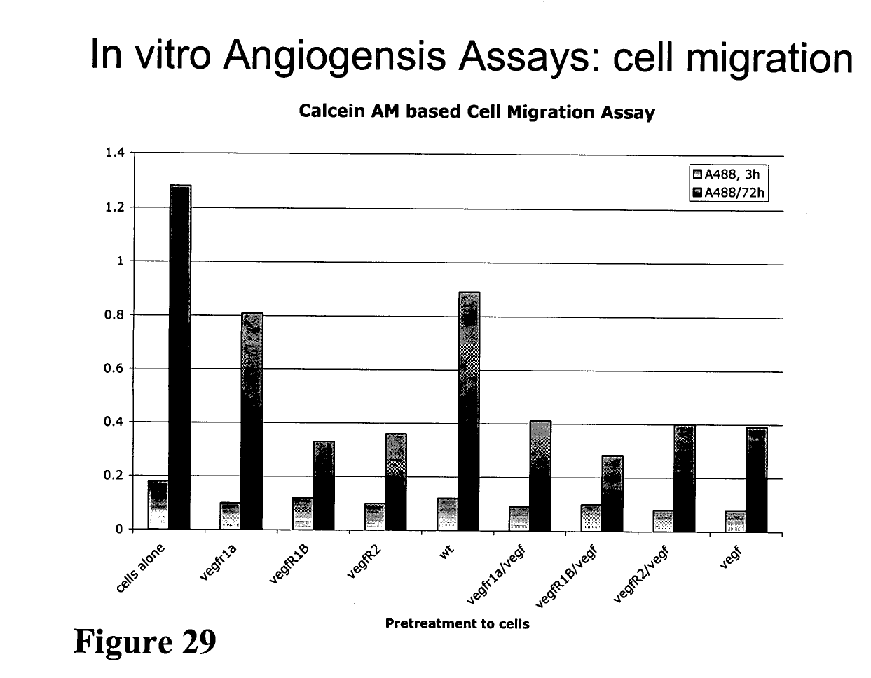

Vascular permeability/endothelial cell migration assay. Migration (chemotaxis) of calcein AM labeled HUVECS induced by VEGF was tested in the presence/absence of CPMV-Vegf chimeras or wt CPMV. VEGF stimulated the migration of cells across the filter by 30%. This effect was counteracted by the VegfR1 targeting chimeras but not by the wt CPMV (FIG. 29).

FIG. 29 shows cell migration in in vitro angiogensis assays. The migration ofcalcien AM labeled HUVECS stimulated by VEGF were tested in the presence or absence of CPMV VEGF R1 and R2 chimeras. The number of endothelial cells that transmigrated into the wells was quantified by measuring the intensity of fluorescence for calcein. (488 nm).

### Example 40

CPMV Chimeras Homing to Tumor Vasculature/Endothelial Cells in Animal Models

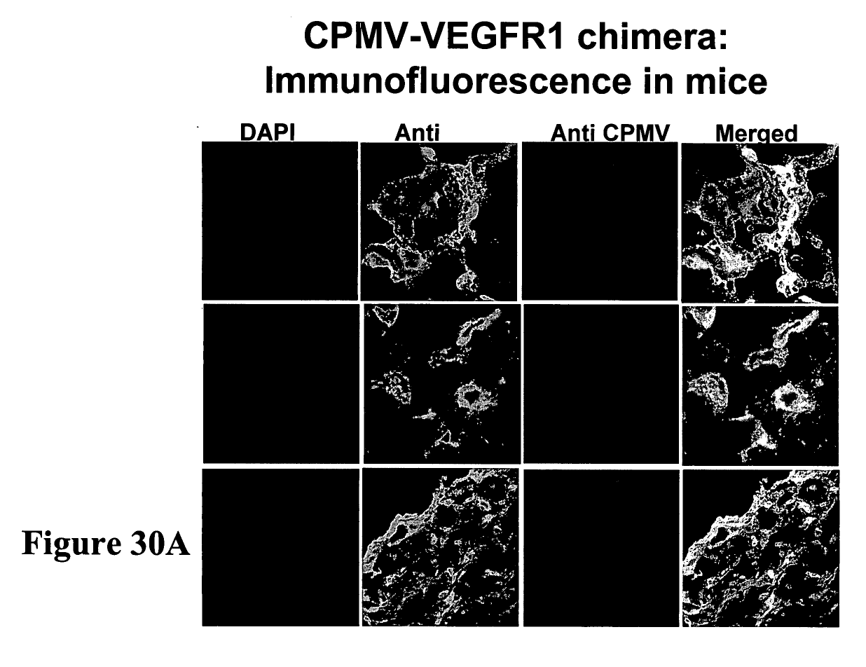

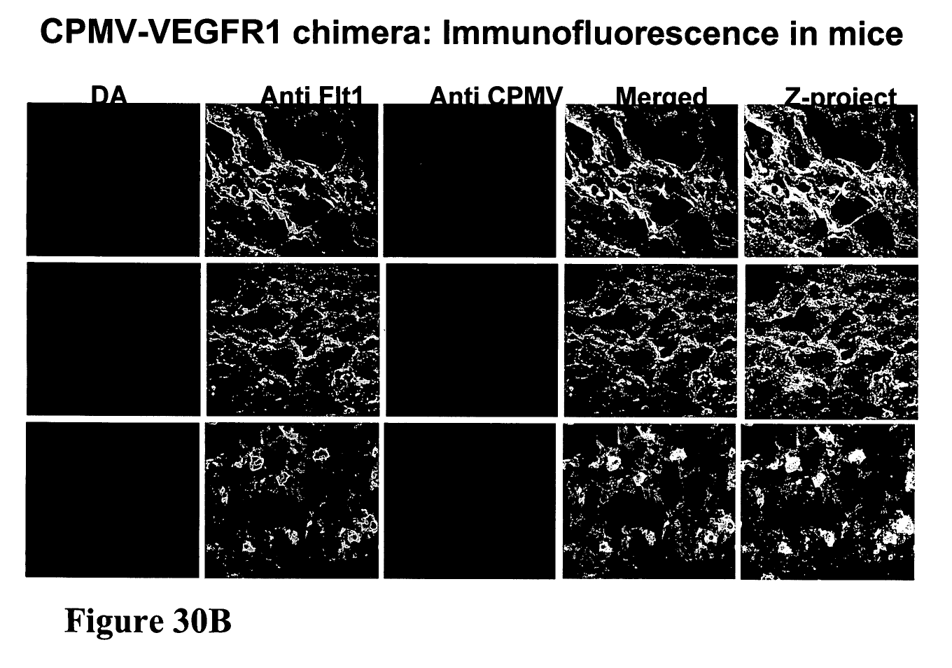

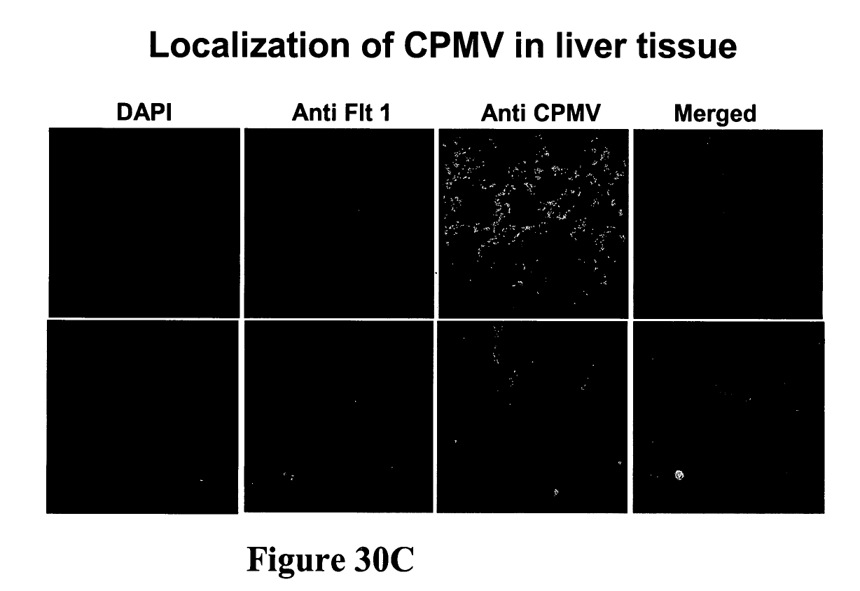

Based on our results in tissue culture, we extended our tests in SCID mice that had been injected with 10⁶ MCF7 or HT29 cells. Once the tumor were about 3-4 mm in size, the mice were injected with the VEGFR1-CPMV chimera in their tail vein. After 1 h the mice were sacrificed and the tumor tissue was harvested. Cryo-microtome sections of the tumor tissue were further evaluated by immuno-fluorescence using anti CPMV and anti Flt-1 receptor antibody. Excellent co-localization of the virus to the receptors on tumor tissue was observed suggesting that the virus particles were able to selectively home to tumor cells. (FIGS. 30A, 30B) There was some amount of virus accumulation in liver as well (FIG. 30C).

FIGS. 30A, 30B, and 30C show immunofluorescence of CPMV-VEGFR1 chimera in mice. FIG. 30A shows colocalization of CPMV with the VEGFR1 binding peptide (red, 595 nm) to the Flt-1/VEGFR1 receptor (green, 480 nm) in tumor tissues harvested from SCID mice. The mice were injected with the virus in the tail vein and sacrificed after 1 hour post injection. Tumor tissues were harvested, sectioned and probed with anti CPMV and anti Flt-1 antibodies using the confocal microscope. (Breast carcinoma cells were used to implant the tumor in mice) FIG. 30B shows immunofluorescence of CPMV-VEGFR1 chimera in mice implanted with colon cancer cells. FIG. 30C shows localization of CPMV in liver tissue.

Experiments are currently underway to determine the efficacy of the double mutant chimeras that carry DOX in addition to the VEGFR1homing peptide presented on their capsid to cause a regression in tumor growth and metastasis as well as to look for inhibition of angiogenesis in mice.

This study has demonstrated the ability to load, quantitative and selectively deliver Doxorubicin to tumor cells. The results illustrate the potential of virus particles to serve as versatile targeting and drug delivery systems.

### Example 41

Materials and Methods

Generation of FLT-1 and KDR targeting chimeras. Site-directed mutagenesis of pCP2 (coding for RNA2 of CPMV) to generate VEGFR1-CPMV, and VEGFR2-CPMV mutants was carried out based on established protocols (Burton et al., 1997). Synthetic oligonucleotides corresponding to the vegf R1 binding peptide (WHSDMEWWYLLG) or the R2 receptor (ATWLPPR VCDWEYWCG) were designed for site-specific insertional mutagenesis at the EF loop of the large subunit. An et al., Int J Cancer 111: 165-73, 2004. The peptide was inserted between residues 98-99 of the large subunit at the HpaI/KpnI site in the pCP2 clone. The clones were verified by sequencing and inoculated on plants with pCP1. The plants were grown for three weeks before harvesting. The purified virus was resuspended in 0.1M-phosphate buffer, 250 mm NaCl (pH7.5), 10 mM TCEP.

Binding and competition assays using ELISA. For the binding assay, 96 well plates were coated with the anti Flt1/anti KDR antibody for 1 h after which the plates were washed and the VEGF₁₆₅ receptor was added to the wells. To the immobilized receptor, the virus chimeras were added and incubated at 37° C. for 2 h. The plates were subsequently washed and the sample wells were incubated with anti CPMV antibody for 1 h. Finally the plates were washed three times with PBS containing 0.2% Tween 20 and the virus was detected using HRP conjugated secondary anti rabbit antibody. The color development reagent used was TMB and the plates were read at 480 nm using the optiplex microplate fluorescence reader (molecular Devices).

For the competition experiments, VEGF₁₆₅ was added to the virus sample before incubating with the immobilized Flt 1/KDR receptor.

Immunofluorescence. Confocal microscopy was used to detect the presence of the bound virus. HUVECs and MDA MB231 cells were seeded at a density of 2×10⁴ cells/well on coverslips in a 12 well culture plate and allowed to grow overnight at 37° C., 5% CO₂. Different targeting chimeras were added to the cells for 1 h after which the media was aspirated, the cells ere washed 3× in PBS and fixed in 4% paraformaldehyde solution for 10 minutes at 37° C. Dcells were subsequently washed 3× in PBS and blocked in 10% goat serum (for cells) or superblock (for histology, tissue sections) for 1 h at 37° C. The cells were incubated with primary antibody (1:5000 dilution, polyclonal rabbit, anti CPMV, ATCC) for 1 h. After 3 washes in PBS, the cells were exposed to secondary FITC conjugated anti rabbit antibody for 1 h. The cells were finally washed 3× in PBS and mounted in Prolong antifade medium and observed under the microscope (BioRad, 2100 Radiance). For histology analysis, anti human Flt1 antibody (Abcam) was used as the primary antibody that was detected using anti mouse Rhodamine conjugated secondary antibody. Both the secondary antibodies were purchased from Molecular Probes.

Measure in vitro angiogenesis by endothelial cell proliferation assay. HUVECs were plated in 96 well plates at 10⁴ cells/well in 200 ul EGM2 complete medium. Cells were incubated for 24 h at 37° C. in 5% CO₂. cells were then washed once in PBS and incubated in serum free, glucose free medium for 24 h to suppress cell growth. Further, the cells were exposed to various treatments like 10% FBS or VEGF165 in the presence or absence of the targeting CPMV chimeras. Wild type CPMV was used as a control. After 48 h incubation, 1 μl of CFDA was added to the wells and the plated were incubated for additional 24 h. The cells were then placed on ice, washed in PBS 3× and finally fixed in cold 10% TCA for 10 minutes. After washing, the cells were lysed and the CFDA incorporation was read at 488 nm.

Endothelial cell migration assay. Migration of calcein AM labeled cells (5 μM, 30 min, at 37° C.) was tested in the presence or absence of the CPMV chimeras using cell migration chambers Briefly, the lower wells were loaded with buffers containing various chemo-attractants. The framed filter membrane was positioned on the top and 50,000 calcein labeled HUVECs treated with/without the targeting chimeras were added to the top well above the membrane. The assembly was incubated for 2 h at 37° C. and the number of cells that transmigrated to the bottom wells was quantified by measuring the intensity of fluorescence at 488 nm.

Animal model experiments. 1×10⁶ cells from the breast cancer cell line, MDA MB 231 and the colon cancer cells HT29 were implanted into the 6-8 week old immuno-compromised Balb/c nude mice subcutaneously. Tumor growth was monitored and once the tumors reached an acceptable size, the mice were injected with the targeting CPMV chimeras (0.1 ml, 20 ug) intravenously into the tail vein. After one hour, the mice were sacrificed and parts of the tumor tissue and some other organs were harvested and frozen for histological and bio-distribution studies. The tumor were sectioned with a cryo-microtome and analyzed for the presence of virus using anti CPMV antibody. For co-localization studies, anti Flt 1 antibody was used. Secondary antibodies were either FITC-or rhodamine conjugated anti rabbit or anti mouse antibodies and obtained from Molecular Probes. Appropriate anti mouse IgG Isotype controls were used to determine the background fluorescence. All immuno-fluorescence/histology experiments were analyzed by confocal microscope (BioRad 2100 radiance).

### Example 42

Polyvalently Displayed Carbohydrates on Viral Nanoparticles

The strength and selectivity of binding interactions between polyvalently displayed carbohydrates and target cells are likely to depend on the number and flexibility of the arrayed sugars. In one aspect of the invention, a virion can be covered as densely as possible with carbohydrate groups. Increasing the degree of virus coverage requires the reactive polymer end group to be compatible with polymer synthesis and/or elaboration and yet reactive enough to accomplish a demanding subsequent connection to the virus coat protein—a union of two large molecules present in low concentrations.

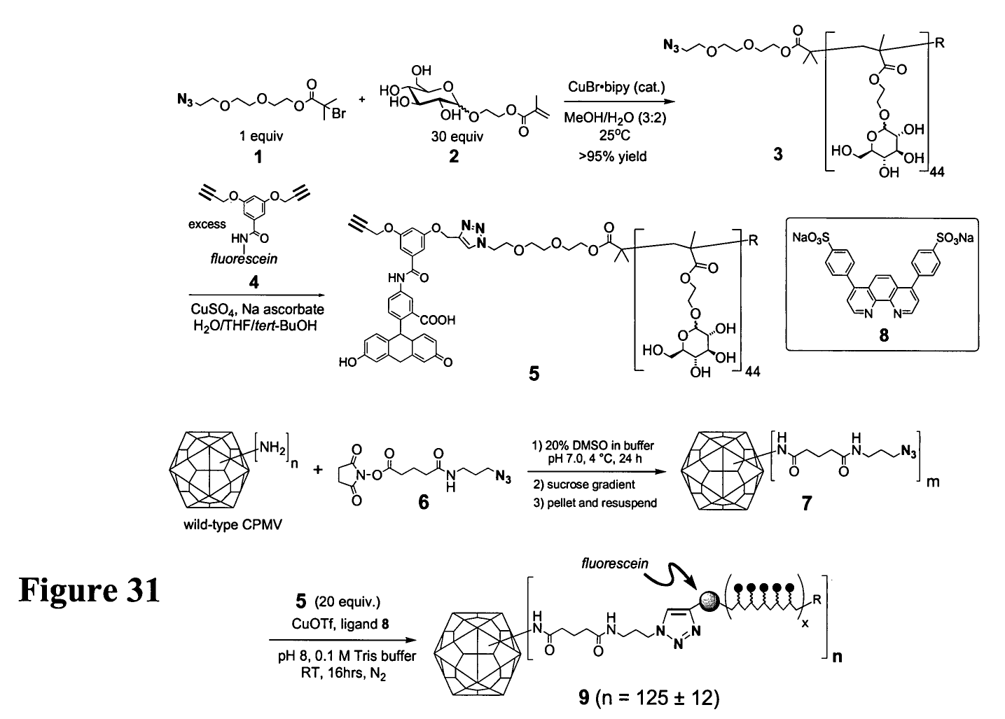

The side-chain neoglycopolymer 3 was prepared by atom transfer radical polymerization (ATRP) of methacryloxyethyl glucoside (2) using azide-containing initiator 1 (FIG. 31). Gaynor et al., Macromolecules 31: 5951, 1998; Narain and Armes, Macromolecules 36: 4675, 2003. The presence of the azide chain end in the polymer was confirmed by colorimetric test and by the presence of the characteristic peak at 2100 cm-1 in the infrared spectrum. Punna and Finn, Synlett, 99, 2004. GPC analysis established the clean nature of the material and an average molecular weight (Mn) of 13,000 with polydispersity of 1.3, consistent with the initiator:monomer ratio used and with expectations for ATRP of acrylates in water. Narain and Armes, Macromolecules 36, 4675, 2003; Matyjaszewski, Chem. Eur. J. 5: 3095, 1999; Coessens and Matyjaszewski, J. Macromol. Sci.-Pure Appl. Chem. A36: 667, 1999; Li et al., J. Polym. Sci. A: Polym. Chem. 38: 4519, 2000.

Azide-terminated polymer 3 was elaborated to the alkyne-terminated form 5 by reaction with fluorescein dialkyne 4. FIG. 26. The excess dye was removed by filtration and the polymer products were further purified by size-exclusion chromatography (Sephadex G-15). The complete conversion of the azide to the alkyne end group was confirmed by the observation of a negative colorimetric test and by the disappearance of the azide IR resonance (the corresponding alkyne resonance is much less intense and therefore not visible). The chromophore thus installed serves as a spectroscopic reporter for subsequent manipulations. The dimeric polymer, formed as a minor byproduct from the reaction of two molecules of 3 and one of 4, was not separated from 5 as it cannot participate in bioconjugation.

Cow pea mosaic virus (CPMV) was derivatized with N-hydroxysuccinimide 6 (NHS) to install azide groups at lysine side chains of the coat protein. FIG. 1. NHS esters have been previously established to acylate lysine residues over the external surface of the capsid, with loadings controlled by overall concentration, pH, and reaction time. Wang et al., Chem. Biol. 9: 805, 2002. In this case, conditions were employed which result in the derivatization of a substantial fraction of the approximately 240 solvent-accessible lysine side chains (m=approximately 150 in FIG. 1). The resulting azide-labeled virus (7) was then condensed with 20 equivalents of polymer-alkyne 5 in the presence of copper(I) triflate and sulfonated bathophenanthroline ligand 8 under inert atmosphere to produce the glycopolymer-virus conjugate 9 in excellent yield after purification by sucrose-gradient sedimentation to remove unattached polymer. Lewis et al., J. Am. Chem. Soc. 126: 9152, 2004. By virtue of the calibrated dye absorbance, the number of covalently bound polymer chains was found to be 125±12 per particle, representing the addition of approximately 1.6 million daltons of mass to the 5.6 million Da virion. This procedure, the general application of which will be described elsewhere, is far more efficient than the previous Cu(I)-mediated method, which required 100 equivalents of 5 with respect to azide to achieve similar results. Wang et al., J. Am. Chem. Soc. 125: 3192, 2003.

Covalent labeling of the vast majority of CPMV protein subunits with glycopolymer was confirmed by denaturing gel electrophoresis. The intact nature of the particle assembly and its larger size was verified by size-exclusion FPLC as well as transmission electron microscopy (TEM). TEM images revealed the virus conjugates to be more rounded in shape, to take on uranyl acetate stain differently, and to be 12-15% larger in diameter than the wild-type particle. The hydrodynamic radius and molecular weight of 9 were found by multi-angle dynamic light scattering (DLS) to be dramatically larger as well: 30.3±3.4 nm and 1.4±0.4×10⁷ Da, compared to 13.4±1.3 nm and 6.1±0.3×10⁶ Da for wild-type CPMV. That both radius and molecular weight values are substantially greater than expected reflects the uncertainties of calibration and interpretation of light scattering data for these unique polymer-virus hybrid species.

### Example 43

Specific Targeting of CPMV Nanoparticles to Tumor Cells

The ability to target tumors and deliver therapeutics to specific locations in the body is a primary goal in cancer medicine. Tumor-targeting strategies include the use of various types of nanoparticles such as liposomes, iron oxide nanoparticles, silica-gold nanoshells and highly branched macromolecules called dendrimers. Lee, et al. J. Bio. Chem. 269: 3198-3204, 1994; Sonvico et al. Bioconj. Chem. 16: 1181-8, 2005; Hirsch et al. Proc Natl Acad Sci USA 100: 13549-54, 2003; Quintana, et al. J. Pharm Res. 19: 1310-6, 2002; Choi et al. J. Cell Cycle 4(5): 669-671, 2005. Antibodies or other types of ligands capable of targeting tumors are typically attached to the exterior surface and in many cases drugs or contrast agents can also be encapsulated inside the particles for cell killing or tumor imaging. Wang, Journal of Controlled Release 53: 39-48, 1998.

One of the best-known tumor markers is the folate receptor (FR) that is up-regulated or over-expressed on a variety of human tumors, including cancers of the ovary, kidney, uterus, testis, brain, lung and myelocytic blood cells. Leamon, et al. Biochem J 291: 855-860, 1993; Reddy, Crit Rev Ther Drug Carrier Syst. 15: 587-627, 1998; Reddy, Journal of Controlled Release 64: 27-37, 2000; Wang, Journal of Controlled Release 53: 39-48, 1998; Lu, Adv. Drug Deliv. Reviews 54: 675-693, 2002. The 0.44-kDa vitamin folic acid (FA) plays an essential role in human growth and development, in particular cell division and DNA synthesis. Binding of FA to FR initiates receptor-mediated endocytosis, although the pathway used to reach the endosomal compartment is still controversial. Rijnboutt, et al. J Cell Biol 132(1-2): 35-47, 1996; Birn, et al. Am J Physiol 264(2 Pt 1): C302-10, 1993; Maxfield, et al. Nat Rev Mol Cell Biol 5(2): 121-32, 2004. Because the demand for FA increases when human cell growth is very active, such as during cancer cell proliferation, FA can be used to specifically target several types of tumor cells.

Derivatization of liposomes with FA has shown great potential in tumor targeting. Lee, The Journal of Biological Chemistry 269: 3198-3204, 1994. Recently, the importance of the presence of a PEG spacer between the FA and the liposome has been suggested to facilitate binding of the conjugated FA to FR. Gabizon et al. Bioconj. Chem. 10: 289-298, 1999; Gabizon, et al. Adv. Drug Deliv. Reviews 56: 1177-1192, 2004; Stephenson et al. Methods Enzymol. 387: 33-50, 2004. FA-coated gadolinium nanoparticles have also shown increased uptake in tumor, as well as the possibility for neutron capture therapy agents. Oyewumi et al. Journal of Controlled Release 95: 613-626, 2004. Poly-amidoamine dendrimers conjugated to FA were shown to be effective in cancer targeting and tumor cell uptake of methotrexate. Quintana, et al. J. Pharm Res. 19: 1310-6, 2002. Further, recent work by Choi and Baker employed FA as the targeting agent on a dendrimer nanocluster. Choi et al. J. Cell Cycle 4(5): 669-671, 2005.

In this study the Cowpea Mosaic Virus (CPMV) was used as a potential nanoparticles candidate for tumor targeting. CPMV is a 31 nm, icosahedral plant virus that grows in the common cowpea plant (Vigna unguiculata). Lomonossoff, et al. Program. Biophys. Molec. Biol. 55: 107-137, 1991. CPMV has a bipartite positive-sense, single-stranded RNA genome with each RNA molecule (designated RNA1 and RNA2) encapsidated in a separate particle. Both RNA molecules are required for infection of plants, and infectious cDNA clones are available in the laboratory. Lomonossoff, et al. Proceedings of the Second AFRC Protein Engineering Conference, Goodenough, P., Ed. CPL Press: 1993. CPMV capsids are composed of 60 copies each of a large (L; 42 kDa) and small (S; 24 kDa) capsid protein to form a 31 nm-diameter pseudo T=3 icosahedral particle. CPMV grows to very high yields in infected plants and the purification is straightforward and inexpensive In addition, CPMV is non-pathogenic for humans, and the products derived from plant virus culture are not contaminated with animal cells or viruses. Brennan, et al. Mol. Biotechnol. 17: 15-26, 2001; Johnson, et al. Annu Rev Phytopathol 35: 67-86, 1997. CPMV particles are highly stable to temperature, pH and a variety of organic solvents such as DMSO. Lomonossoff, et al. Program. Biophys. Molec. Biol. 55: 107-137, 1991. Chemical modification of CPMV surface lysine residue using fluorescent dye-labeled N-hydroxysuccinimide (NHS) esters has been extensively characterized. Wang, et al. Chem. Biol. 9: 805-11, 2002; Wang, et al. Angew. Chem. Int. Ed. 41: 459-462, 2002. Most recently CPMV particles have shown to be stable during azide-alkyne cycloaddition (“click” reaction), dramatically increasing the variety of ligands that may be conjugated to the CPMV surface. Sen Gupta, et al. Bioconjug Chem 16(6): 1572-9, 2005.

In vivo studies using CPMV have shown great potential for these particles for use in imaging of both normal and tumor vasculature. Lewis, et al. Nature Medicine (submitted), 2006. In that study wild type CPMV associated preferentially with the endothelium of the vascular system and was taken up into endothelial cells, while PEG-coated CPMV did not. These studies demonstrated the utility of CPMV in vivo but underscored the need for cell-specific targeting.

This study demonstrated that folate could be used to direct CPMV specifically to tumor cells in vitro and the pattern of CPMV binding or uptake could be affected by retargeting the virus to the folate receptor. Azide-alkyne cycloaddition was used to conjugate FA, or PEG-conjugated FA, to the particle surface. The ability of FA-modified CPMVs to specifically target tumor cells while eliminating background specificity was addressed by in vitro assays and flow cytometry studies.

### Example 44

Materials and Methods

All reagents, unless otherwise specified, were purchased from commercial suppliers and used without further purification. Bi-functional N₃-PEG-NH₂ (MW: 570, PEG 500, PDI=1) was obtained from Polypure Inc. Copper (I) triflate was prepared according to literature procedures.

Propagation of CPMV in Plants. The primary leaves of Kentucky cowpea plants (Vigna Unguiculata) were mechanically inoculated as 10-day old seedlings, bearing two primary leaves and with secondary leaves just beginning to show. Virus stocks were initiated from pCP1 and pCP2 plasmid cDNAs encoding full-length copies of the two RNA moieties of CPMV, RNA-1 and RNA-2, respectively. Carborandum was first dusted onto the leaves to aid in the wounding process. At approximately 3 weeks post inoculation, the symptomatic leaves were harvested, weighed and frozen at −70° C. until ready to purify the virus. The virus was purified from the infected leaves by a method as described. Dessens, J. T.; Lomonossoff, G. P. J. Gen. Virol., 74: 889-892, 1993.

Synthesis and Characterization of CPMV-PEG-FA Virus: Synthesis of N₃-PEG-FA. FA-NHS ester was prepared from FA according to literature procedures. Lee, et al. J. Bio. Chem. 269: 3198-3204, 1994. FA-NHS (100 mg, 0.19 mmol) was dissolved in anhydrous DMSO (10 ml). N₃-PEG-NH₂ (50 mg, 0.09 mmol) was added to the solution of FA-NHS and agitated at RT for 20 hours. DMSO was removed under high vacuum at RT. The residue was sonicated into a fine powder, transferred to a fritted disk, and washed thoroughly with diethyl ether, CH₂Cl₂, THF. The residual dark orange solid was dried and extracted with water until the aqueous extract was colorless. The aqueous fractions were combined and evaporated under vacuum to yield N₃-PEG-FA as viscous orange oil (60 mg, 0.06 mmol, 66%). ¹H NMR (200 MHz, D₂O): δ 8.59 (s, 1H), 7.52 (d, 2H), 6.64 (d, 2H), 4.48 (s, 1H), 4.15 (m, 2H), 3.58-3.35 (m, 40), 2.35-2.20 (m, 12H)

Preparation of CPMV-Alkyne. Compound X (50 mg, 0.20 mmol) was dissolved in DMSO (1 ml) and added to a solution of wt-CPMV (5 ml, 7 mg/ml, 0.1 M phosphate buffer, pH 7.0). The mixture was agitated at RT for 15 hours and purified by sucrose gradient fractionation (10-40% sucrose in 0.1 M pH 7.0 phosphate buffer, Beckman SW-28 Ti rotor, 28000 rpm, 3 hours). The intact virus was collected as a pale white band under intense illumination on a gradient fraction collector and subjected to ultracentrifugation (Beckman 50.2 Ti rotor, 42000 rpm, 3 hours) to form a colorless pellet. The solution was decanted and the colorless pellet was dissolved under N₂ with sufficient buffer (Tris Cl, 0.1 M, pH 8.0) to obtain a concentration of 7.3 mg/ml.

Conjugation of N₃-PEG-FA to CPMV-Alkyne. The following reaction protocol was carried out in an inert atmosphere (N₂) glove box with O₂ level kept below 6 ppm until the final gel filtration step. For each of the three N₃-PEG-FA loading levels, a 2 mL Eppendorf centrifuge tube was charged with CPMV-alkyne (7.3 mg/ml, 110 μl) and buffer (Tris Cl, 0.1 M, pH 8.0, 260, 250, 220 μl respectively). A degassed aqueous solution of N₃-PEG-FA (25 mM, 10, 20, 50 uL respectively) was added to the virus solution and mixed by gentle agitation. A solution of copper (I) triflate (100 mM, CH₃CN, 50 μl) was combined with a solution of sulfonated bathophenanthroline (100 mM, Tris Cl, 0.1 M, pH 8.0, 150 μl) to form a catalyst mixture. An aliquot of the catalyst mixture (16 μl) was added to the tube containing the virus. The reaction mixture was immediately placed on a rotisserie for continuous agitation, and kept under N₂ at RT for 15 hours. The product was purified by three passages through size exclusion gel filtration columns (BioRad, p-100) which removed all residual catalyst and excess N₃-PEG-FA. The integrity of the virus was verified by analytical size exclusion chromatography (Superose 6) and TEM. Concentration of the virus was determined by UV-Vis spectroscopy, by measuring the absorbance at 260 nm; virus at 0.1 mg/ml gives a standard absorbance of 0.8. The average molecular weight of the CPMV virion is 5.6×10⁶.

Conjugation of N₃-PEG-NH₂ and N₃-PEG-Fluorescein to CPMV-Alkyne. Conjugation of N₃-PEG-NH₂ and N₃-PEG-Fluorescein to CPMV-Alkyne was performed following the procedures described above for the conjugation of N₃-PEG-FA. The quantities of reagents were: CPMV-alkyne (7.3 mg/mL, 110 μl), buffer (Tris Cl, 0.1 M, pH 8.0, 250 μl), N₃-PEG-NH₂ or N₃-PEG-Fluorescein (25 mM, 20 μl).

Cell Culture and Binding Studies. HeLa cells and KB cells, a human nasopharyngeal epidermal carcinoma, were grown continuously as a monolayer using folate-free RPMI1640 medium (Gibco, Invitrogen, Carlsbad Calif.) containing 10% heat-inactivated fetal bovine serum (FBS), penicillin (50 units/ml), streptomycin (50 μg/ml), and 2 mM L-glutamine at 37° C. in a 5% CO₂/95% air humidified atmosphere. Saikawa, et al. Biochemistry 34: 9951-9961, 1995. The concentration of folic acid was 5-6 nM in folate-free medium containing serum, therefore close to the natural physiologic conditions. Gabizon et al. Bioconj. Chem. 10: 289-298, 1999; Antony, Blood 79: 2807-2820, 1992. On the day before each experiment, the medium was replaced with folate-free RPMI 1640 containing all the supplements mentioned above, except 10% FBS.

Measurement of Virus Binding to HeLa and KB cells using Flow Cytometry. HeLa and KB cells, grown overnight in folate-depleted medium were trypsinazed, counted and 100 μl of cells were plated in a 96-well V-bottom shaped plate at a concentration of 5×10⁶ cells/ml. 10 μg of different virus preparations were added to each well and the cells were incubated on ice at 4° C. for 1 hour. Following incubation, cells were washed 4 times using ice cold PBS buffer, containing 1 mM EDTA and 25 mM HEPES pH 7.5, at 1600 rpm, 6 minutes at 4° C. Rabbit anti-CPMV primary antibody was then added to the cells in a 100 μl volume, and the cells were incubated on ice at 4° C. for 30 minutes. Cells were then washed as mentioned above. Goat anti-Rabbit IgG AlexaFluor 488 conjugated antibody (Invitrogen, Carlsbad Calif.) was then added to the cells in a 100 μl volume, and the cells were incubated on ice at 4° C. for 30 minutes in the dark. Following incubation, cells were then washed as mentioned above. Finally cells were fixed using 2% formaldehyde in PBS buffer, containing 1 mM EDTA and 25 mM HEPES pH 7.5. The samples were then analyzed using a FACS Calibur machine (BD Biosciences, Franklin Lakes, N.J.). Approximately 50,000 events were collected for each sample and data was analyzed by FlowJo software (Tree Star, Inc).

Cellular Uptake of the Folate-Conjugated Virus in HeLa and KB Cells. Cells were seeded in a 12-well plate containing 12 mm sterile glass cover slids at 1×10⁵ cells/well and grown for 48 hours as previously described. On the day of the experiment, cells were washed once with folate-depleted medium, 10 μg of different virus preparations were added to each well and the cells were incubated at 37° C. in a 5% CO₂/95% air humidified atmosphere for 2 hours. Cells were then washed 4 times using folate-depleted medium to remove unbound virus, on a rocker at room temperature for 5 minutes. Cells were then fixed using 4% para-formaldehyde in PBS for 20 minutes. After 4 washes using PBS, cells were permeabilized using 0.1% Triton X-100 in PBS, for 15 minutes. Non-specific binding was blocked by incubating the cells in 5% goat serum in PBS, for 1 hour. Rabbit anti CPMV antibody was added to the cells in 1% goat serum, 0.1% Triton X-100 in PBS, and cells were incubated at room temperature for 45 minutes with gently agitation. Unbound antibody was then removed by washing 4 times with PBS. Goat anti-rabbit IgG AlexaFluor 488 conjugated antibody (Invitrogen) was added in 1% goat serum in PBS, and cells were incubated for 35 minutes at room temperature with gently agitation. During the last five minutes of secondary antibody incubation, cell nuclei were stained by adding 100 μl of Hoechst 33258 (1:1000 dilution in water). Cells were then washed 4 times using PBS and cover slips covered with cells were mounted on slides using Vecta Shield mounting medium (Vector Laboratories). Cells were imaged using a Nikon Eclipse TS 100 microscope, with a 100×-oil objective.

### Example 45

Synthesis of PEG-FA and Attachment to CPMV

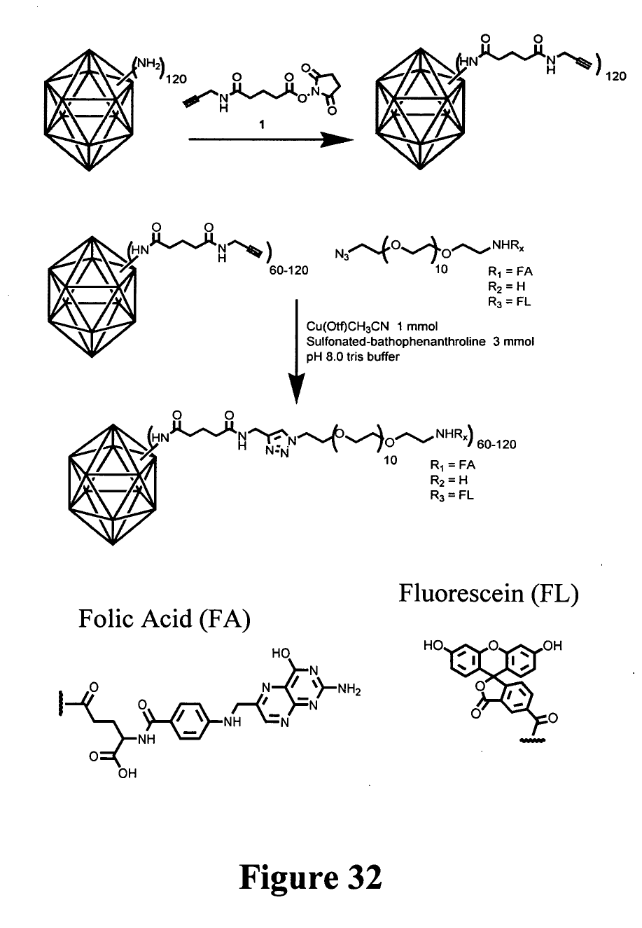

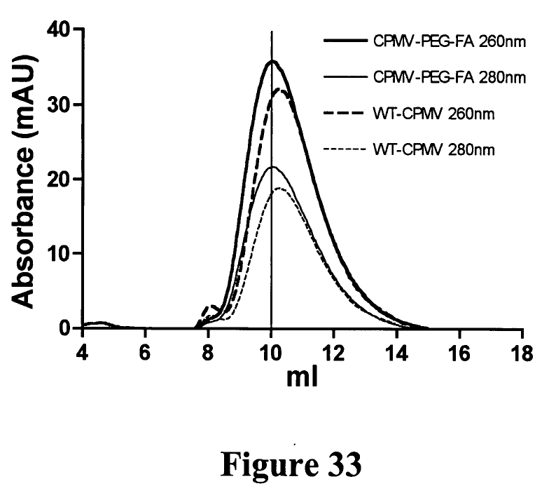

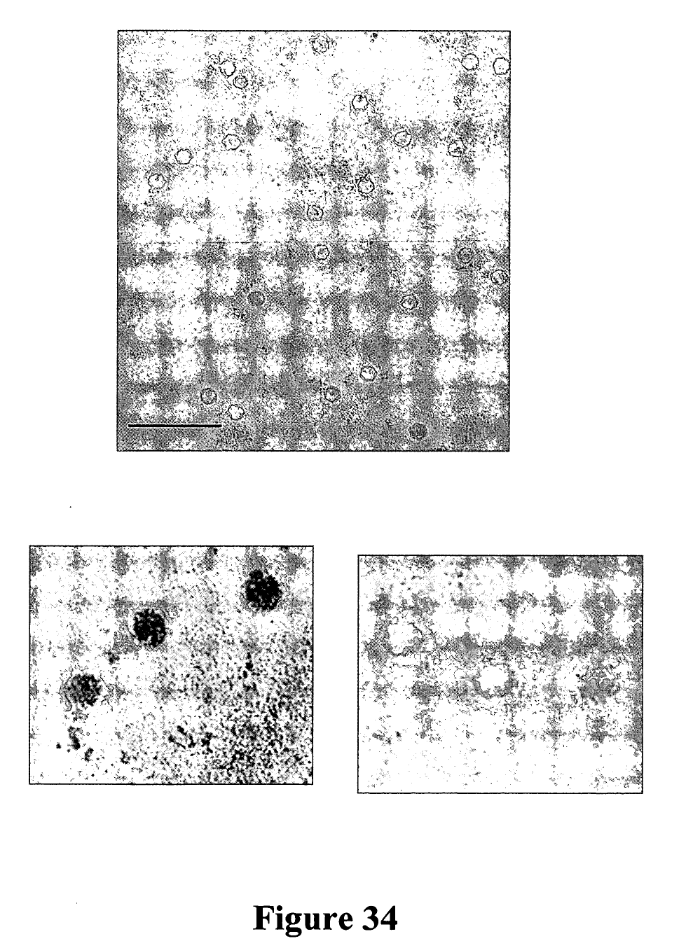

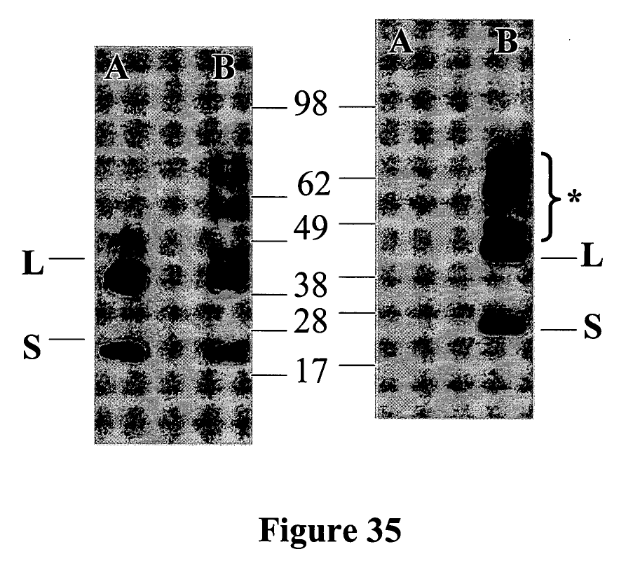

Receptors for the vitamin folic acid are upregulated or over-expressed on a broad variety of tumor types. Lu, Adv. Drug Deliv. Reviews 54: 675-693, 2002. The polyvalent display of folic acid on such scaffolds as liposomes and iron oxide nanoparticles has been utilized for cancer cell targeting. Sonvico et al. Bioconj. Chem. 16: 1181-8, 2005; Lee, The Journal of Biological Chemistry 269: 3198-3204, 1994. In this study direct conjugation of FA-NHS ester to CPMV was first attempted. Although the chemical reaction was successful, even with extensive folate conjugation, both flow cytometry analysis and in vitro cell uptake studies did not show significant specific binding in comparison to control CPMV. Non-specific binding of CPMV to cells has already been demonstrated. Lewis, et al. Nature Medicine (submitted), 2006. In addition, studies have shown that the presence of a spacer is required for cellular recognition of PEG-conjugated nanoparticles. Lee, et al. J. Bio. Chem. 269: 3198-3204, 1994. Next a 500 Da PEG chain to function as a spacer between the virus surface and folic acid was introduced using azide-alkyne cycloadditon. FA-NHS was incubated with N₃-PEG-NH₂ at a molar ratio of 2:1 and agitated at RT for 20 hours. In the final purification step N₃-PEG-FA was evaporated under vacuum to yield a viscous orange oil. CPMV-alkyne was prepared by incubation of wt-CPMV with compound X (FIG. 32) for 15 hours at RT, yielding a colorless solution after the final purification step. CPMV-alkyne was incubated with N₃-PEG-FA, followed by purification by gel filtration to remove residual catalyst and excess N₃-PEG-FA. To quantitate the amount of N₃-PEG-FA conjugated to CPMV, a parallel reaction was performed, wherein N₃-PEG-fluorescein was reacted with CPMV-alkyne. From the absorbance of fluorescein measured by UV-Vis spectroscopy the loading was estimated to be 30-40 PEG-FA per virus particle (˜6 pmoles of folic acid/μg of CPMV). The integrity of the conjugated virus preparations was verified by analytical size exclusion chromatography (Superose 6), showing that the conjugated virus migrated through the column exhibiting a slightly larger size than unmodified CPMV, as expected (FIG. 33). Transmission electron microscopy (TEM) (FIG. 34) of negative-stained samples confirmed that the particles were intact in comparison to unmodified particles, and showed that the CPMV-PEG-FA conjugates existed as individual particles, with no evidence of aggregation. Finally, examination of the CPMV-PEG-FA and wt-CPMV products by Western immunoblotting showed a substantial apparent molecular weight increase of the virus coat protein subunits due to the presence of covalently-attached PEG, also confirming the successful conjugation of N₃-PEG-FA to CPMV-alkyne (FIG. 35).

FIG. 34 shows TEM images of a purified preparation of folate-PEG CPMV virus showing intact particles. The samples were stained with 0.2% uranyl acetate, and the images were acquired with a Philips Tecnai (100 Kv) electron microscope. The bar represents 200 nm.

FIG. 35 shows Western blots of wild type CPMV (A) and CPMV-PEG-FA (B). Left panel, viruses were detected using Rabbit anti-CPMV antibody. Right panel, viruses were detected using Rabbit anti-folic acid antibody. L: large subunit, and S: small subunit. The (*) indicates PEGylated subunits.

### Example 46

FA-Mediated Binding of CPMV to Tumor Cells

The ability of CPMV-PEG-FA compared to unmodified CPMV or CPMV-PEG to bind to folate-receptor expressing tumor cell lines KB and HeLa was assessed by flow cytometry. KB cells in particular are known to over-express FR at a level of approximately 2.8×10⁵ receptors per cell. Saul, et al. Journal of Controlled Release 92: 49-67, 2003. Virus binding and subsequent steps were carried out at 4° C. in order to minimize endocytosis. As expected, wt-CPMV showed significant non-specifig binding to the cell surface in both cells lines, while decoration of wt-CPMV with PEG greatly reduced this phenomenon.

By comparing the binding profile of CPMV-PEG to CPMV-PEG-FA, it was also demonstrated that specificity for the FR was achieved by using CPMV-PEG-FA, which showed high binding affinity for FR. In general, both cell lines showed increased binding of CPMV-PEG-FA compared to unmodified CPMV. The difference in binding between folate-conjugated CPMV compared to unmodified CPMV was ˜40 fold greater in KB cells than in HeLa cells.

### Example 47

Uptake of Folate-Conjugated Virus in HeLa and KB Cells

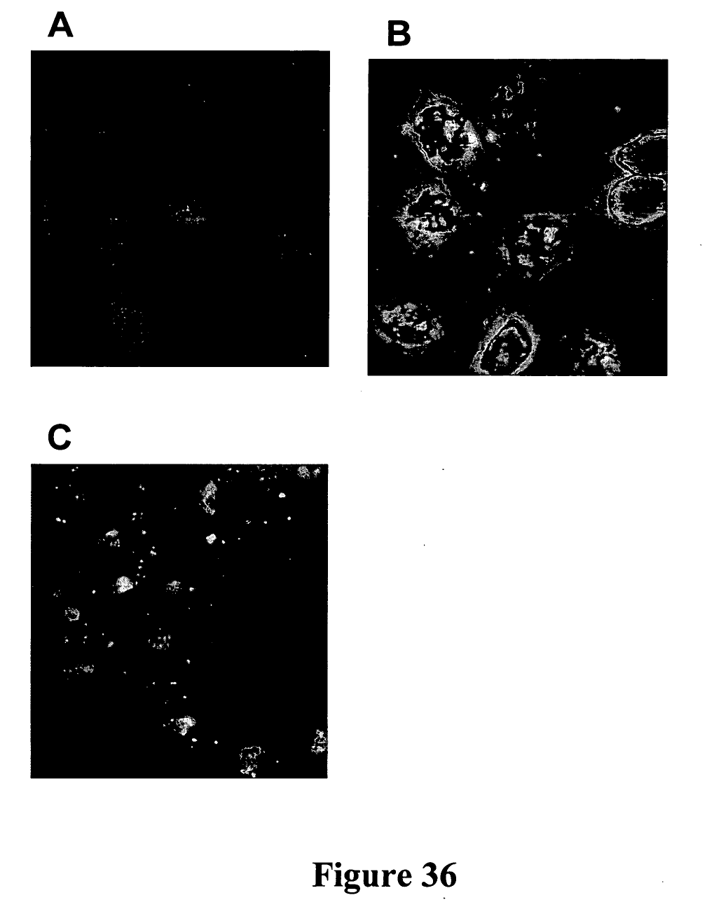

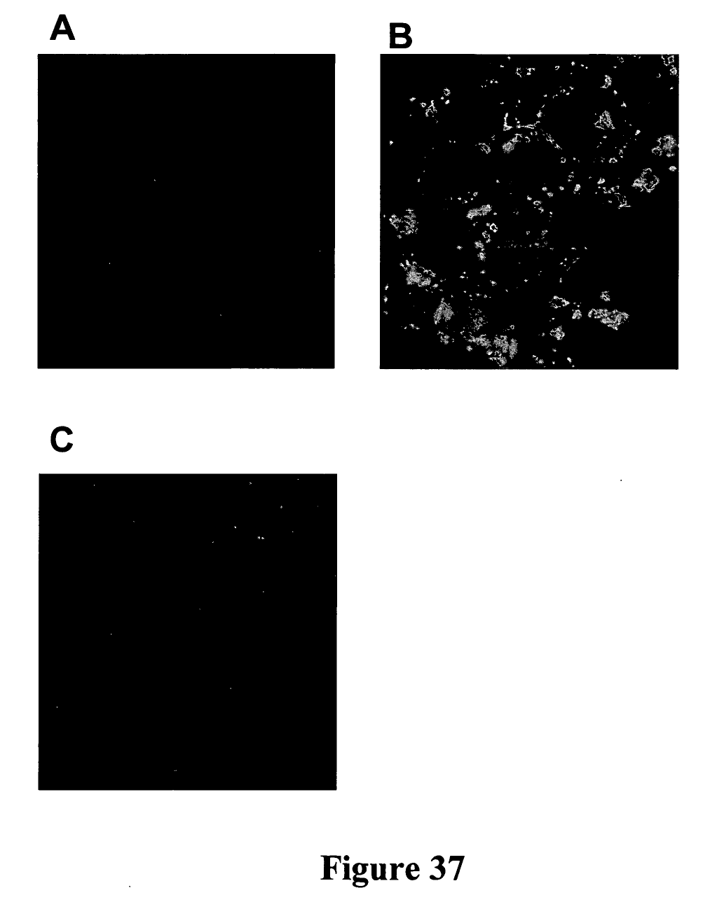

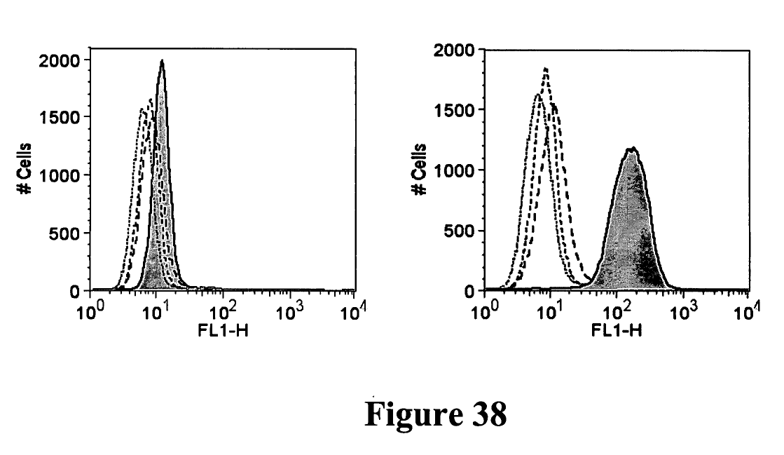

Cellular uptake of folate-conjugated compared to unmodified CPMV or PEGylated CPMV was analyzed by fluorescence microscopy. The different virus preparations were incubated with cells at 37° C. for 2 hours. Permeabilized cells were then visualized in the fluorescence microscope. Both HeLa and KB cell lines showed increased uptake of folate-conjugated viruses compared to unmodified CPMV (FIGS. 36 and 37). Non-specific binding and uptake of CPMV was similarly reduced when using CPMV-PEG. Uptake of CPMV-PEG-FA in HeLa cells appeared as a dispersed fluorescent layer, while KB cells showed a more punctuated distribution of the virus throughout the cells. This could suggest a different endocytic pathway of uptake and trafficking of the virus in the two cell lines. Also non-specific uptake of unmodified CPMV was higher in HeLa cells compared to KB cells, which correlates well with the data obtained by flow cytometry (FIG. 38).

FIG. 36 shows HeLa cell monolayers were incubated with CPMV-PEG (A), CPMV-PEG-FA (B) or WT-CPMV (C) for 2 hours at 37° C. and viewed by fluorescence microscopy with a 100×-oil objective.

FIG. 37 shows KB cell monolayers were incubated with CPMV-PEG (A), CPMV-PEG-FA (B) or WT-CPMV (C) for 2 hours at 37° C. and viewed by fluorescence microscopy with a 100×-oil objective.

FIG. 38 shows measurement of virus binding to KB (left panel) and HeLa (right panel) cells using Flow Cytometry. Points, short dashes, long dashes and filled area represent “Cells Only”, “CPMV-PEG”, “WT-CPMV” and “CPMV-PEG-FA” respectively.

### Example 48

Targeting of CPMV Nanoparticles Specifically to Tumor Cells

This study shows that direct chemical conjugation of a novel PEG-FA moiety to the CPMV surface by azide-alkyne cycloaddition was efficient and resulted in an estimated 30-40 molecules of FA per particle. Quantifying the binding and uptake of non-targeted and CPMV on tumor cells showed that there was significant background binding that could be attributed to the particle surface interacting with the cells. Masking the CPMV surface with PEG abrogated this binding, and the particles could then be redirected to the folic acid receptor via the conjugated FA, showing approximately 20-fold enhancement of binding compared to CPMV-PEG control. These studies indicate that CPMV nanoparticles can be effectively redirected by surface conjugation to ligands of interest, allowing specific uptake into tumors while avoiding nonspecific uptake into normal cells.

Direct conjugation of FA to CPMV did not produce the proper specificity for FR. The proximity of FA to the virus capsid is likely to result in steric impediment to receptor binding as it was previously demonstrated. Gabizon et al. Bioconj. Chem. 10: 289-298, 1999; Gabizon, et al. Adv. Drug Deliv. Reviews 56: 1177-1192, 2004; Stephenson et al. Methods Enzymol. 387: 33-50, 2004. The targeting virus was therefore prepared to contain both FA and a PEG spacer, in order to achieve the necessary flexibility for recognition by FR. Interestingly, it appears that by using a shorter arm PEG compare to previous studies binding to FR is still possible. Gabizon et al. Bioconj. Chem. 10: 289-298, 1999; Gabizon, et al. Adv. Drug Deliv. Reviews 56: 1177-1192, 2004; Lee, et al. Biochim Biophys Acta 1233(2): 134-44, 1995.

Previous studies have shown that CPMV can interact with a variety of cell types both in vitro and in vivo. Lewis, et al. Nature Medicine (submitted), 2006; Rae, et al. Virology 343(2): 224-35, 2005; Singh, et al. Drug Development Research In press, 2006. Based on the particle size and biochemical characteristics, CPMV seems likely to interact with M-cells in Peyer's patches when orally administered in mice, or seems to be taken up by macropinocytosis by antigen presenting cells. Further, when dye-labeled CPMV was injected in the tail vein of an adult mouse, it associated preferentially with the lumen periphery of the vascular endothelium, allowing for a clear resolution of vascular structures in various organs. Because of this natural specificity for several mammalian cell lines, including KB human nasopharyngeal carcinoma and HeLa cells, a study to identify this possible CPMV binding protein is being conducted. PEGylation is therefore necessary in order to achieve the necessary flexibility for binding to FR and also to block CPMV non-specific binding to cells. In fact, a recent study by Lewis et al. showed that coating CPMV with PEG completely inhibited the internalization by chick embryo endothelial cells in vivo, and greatly reduced uptake by spleen and liver reticuloendothelial system in adult mice. Lewis, et al. Nature Medicine (submitted), 2006 Also since CPMV is proteinaceous in composition, the use of a polymer coating such as PEG helps reducing the immune response in vivo. Raja, et al. Biomacromolecules 4(3): 472-6, 2003.

The results clearly show that specificity to the FR is achieved by displaying PEG-FA on the virus capsid, which in turn reroutes the natural binding property of CPMV to a specific cellular receptor. Because it is known that at high surface density the folate molecule can form dimers, trimers and even self-assembling tubular quartets, thus preventing FA to bind to FR, the reaction was controlled so that only 30 to 40 PEG-FA molecules were attached to the virus capsid. Federica Ciuchi, et al. J. Am. Chem. Soc. 116: 7064-7071, 1994; Reddy, et al. Gene Ther 9(22): 1542-50, 2002. The difference in binding to FR between HeLa and KB cells may be in part due to the number of FR present of each cell line, along with a difference in the natural binding affinities of CPMV for the two cell lines. This system can be improved by using a CPMV mutant where also cysteines are available for chemical modification. Then, it would be possible to display FA at the end of a longer maleamide-PEG arm, and use a shorter NHS-PEG to react with the naturally available lysines to block the virus non-specific interaction with the cell surface. In fact, knowledge of the virus capsid structures allows sophisticated engineering of the outer and inner capsid, and a rational design of the tumor-ligand orientation and stochiometry in order to maximize tumor recognition. This ability to control the distribution of ligands on the virus capsid together with ease of production and genetic manipulation, gives a great advantage over other nanoparticles, where the final product is not always uniform and homogeneous, and synthesis is demanding in terms of time and scale. An important property of CPMV is its potential for multivalent display of different ligands on the surface or in the interior of the capsid. Ligands can be chosen to achieve first tumor recognition and then delivery of a payload to specific targets inside the cells. The capsid can be genetically modified to express tumor-targeting peptides and contrast agents can be loaded inside the capsid for imaging purposes.

These results correlate well with work that has been on going to identify a possible CPMV binding protein in mammalian cell lines. Inactivating the infectivity of CPMV particles while retaining their other materials properties can be accomplished using UV irradiation.

All publications and patent applications cited in this specification are herein incorporated by reference in their entirety for all purposes as if each individual publication or patent application were specifically and individually indicated to be incorporated by reference for all purposes.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

What is claimed:

1. A method for targeting or imaging a tissue in a vertebrate subject comprising
administering to the vertebrate subject a plant viral particle comprising a plurality of targeting/imaging molecules covalently attached to the viral particle, and
delivering the targeting/imaging molecules on the viral particle to the tissue in the vertebrate subject.

2. The method of claim 1 wherein the plant viral particle further comprises:
a viral subunit comprising a plurality of sites for the covalent attachment of the plurality of targeting/imaging molecules, and
a plurality of viral subunits assembled into the viral particle displaying the plurality of targeting/imaging molecules on the viral particle.

3. The method of claim 1 wherein the plurality of targeting/imaging molecules are attached by chemical crosslink to the viral particle.

4. The method of claim 3 further comprising a plurality of lysine residues on the viral subunit covalently attached to the plurality of targeting/imaging molecules.

5. The method of claim 1 wherein the tissue is a tumor or organ in the vertebrate subject.

6. The method of claim 1, wherein the vertebrate subject is a mammalian subject or an avian subject.

7. The method of claim 3, wherein the plurality of targeting/imaging molecules are small molecules, metal complexes, polymer, carbohydrates, polypeptides, polynucleotides, or fluorescent chemical molecule.

8. The method of claim 7, wherein the plurality of targeting/imaging molecules are polyethylene glycol conjugated to the targeting/imaging molecule.

9. The method of claim 7, wherein the plurality of targeting/imaging molecules are transferrin, RGD-containing polypeptide, protective antigen of anthrax toxin, neuropeptide Y, glycopolymer, polyethylene glycol, or folic acid.

10. The method of claim 9, wherein the plurality of targeting/imaging molecules are polyethylene glycol conjugated to folic acid.

11. The method of claim 1 wherein the plurality of targeting/imaging molecules are encoded by an exogenous nucleotide sequence in a viral particle genome.

12. The method of claim 11 wherein the exogenous nucleotide sequence encodes siRNA, shRNA, or antisense RNA.

13. The method of claim 11 wherein the exogenous nucleotide sequence encodes a foreign polypeptide expressed as part of a coat protein of the viral particle.

14. The method of claim 13 wherein the exogenous nucleotide sequence encodes a foreign polypeptide expressed as part of a βE-αF loop, βB-βC loop, C′-C″ loop, or an N-terminus of the coat protein of the viral particle.

15. The method of claim 13 wherein the foreign polypeptide is a tumor antigen, a viral antigen, a bacterial antigen, or a parasite antigen.

16. The method of claim 1 wherein the plurality of targeting/imaging molecules are ligands binding to tumor cell surface receptors.

17. The method of claim 16 wherein the plurality of targeting/imaging molecules are ligands binding to VEGF-1 receptor, Flk-1/VEGF-2 receptor, LyP1 tumor target, lung endothelium, α5β1 integrin, or αvβ3 integrin.

18. The method of claim 16, wherein the plurality of targeting/imaging molecules are polyethylene glycol conjugated to the ligands.

19. The method of claim 1 wherein the plurality of targeting/imaging molecules induce a cell mediated immune response to a tumor cell, virus, bacteria, or parasite.

20. The method of claim 19 wherein the plurality of targeting/imaging molecules are tumor antigens, viral antigens, bacterial antigens, or parasite antigens.

21. The method of claim 13 wherein the plurality of targeting/imaging molecules are polypeptides binding a therapeutic or diagnostic agent.

22. The method of claim 21 wherein the plurality of targeting/imaging molecules are peptides binding doxorubicin, verapamil, vincristine, or vinblastine.

23. The method of claim 1 further comprising detecting the targeting/imaging molecules on the viral particles in the vasculature.

24. The method of claim 23, wherein the targeting/imaging molecule is a fluorescent molecule for fluorescent imaging, gadolinium chelate molecule for magnetic resonance imaging, PET contrast agent or CT contrast agent.

25. The method of claim 1 further comprising displaying the plurality of targeting/imaging molecules on the surface of the viral particle.

26. The method of claim 1 further comprising displaying the plurality of targeting/imaging molecules on the interior of the viral particle.

27. The method of claim 1, wherein the plant viral particle is a Comovirus, Tombusvirus, Sobemovirus, or Nepovirus.

28. The method of claim 1 wherein the plant viral particle is a comovirus.

29. The method of claim 1 wherein the comovirus is a cowpea mosaic virus.

30. The method of claim 16 wherein the viral particle targets or images a vascular endothelium in the vertebrate subject.

31. The method of claim 30, wherein the viral particle targets or images the vascular endothelium to distinguish veins from arteries.

32. The method of claim 30, wherein the viral particle targets or images a tumor vasculature.

33. The method of claim 32, wherein the plurality of targeting/imaging molecules are ligands binding to a receptor on the tumor vasculature.

34. The method of claim 33, wherein the plurality of targeting/imaging molecules are ligands binding to VEGF-1 receptor or Flk-1/VEGF-2 receptor.

35. The method of claim 33, wherein the viral particle inhibits angiogenesis in the tumor of the vertebrate subject.

36. The method of claim 30, wherein the viral particle targets or images embryonic vasculature.

37. The method of claim 1, further comprising decreasing an immune response to the viral particles.

38. The method of claim 37, further comprising coating the viral particles with polyethylene glycol or glucose.

39. The method of claim 37, wherein the viral particle targets or images blood flow in the vertebrate subject.

40. The method of claim 30, wherein the viral particle targets or images atherosclerosis, ischemia, or stroke in the mammal.

41. The method of claim 23 wherein the plurality of targeting/imaging molecules are polypeptides.

42. The method of claim 7, wherein the polypeptides are viral antigens or bacterial antigens.

43. The method of claim 42, wherein the polypeptides are animal viral antigens or animal bacterial antigens.

44. The method of claim 41, wherein the polypeptides target or image the viral particle to VEGF-1 receptor or Flk-1/VEGF-2 receptor on tumor vascular endothelium.

45. The method of claim 41, wherein the peptides target or image atherosclerosis, ischemia, or stroke.

46. The method of claim 41, wherein the polypeptides are antibodies.

47. The method of claim 46, wherein the antibodies target or image the viral particle to tumor specific antigens on a tumor in a live mammal.

48. The method of claim 47 wherein the antibodies target or image the viral particle to VEGF-1 receptor, Flk-1/VEGF-2 receptor, LyP1 tumor target, lung endothelium, α5β1 integrin on colorectal carcinoma, nasopharyngeal carcinoma, αvβ3 integrin on breast, lung, brain, bone, liver, or kidney carcinomas.

49. The method of claim 1, further comprising encapsidating a therapeutic or diagnostic agent in the viral particle.

50. The method of claim 49, wherein the therapeutic agent is a nucleic acid, siRNA, shRNA, antisense RNA, dendrimer, aptamer, small molecule, polypeptide, or endotoxin.

51. The method of claim 50, wherein the therapeutic agent treats vascular disease, atherosclerosis, ischemia, stroke, cancer or infectious disease.

52. The method of claim 50, wherein the therapeutic agent is an anti-tumor agent, an anti-infective agent, an anti-angiogenesis agent, or an apoptosis inducer.

53. The method of claim 49, wherein the diagnostic agent is a cell marker, green fluorescent protein, or luciferase.

54. A method for treating or preventing a disease in a vertebrate subject comprising, administering to the vertebrate subject a plant viral particle comprising a plurality of targeting/imaging molecules directed to a tissue of the vertebrate subject, wherein the targeting/imaging molecule binds to the tissue to treat or prevent the disease of the vertebrate subject.

55. The method of claim 54 wherein the plurality of targeting/imaging molecules are ligands that binds to a cell surface receptor in the tissue of the vertebrate subject.

56. The method of claim 55 wherein the tissue is a vasculature in the vertebrate subject.

57. The method of claim 56 wherein the tissue is a tumor vasculature in the vertebrate subject.

58. The method of claim 55 wherein the tissue is a tumor in the vertebrate subject.

59. The method of claim 57 wherein the cell surface receptor is VEGF-1 receptor or Flk-1/VEGF-2 receptor.

60. The method of claim 58 wherein the cell surface receptor is VEGF-1 receptor, Flk-1/VEGF-2 receptor, LyP1 tumor target, lung endothelium, α5β1 integrin on colorectal carcinoma, nasopharyngeal carcinoma, αvβ3 integrin on breast, lung, brain, bone, liver, or kidney carcinomas.

61. The method of claim 55, wherein the plurality of targeting/imaging molecules are polyethylene glycol conjugated to ligands.

62. The method of claim 54 wherein the plurality of targeting/imaging molecules are attached by chemical crosslink to the viral particle.

63. The method of claim 62, wherein the plurality of targeting/imaging molecules are small molecules, metal complexes, polymer, carbohydrates, polypeptides, polynucleotides, or fluorescent chemical molecule.

64. The method of claim 63, wherein the plurality of targeting/imaging molecules are polyethylene glycol conjugated to the targeting/imaging molecule.

65. The method of claim 63, wherein the plurality of targeting/imaging molecules are transferrin, RGD-containing polypeptide, protective antigen of anthrax toxin, neuropeptide Y, glycopolymer, polyethylene glycol, or folic acid.

66. The method of claim 65, wherein the plurality of targeting/imaging molecules are polyethylene glycol conjugated to folic acid.

67. The method of claim 54 wherein the plurality of targeting/imaging molecules are Egf17 polypeptides or fragments thereof.

68. The method of claim 54 wherein the plurality of targeting/imaging molecules are exogenous polypeptides encoded by a viral particle genome.

69. The method of claim 68 wherein the plurality of targeting/imaging molecules are polypeptides binding a therapeutic or diagnostic agent.

70. The method of claim 69 wherein the plurality of targeting/imaging molecules are polypeptides binding doxorubicin, verapamil, vincristine, or vinblastine.

71. The method of claim 54 wherein the plurality of targeting/imaging molecules are antibodies that binds to the cell surface receptor in the vasculature.

72. The method of claim 54, wherein the plurality of targeting/imaging molecules are fluorescent dye, MRI contrast agent, PET contrast agent, or CT contrast agent.

73. The method of claim 54 further comprising administering the plant viral particle to the subject via an oral, pulmonary, oropharyngeal, or nasopharyngeal route.

74. The method of claim 54 further comprising administering the plant viral particle to the subject via parenteral, topical, intravenous, oral, subcutaneous, intraarterial, intracranial, intraperitoneal, intranasal or intramuscular route.

75. The method of claim 54 wherein the plurality of targeting/imaging molecules induce a cell mediated immune response to a tumor cell, virus, bacteria, or parasite.

76. The method of claim 75 wherein the plurality of targeting/imaging molecules are tumor antigens, viral antigens, bacterial antigens, or parasite antigens.

77. The method of claim 54 wherein the disease is cancer, solid tumor or infectious disease.

78. The method of claim 77, further comprising administering to the subject a therapeutic agent in the plant viral particle.

79. The method of claim 78, wherein the therapeutic agent is a polypeptide, a nucleic acid, siRNA, shRNA, antisense RNA, dendrimer, aptamer, antibody, endotoxin, or a small molecule.

80. The method of claim 79, wherein the therapeutic agent is an immune system modulator.

81. The method of claim 79, wherein the therapeutic agent is an anti-tumor agent, an anti-infective agent, an anti-angiogenesis agent, or an apoptosis inducer.

82. The method of claim 79, wherein the therapeutic agent is an enzyme, an interleukin, an interferon, a cytokine, a chemokine, TNF, taxol, an antibody, or combinations of any two or more thereof.

83. The method of claim 81, wherein the anti-tumor agent is doxorubicin, verapamil, vincristine, or vinblastine.

84. The method of claim 82, wherein the therapeutic agent is IL-2, IL-3, IL-4, IL-5, IL-6, IL-7, IL-9, IL-12, IL-13, IL-15, interferon-α, interferon-β, interferon-γ, IP-10, I-TAC, MIG, functional derivatives of any thereof, or combinations of any two or more thereof.

85. The method of claim 54 wherein the disease is a vascular disease.

86. The method of claim 85, further comprising administering to the subject a therapeutic agent in the plant viral particle

87. The method of claim 86, wherein the therapeutic agent is a polypeptide, a nucleic acid, siRNA, shRNA, antisense RNA, dendrimer, aptamer, antibody, endotoxin, or a small molecule.

88. The method of claim 85, wherein the vascular disease is ischemia, stroke or atherosclerosis.

89. A plant viral particle comprising:
a viral subunit comprising a plurality of covalent attachment sites,
a plurality of targeting/imaging molecules covalently attached to the viral subunit, and
a plurality of viral subunits assembled into the viral particle displaying the plurality of targeting/imaging molecules on the viral particle.

90. The plant viral particle of claim 89 wherein the plurality of targeting/imaging molecules are attached by chemical crosslink to the viral particle.

91. The plant viral particle of claim 90 further comprising a plurality of lysine residues on the viral subunit covalently attached to the plurality of targeting/imaging molecules.

92. The plant viral particle of claim 89 wherein the plurality of targeting/imaging molecules are small molecules, metal complexes, polymer, carbohydrates, polypeptides, polynucleotides or fluorescent chemical molecule.

93. The method of claim 92, wherein the plurality of targeting/imaging molecules are polyethylene glycol conjugated to the targeting/imaging molecule.

94. The method of claim 92, wherein the plurality of targeting/imaging molecules are transferrin, RGD-containing polypeptide, protective antigen of anthrax toxin, neuropeptide Y, glycopolymer, polyethylene glycol, or folic acid.

95. The method of claim 94, wherein the plurality of targeting/imaging molecules are polyethylene glycol conjugated to folic acid.

96. The plant viral particle of claim 89 wherein the plurality of targeting/imaging molecules are encoded by an exogenous nucleotide sequence in a viral particle genome.

97. The plant viral particle of claim 96 wherein the exogenous nucleotide sequence encodes siRNA, shRNA, or antisense RNA.

98. The plant viral particle of claim 96 wherein the exogenous nucleotide sequence encodes a foreign polypeptide expressed as part of a coat protein of the viral particle.

99. The plant viral particle of claim 89 wherein the exogenous nucleotide sequence encodes a foreign polypeptide expressed as part of a βE-αF loop, βB-βC loop, C′-C″ loop, or an N-terminus of the coat protein of the viral particle.

100. The plant viral particle of claim 98 wherein the foreign polypeptide is a tumor antigen, a viral antigen, a bacterial antigen, or a parasite antigen.

101. The plant viral particle of claim 96 wherein the plurality of targeting/imaging molecules are ligands binding to tumor cell surface receptors.

102. The plant viral particle of claim 101 wherein the plurality of targeting/imaging molecules are ligands binding to VEGF-1 receptor, Flk-1/VEGF-2 receptor, LyP1 tumor target, lung endothelium, α5β1 integrin, or αvβ3 integrin.

103. The method of claim 101, wherein the plurality of targeting/imaging molecules are polyethylene glycol conjugated to ligands.

104. The plant viral particle of claim 89 wherein the plurality of targeting/imaging molecules induce a cell mediated immune response to a tumor cell, virus, bacteria, or parasite.

105. The plant viral particle of claim 101 wherein the plurality of targeting/imaging molecules are tumor antigens, viral antigens, bacterial antigens, or parasite antigens.

106. The plant viral particle of claim 96 wherein the plurality of targeting/imaging molecules are peptides binding a therapeutic or diagnostic agent.

107. The plant viral particle of claim 106 wherein the plurality of targeting/imaging molecules are peptides binding doxorubicin, verapamil, vincristine, or vinblastine.

108. The plant viral particle of claim 89, wherein the vertebrate subject is a mammalian subject or an avian subject.

109. The plant viral particle of claim 89 wherein the targeting/imaging molecules target or image the viral particles in a vasculature in a vertebrate subject.

110. The plant viral particle of claim 109, wherein the targeting/imaging molecule is a fluorescent molecule for fluorescent imaging, gadolinium chelate molecule for magnetic resonance imaging, PET contrast agent or CT contrast agent.

111. The plant viral particle of claim 89 further comprising the plurality of targeting/imaging molecules displayed on the surface of the viral particle.

112. The plant viral particle of claim 89 further comprising the plurality of targeting/imaging molecules displayed on the interior of the viral particle.

113. The plant viral particle of claim 89, wherein the plant viral particle is a Comovirus, Tombusvirus, Sobemovirus, or Nepovirus.

114. The plant viral particle of claim 89 wherein the plant viral particle is a comovirus.

115. The plant viral particle of claim 89 wherein the comovirus is a cowpea mosaic virus.

116. The plant viral particle of claim 109 wherein the viral particle targets or images a vascular endothelium in the vertebrate subject.

117. The plant viral particle of claim 116, wherein the viral particle targets or images the vascular endothelium to distinguish veins from arteries.

118. The plant viral particle of claim 116, wherein the viral particle targets or images tumor vasculature.

119. The plant viral particle of claim 118, wherein the plurality of targeting/imaging molecules are ligands binding to a receptor on the tumor vasculature.

120. The plant viral particle of claim 119, wherein the plurality of targeting/imaging molecules are ligands binding to VEGF-1 receptor or Flk-1/VEGF-2 receptor.

121. The plant viral particle of claim 119, wherein the viral particle inhibits angiogenesis in the tumor of the vertebrate subject.

122. The plant viral particle of claim 116, wherein the viral particle images embryonic vasculature.

123. The plant viral particle of claim 109, further comprising having a decreased immune response to the viral particle in the vertebrate subject.

124. The plant viral particle of claim 123, further comprising polyethylene glycol or glucose coating the viral particle.

125. The plant viral particle of claim 123, wherein the viral particle targets or images blood flow in the vertebrate subject.

126. The plant viral particle of claim 116, wherein the viral particle targets or images atherosclerosis, ischemia, or stroke in the vertebrate subject.

127. The plant viral particle of claim 109 wherein the plurality of targeting/imaging molecules are polypeptides.

128. The plant viral particle of claim 92, wherein the polypeptides are viral antigens or bacterial antigens.

129. The plant viral particle of claim 128, wherein the polypeptides are animal viral antigens or animal bacterial antigens.

130. The plant viral particle of claim 127, wherein the polypeptides target or image the viral particle to a cell surface receptor in the vertebrate subject.

131. The plant viral particle of claim 118, wherein the peptides target or image the viral particle to VEGF-1 receptor or Flk-1/VEGF-2 receptor on the tumor vascular endothelium.

132. The plant viral particle of claim 127, wherein the peptides target or image atherosclerosis, ischemia, or stroke.

133. The plant viral particle of claim 127, wherein the peptides are antibodies.

134. The plant viral particle of claim 133, wherein the antibodies target or image the viral particle to tumor specific antigens on a tumor in a live mammal.

135. The plant viral particle of claim 134 wherein the antibodies target or image the viral particle to VEGF-1 receptor, Flk-1/VEGF-2 receptor, LyP1 tumor target, lung endothelium, α5β1 integrin on colorectal carcinoma, nasopharyngeal carcinoma, αvβ3 integrin on breast, lung, brain, bone, liver, or kidney carcinomas.

136. The plant viral particle of claim 109, further comprising a therapeutic or diagnostic agent encapsidated in the viral particle.

137. The plant viral particle of claim 136, wherein the therapeutic agent is a nucleic acid, siRNA, shRNA, antisense RNA, dendrimer, aptamer, small molecule, polypeptide, or endotoxin.

138. The plant viral particle of claim 137, wherein the therapeutic agent treats vascular disease, atherosclerosis, ischemia, or stroke.

139. The plant viral particle of claim 137, wherein the therapeutic agent is an anti-tumor agent, an anti-infective agent, an anti-angiogenesis agent, or an apoptosis inducer.

140. The plant viral particle of claim 136, wherein the diagnostic agent is a cell marker, green fluorescent protein, or luciferase.
